(19) United States
(12) Patent Application Publication
(10) Pub. No.: US 2026/0069803 A1
(43) Pub. Date: Mar. 12, 2026

(54) DETERMINING WORK OF BREATHING IN RESPIRATORY FLOW THERAPY SYSTEMS

(71) Applicant: Fisher & Paykel Healthcare Limited, Auckland (NZ)

(72) Inventor: Anton Kim GULLEY, Auckland (NZ)

(21) Appl. No.: 19/107,355

(22) PCT Filed: Oct. 27, 2023
(86) PCT No.: PCT/IB2023/060841

§ 371 Date: Feb. 27, 2025

Related U.S. Application Data
(60) Provisional application No. 63/381,465, filed on Oct. 28, 2022.

(51) Int. Cl. A61M 16/00; A61M 16/06; A61M 16/10

(52) U.S. Cl. CPC A61M 16/024; A61M 16/0051; A61M 16/06; A61M 16/1045; A61M 2016/0033; A61M 2205/18; A61M 2205/3334; A61M 2205/42; A61M 2205/502

(57) ABSTRACT

A respiratory apparatus configured to provide a flow of gases to a user for respiratory therapy. The apparatus comprises a flow generator configured to generate the flow of gases for the user and a sensor or sensors that are configured to generate flow parameter data indicative or representative of the flow of gases. A controller is configured to receive the flow parameter data, determine a nasal pressure variation value indicative of the user's average nasal pressure based at least partly on the received flow parameter data, determine a work of breathing (WOB) indicator based at least partly on the determined nasal pressure variation value, and initiate one or more actions based at least partly on the determined WOB indicator.

### FIELD OF THE DISCLOSURE

This disclosure relates to determining work of breathing during use of an unsealed respiratory apparatus (i.e. open respiratory apparatus) by a patient.

### BACKGROUND

Breathing assistance apparatuses are used in various environments such as hospital, medical facility, residential care, or home environments to deliver a flow of gases to users or patients. A breathing assistance or respiratory therapy apparatus (collectively, “respiratory apparatus” or “respiratory devices”) may be used to deliver supplementary oxygen or other gases with a flow of gases, and/or a humidification apparatus to deliver heated and humidified gases. A respiratory apparatus may allow adjustment and control over characteristics of the gases flow, including flow rate, temperature, gases concentration, humidity, pressure, etc. Sensors, such as flow sensors and/or pressure sensors are used to measure characteristics of the gases flow.

### SUMMARY

In a first aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and initiate one or more actions based at least partly on the determined WOB indicator.

In a second aspect, the present disclosure broadly comprises a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and initiate one or more actions based at least partly on the determined WOB indicator.

The respiratory apparatus of the first aspect or the respiratory therapy system of the second aspect may further have any one or more of the following aspects or features defined in the following paragraphs.

In a configuration, the flow parameter data comprises flow rate data indicative or representative of the flow rate of the flow of gases provided by the flow generator.

In a configuration, the apparatus comprises a flow rate sensor or sensors that are configured to sense and generate the flow rate data.

In a configuration, the flow rate sensor or sensors are positioned or located in or within a flow path of the flow of gases.

In a configuration, the flow rate sensor or sensors are positioned or located at or near an outlet of a blower of the flow generator.

In a configuration, the flow rate sensor or sensors are in electrical communication with the controller.

In a configuration, the controller is further configured to process the flow rate data to remove noise and/or signal components associated with the flow generator.

In a configuration, the controller is configured to remove noise relating to the effect of a motor on the flow rate data.

In a configuration, the controller is configured to receive data regarding a motor speed, and the flow rate data of the flow of gases is discarded if the motor speed is below a pre-set threshold.

In a configuration, the controller is configured to discard the flow rate data if the controller determines the flow rate data parameter of the flow of gases is of insufficient quality.

In a configuration, the flow rate data is determined to be of insufficient quality if it includes large transient peaks.

In a configuration, the flow parameter data comprises pressure data indicative or representative of the pressure of the flow of gases at an outlet of the blower of the flow generator.

In a configuration, the apparatus further comprises a pressure sensor or sensors that are configured to sense and generate the pressure data.

In a configuration, the pressure sensor or sensors are positioned or located in or within a flow path of the flow of gases.

In a configuration, the pressure sensor or sensors are positioned or located at or near an outlet of a blower of the flow generator.

In a configuration, the pressure sensor or sensors are in electrical communication with the controller.

In a configuration, the controller is further configured to determine an initial nasal pressure estimate value indicative or representative of an estimate of the user's nasal pressure based at least partly on the pressure data.

In a configuration, the controller is further configured to determine a flow path conductance estimate indicative or representative of an estimate of the conductance of the flow path for the flow of gases between the flow generator and a patient interface.

In a configuration, the controller is configured to determine the flow path conductance estimate at least partly based on an initial nasal pressure estimate value indicative or representative of an estimate of the user's nasal pressure.

In a configuration, the controller is configured to determine the flow path conductance estimate at least partly based on flow rate data indicative or representative of the flow rate of the flow of gases provided by the flow generator.

In a configuration, the controller is configured to determine the flow path conductance estimate at least partly based on pressure data indicative or representative of the pressure of the flow of gases at an outlet of the blower of the flow generator.

In a configuration, the controller is configured to determine the flow path conductance estimate at least partly based on flow rate data indicative of representative of the flow rate of the flow of gases provided by the flow generator and motor speed representing the motor speed of a blower of the flow generator.

In a configuration, the controller is configured to determine the flow path conductance estimate at least partly based on flow rate data indicative or representative of the flow rate of the flow of gases provided by the flow generator and pressure data indicative or representative of the pressure of the flow of gases at an outlet of the blower of the flow generator.

In a configuration, the controller is configured to determine the nasal pressure variation value at least partly based on the flow path conductance estimate.

In a configuration, the controller is configured to determine the nasal pressure variation value at least partly based on flow rate data indicative or representative of the flow rate of the flow of gases provided by the flow generator.

In a configuration, the controller is configured to determine the nasal pressure variation value at least partly based on minute ventilation data indicative or representative of the average volume of gases being provided by the flow generator per minute.

In a configuration, the controller is configured to determine minute ventilation data by fitting a plurality of splines to the flow parameter data of the flow of gases, wherein the plurality of splines is fit using the least squares criterion and the minute ventilation data is determined by integrating along the plurality of splines.

In a configuration, the controller is configured to determine minute ventilation data by determining the integral of the absolute value of the first term of a line fitted to the flow parameter data of the flow of gases.

In a configuration, the controller is configured to determine device minute ventilation data by determining the integral of the absolute value of a line fitted to the data of the flow parameter data of the flow of gases, divided by a time range.

In a configuration, the controller is configured to determine device minute ventilation data by determining an average of absolute values of a line fitted to the flow parameter data of the flow of gases across a range of time-points within a time range.

In a configuration, the nasal pressure variation value is determined at a frequency selected in the range of 1 Hz to 20 Hz.

In a configuration, the nasal pressure variation value is determined continuously as a rolling average value.

In a configuration, the apparatus further comprises a non-transitory computer-readable medium that is accessible or in data communication with the controller, and preferably wherein the non-transitory computer-readable medium comprises a non-volatile memory, and preferably wherein the apparatus further comprises a patient nostril model that is stored in the non-volatile memory.

In a configuration, the controller is further configured to determine a user breath flow rate estimate value indicative or representative of the user's breath flow rate at least partly based on flow rate data indicative or representative of the flow rate of the flow of gases provided by the flow generator and the patient nostril model.

In a configuration, the controller is configured to determine the user breath flow rate estimate value at least partly based on a flow path conductance estimate indicative or representative of an estimate of the conductance of the flow path for the flow of gases between the flow generator and a patient interface.

In a configuration, the controller is configured to determine the user breath flow rate estimate value at least partly based on minute ventilation data indicative or representative of the average volume of gases being provided by the flow generator per minute.

In a configuration, the controller is configured to determine a nares conductance estimate indicative or representative of an estimate of the conductance of the flow path for the flow of gases between a patient interface and the user's nostrils at least partly based on data indicative of the patient interface size and an estimate of nostril occlusion by the patient interface.

In a configuration, the controller is configured to determine the user breath flow rate estimate value at least partly based on the determined or calculated nares conductance estimate.

In a configuration, the controller is configured to determine the work of breathing indicator at least partly based on the nasal pressure variation value and the user breath flow rate estimate signal.

In a configuration, the controller is configured to determine a smoothness value indicative or representative of the smoothness of minute ventilation data indicative or representative of the average volume of gases being provided by the flow generator per minute.

In a configuration, the controller is configured to determine the work of breathing indicator at least partly based on the nasal pressure variation value and the smoothness value.

In a configuration, the apparatus further comprises a display screen, and preferably wherein the display screen displays a graphical user interface, and/or preferably wherein the display screen is in electrical communication with the controller.

In a configuration, the display screen is removable from the apparatus or a housing of the apparatus.

In a configuration, the controller is configured to display a graphical indicator representing the determined work of breathing indicator on the display screen.

In a configuration, the graphical indicator comprises any one or more of the following: numerical value, text, waveform, illustration, or animation.

In a configuration, the graphical indicator is indicative or representative of whether the determined work of breathing indicator is increasing or decreasing.

In a configuration, the controller is configured to trigger or generate an alert, alarm, and/or notification based at least partly on the determined work of breathing indicator and one or more thresholds.

In a configuration, the alert, alarm, and/or notification is triggered or generated based at least partly on determining that the work of breathing indicator has increased above a threshold.

In a configuration, the alert, alarm, and/or notification is triggered or generated based at least partly on determining that the work of breathing indicator has decreased below a threshold.

In a configuration, the threshold is a disconnection detection threshold.

In a configuration, the alert, alarm, and/or notification is triggered or generated based at least partly on determining that the work of breathing indicator has decreased below the threshold continuously for an associated predetermined duration condition.

In a configuration, the controller is configured to generate the alert, alarm, and/or notification in a form selected from any one or more of the following: audible, visual, and/or tactile.

In a configuration, the apparatus further comprises an audio output device in electrical communication with the controller, and wherein the controller is configured to generate the alert, alarm, and/or notification audibly via the audio output device.

In a configuration, the controller is configured to generate the alert, alarm, and/or notification visually via a display screen of the apparatus.

In a configuration, the controller is configured to send or transmit data representing the alert, alarm, and/or notification to a remote device or system that is in data communication with the apparatus.

In a configuration, the controller is operable to configure or adjust any parameters of or associated with the one or more of the thresholds based at least partly on user input via a graphical user interface of a display screen of the apparatus.

In a configuration, the controller is configured to generate or provide suggested thresholds and/or parameters associated with the one or more thresholds based at least partly on the work of breathing indicator.

In a configuration, the controller is further configured to determine a ratio or percentage representing the work of breathing indicator for the user relative to a nominal equivalent work of breathing indicator of a nominal average healthy person.

In a configuration, the nominal equivalent work of breathing indicator is determined based at least partly on an amplitude of nominal variations in nasal pressure of the nominal average healthy person.

In a configuration, the amplitude of nominal variations in nasal pressure of the nominal average healthy person is determined based at least partly on predetermined physiological parameters of the nominal average healthy person.

In a configuration, the amplitude of nominal variations in nasal pressure of the nominal average healthy person is determined based at least partly on manually-input physiological parameters related to the user.

In a configuration, the amplitude of nominal variations in nasal pressure of the nominal average healthy person is determined based at least partly on a nominal measure of nostril occlusion by nominal nasal cannula prongs of a patient interface.

In a configuration, the amplitude of nominal variations in nasal pressure of the nominal average healthy person is determined based at least partly on a manually-input measure of nostril occlusion by nasal cannula prongs of a patient interface.

In a configuration, the controller is configured to generate one or more alerts, alarms, and/or notifications based at least partly on the value of the ratio or percentage, representing the work of breathing indicator for the user relative to a nominal equivalent work of breathing indicator of a nominal average healthy person, or associated trend data of the ratio or percentage, and one or more thresholds.

In a configuration, the controller is configured to display the value of the ratio or percentage and/or trend data relating to the ratio or percentage visually on a display screen of the apparatus.

In a configuration, the controller is configured to generate an alert, alarm, and/or notification comprising data indicative of suggested adjustments to one or more therapy settings and/or apparatus settings based at least partly on the value of the ratio or percentage, representing the work of breathing indicator for the user relative to a nominal equivalent work of breathing indicator of a nominal average healthy person, or associated trend data of the ratio or percentage, and one or more thresholds.

In a configuration, the therapy settings and/or apparatus settings comprise a flow rate setting and/or a gas flow oxygen concentration setting (e.g. FiO₂ setting or FdO₂ setting).

In a configuration, the apparatus or system further comprises a housing, and wherein the housing comprises or integrates: the flow generator; a humidifier that is configured to heat and humidify the flow of gases; a sensing block or sensor module comprising the sensor or sensors that are configured to generate flow parameter data indicative or representative of the flow of gases; and the controller.

In a configuration, the sensing block or sensor module comprises a flow rate sensor and a pressure sensor.

In a third aspect, the present disclosure broadly comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data; determining a work of breathing (WOB) indicator based at least partly on the determined nasal pressure variation value; and initiating one or more actions based at least partly on the determined WOB indicator.

In a configuration, the flow parameter data comprises flow rate data indicative or representative of the flow rate of the flow of gases provided by the flow generator.

In a configuration, the apparatus comprises a flow rate sensor or sensors that are configured to sense and generate the flow rate data.

In a configuration, the flow rate sensor or sensors are positioned or located in or within a flow path of the flow of gases.

In a configuration, the flow rate sensor or sensors are positioned or located at or near an outlet of a blower of the flow generator.

In a configuration, the flow rate sensor or sensors are in electrical communication with the controller.

In a configuration, the method further comprises processing the flow rate data to remove noise and/or signal components associated with the flow generator.

In a configuration, the method comprises removing noise relating to the effect of a motor on the flow rate data.

In a configuration, the method comprises receiving data regarding a motor speed, and discarding the flow rate data of the flow of gases if the motor speed is below a pre-set threshold.

In a configuration, the method comprises discarding the flow rate data if it is determined that the flow rate data of the flow of gases is of insufficient quality.

In a configuration, the flow rate data is determined to be of insufficient quality if it includes large transient peaks.

In a configuration, the flow parameter data comprises pressure data indicative or representative of the pressure of the flow of gases at an outlet of the blower of the flow generator.

In a configuration, the apparatus further comprises a pressure sensor or sensors that are configured to sense and generate the pressure data.

In a configuration, the pressure sensor or sensors are positioned or located in or within a flow path of the flow of gases.

In a configuration, the pressure sensor or sensors are positioned or located at or near an outlet of a blower of the flow generator.

In a configuration, the pressure sensor or sensors are in electrical communication with the controller.

In a configuration, the method further comprises determining an initial nasal pressure estimate value indicative or representative of an estimate of the user's nasal pressure based at least partly on the pressure data.

In a configuration, the method further comprises determining a flow path conductance estimate indicative or representative of an estimate of the conductance of the flow path for the flow of gases between the flow generator and a patient interface.

In a configuration, the method comprises determining the flow path conductance estimate at least partly based on an initial nasal pressure estimate value indicative or representative of an estimate of the user's nasal pressure.

In a configuration, the method comprises determining the flow path conductance estimate at least partly based on flow rate data indicative or representative of the flow rate of the flow of gases provided by the flow generator.

In a configuration, the method comprises determining the flow path conductance estimate at least partly based on pressure data indicative or representative of the pressure of the flow of gases at an outlet of the blower of the flow generator.

In a configuration, the method comprises determining the flow path conductance estimate at least partly based on flow rate data indicative of representative of the flow rate of the flow of gases provided by the flow generator and motor speed representing the motor speed of a blower of the flow generator.

In a configuration, the method comprises determining the flow path conductance estimate at least partly based on flow rate data indicative or representative of the flow rate of the flow of gases provided by the flow generator and pressure data indicative or representative of the pressure of the flow of gases at an outlet of the blower of the flow generator.

In a configuration, the method comprises determining the nasal pressure variation value at least partly based on the flow path conductance estimate.

In a configuration, the method comprises determining the nasal pressure variation value at least partly based on flow rate data indicative or representative of the flow rate of the flow of gases provided by the flow generator.

In a configuration, the method comprises determining the nasal pressure variation value at least partly based on minute ventilation data indicative or representative of the average volume of gases being provided by the flow generator per minute.

In a configuration, the method comprises determining minute ventilation data by fitting a plurality of splines to the flow parameter data of the flow of gases, wherein the plurality of splines are fit using the least squares criterion and the minute ventilation data is determined by integrating along the plurality of splines.

In a configuration, the method comprises determining minute ventilation data by determining the integral of the absolute value of the first term of a line fitted to the flow parameter data of the flow of gases.

In a configuration, the method comprises determining device minute ventilation data by determining the integral of the absolute value of a line fitted to the data of the flow parameter data of the flow of gases, divided by a time range.

In a configuration, the method further comprises determining device minute ventilation data by determining an average of absolute values of a line fitted to the flow parameter data of the flow of gases across a range of time-points within a time range.

In a configuration, the method comprises determining the nasal pressure variation value at a frequency selected in the range of 1 Hz to 20 Hz.

In a configuration, the method comprises determining the nasal pressure variation value continuously as a rolling average value.

In a configuration, the apparatus further comprises a non-transitory computer-readable medium that is accessible or in data communication with the controller, and preferably wherein the non-transitory computer-readable medium comprises a non-volatile memory, and preferably wherein the apparatus further comprises a patient nostril model that is stored in the non-volatile memory.

In a configuration, the method further comprises determining a user breath flow rate estimate value indicative or representative of the user's breath flow rate at least partly based on flow rate data indicative or representative of the flow rate of the flow of gases provided by the flow generator and the patient nostril model.

In a configuration, the method comprises determining the user breath flow rate estimate value at least partly based on a flow path conductance estimate indicative or representative of an estimate of the conductance of the flow path for the flow of gases between the flow generator and a patient interface.

In a configuration, the method comprises determining the user breath flow rate estimate value at least partly based on minute ventilation data indicative or representative of the average volume of gases being provided by the flow generator per minute.

In a configuration, the method comprises determining a nares conductance estimate indicative or representative of an estimate of the conductance of the flow path for the flow of gases between a patient interface and the user's nostrils at least partly based on data indicative of the patient interface size and an estimate of nostril occlusion by the patient interface.

In a configuration, the method comprises determining the user breath flow rate estimate value at least partly based on the determined or calculated nares conductance estimate.

In a configuration, the method comprises determining the work of breathing indicator at least partly based on the nasal pressure variation value and the user breath flow rate estimate signal.

In a configuration, the method comprises determining a smoothness value indicative or representative of the smoothness of minute ventilation data indicative or representative of the average volume of gases being provided by the flow generator per minute.

In a configuration, the method comprises determining the work of breathing indicator at least partly based on the nasal pressure variation value and the smoothness value.

In a configuration, the apparatus further comprises a display screen, and preferably wherein the display screen displays a graphical user interface, and/or preferably wherein the display screen is in electrical communication with the controller.

In a configuration, the display screen is removable from the apparatus or a housing of the apparatus.

In a configuration, the method comprises displaying a graphical indicator representing the determined work of breathing indicator on the display screen.

In a configuration, the graphical indicator comprises any one or more of the following: numerical value, text, waveform, illustration, or animation.

In a configuration, the graphical indicator is indicative or representative of whether the determined work of breathing indicator is increasing or decreasing.

In a configuration, the method comprises triggering or generating an alert, alarm, and/or notification based at least partly on the determined work of breathing indicator and one or more thresholds.

In a configuration, the method comprises triggering or generating the alert, alarm, and/or notification based at least partly on determining that the work of breathing indicator has increased above a threshold.

In a configuration, the method comprises triggering or generating the alert, alarm, and/or notification based at least partly on determining that the work of breathing indicator has decreased below a threshold.

In a configuration, the threshold is a disconnection detection threshold.

In a configuration, the method comprises triggering or generating the alert, alarm, and/or notification based at least partly on determining that the work of breathing indicator has decreased below the threshold continuously for an associated predetermined duration condition.

In a configuration, the method comprises generating the alert, alarm, and/or notification in a form selected from any one or more of the following: audible, visual, and/or tactile.

In a configuration, the apparatus further comprises an audio output device in electrical communication with the controller, and wherein the method comprises to generating the alert, alarm, and/or notification audibly via the audio output device.

In a configuration, the method comprises generating the alert, alarm, and/or notification visually via a display screen of the apparatus.

In a configuration, the method comprises sending or transmitting data representing the alert, alarm, and/or notification to a remote device or system that is in data communication with the apparatus.

In a configuration, the method comprises configuring or adjusting any parameters of or associated with the one or more of the thresholds based at least partly on user input via a graphical user interface of a display screen of the apparatus.

In a configuration, the method comprises generating or providing suggested thresholds and/or parameters associated with the one or more thresholds based at least partly on the work of breathing indicator.

In a configuration, the method further comprises determining a ratio or percentage representing the work of breathing indicator for the user relative to a nominal equivalent work of breathing indicator of a nominal average healthy person.

In a configuration, the method comprises determining the nominal equivalent work of breathing indicator based at least partly on an amplitude of nominal variations in nasal pressure of the nominal average healthy person.

In a configuration, the method comprises determining the amplitude of nominal variations in nasal pressure of the nominal average healthy person based at least partly on predetermined physiological parameters of the nominal average healthy person.

In a configuration, the method comprises determining the amplitude of nominal variations in nasal pressure of the nominal average healthy person based at least partly on manually-input physiological parameters related to the user.

In a configuration, the method comprises determining the amplitude of nominal variations in nasal pressure of the nominal average healthy person based at least partly on a nominal measure of nostril occlusion by nominal nasal cannula prongs of a patient interface.

In a configuration, the method comprises determining the amplitude of nominal variations in nasal pressure of the nominal average healthy person based at least partly on a manually-input measure of nostril occlusion by nasal cannula prongs of a patient interface.

In a configuration, the method comprises generating one or more alerts, alarms, and/or notifications based at least partly on the value of the ratio or percentage, representing the work of breathing indicator for the user relative to a nominal equivalent work of breathing indicator of a nominal average healthy person, or associated trend data of the ratio or percentage, and one or more thresholds.

In a configuration, the method comprises displaying the value of the ratio or percentage and/or trend data relating to the ratio or percentage visually on a display screen of the apparatus.

In a configuration, the method comprises generating an alert, alarm, and/or notification comprising data indicative of suggested adjustments to one or more therapy settings and/or apparatus settings based at least partly on the value of the ratio or percentage, representing the work of breathing indicator for the user relative to a nominal equivalent work of breathing indicator of a nominal average healthy person, or associated trend data of the ratio or percentage, and one or more thresholds.

In a configuration, the therapy settings and/or apparatus settings comprise a flow rate setting and/or a gas flow oxygen concentration setting (e.g. FiO₂ setting or FdO₂ setting).

In a configuration, the apparatus further comprises a housing, and wherein the housing comprises or integrates: the flow generator; a humidifier that is configured to heat and humidify the flow of gases; a sensing block or sensor module comprising the sensor or sensors that are configured to generate flow parameter data indicative or representative of the flow of gases; and the controller.

In a configuration, the sensing block or sensor module comprises a flow rate sensor and a pressure sensor.

In fourth aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a user breath flow rate estimate value indicative or representative of the user's breath flow rate; determine a work of breathing (WOB) indicator based at least partly on the determined nasal pressure variation value and user breath flow rate estimate value; and initiating one or more actions based at least partly on the determined WOB indicator.

In a fifth aspect, the present disclosure broadly comprises a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a user breath flow rate estimate value indicative or representative of the user's breath flow rate; determine a work of breathing (WOB) indicator based at least partly on the determined nasal pressure variation value and user breath flow rate estimate value; and initiate one or more actions based at least partly on the determined WOB indicator.

In a sixth aspect, the present disclosure broadly comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data; determining a user breath flow rate estimate value indicative or representative of the user's breath flow rate; determining a work of breathing (WOB) indicator based at least partly on the determined nasal pressure variation value and user breath flow rate estimate value; and initiating one or more actions based at least partly on the determined WOB indicator.

In seventh aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a breath smoothness value indicative or representative of the rate of change of the user's nasal pressure fluctuations; determine a work of breathing (WOB) indicator based at least partly on the determined nasal pressure variation value and breath smoothness value; and initiating one or more actions based at least partly on the determined WOB indicator.

In an eighth aspect, the present disclosure broadly comprises a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a breath smoothness value indicative or representative of the rate of change of the user's nasal pressure fluctuations; determine a work of breathing (WOB) indicator based at least partly on the determined nasal pressure variation value and breath smoothness value; and initiate one or more actions based at least partly on the determined WOB indicator.

In a nineth aspect, the present disclosure broadly comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data; determine a breath smoothness value indicative or representative of the rate of change of the user's nasal pressure fluctuations; determining a work of breathing (WOB) indicator based at least partly on the determined nasal pressure variation value and breath smoothness value; and initiating one or more actions based at least partly on the determined WOB indicator.

The fourth-nineth aspects of the disclosure may further have any one or more of the features described in respect of the first-third aspects of the paragraphs above.

In a tenth aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; a display connected to or in data communication with the respiratory apparatus; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and display or cause display of WOB data on the display that is at least partly based on the determined WOB indicator.

In an eleventh aspect, the present disclosure comprises: a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a display connected to or in data communication with the respiratory system; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and display or cause display of WOB data on the display that is at least partly based on the determined WOB indicator.

The respiratory apparatus of the tenth aspect or the respiratory therapy system of the eleventh aspect may further have any one or more of the following aspects or features defined in the following paragraphs.

In a configuration, the display comprises a display screen of the respiratory apparatus.

In a configuration, the display screen is removable from the respiratory apparatus or a housing of the respiratory apparatus.

In a configuration, the display comprises a user interface of the respiratory apparatus.

In a configuration, the display comprises a graphical user interface (GUI).

In a configuration, the display is provided on a remote device or system that is in data communication with the respiratory apparatus.

In a configuration, the controller is further configured to transmit the WOB data for display to a remote device or system for display.

In a configuration, the controller is configured to display one or more graphical indicator(s) representing the WOB data on the display screen of the respiratory apparatus.

In a configuration, the graphical indicator(s) comprises any one or more of the following:

numerical value, text, waveform, illustration, or animation.

In a configuration, the graphical indicator may be indicative or representative of whether the determined WOB indicator is increasing or decreasing.

In a configuration, the WOB data displayed on the display comprises data indicative of a raw or absolute WOB indicator.

In a configuration, the controller is further configured to process the determined WOB indicator to generate a ratio or percentage representing the determined WOB indicator for the user relative to a nominal equivalent WOB indicator of a nominal average healthy person.

In a configuration, the WOB data displayed on the display comprises data indicative of a ratio or percentage representing the determined WOB indicator for the user relative to a nominal equivalent WOB indicator of a nominal average healthy person.

In a configuration, the controller is further configured to process a portion or window of the determined WOB indicator over time to generate one or more WOB indicator trends or trend data.

In a configuration, the WOB data displayed on the display comprises data indicative of one or more WOB indicator trends or trend data.

In a configuration, the WOB data displayed on the display comprises WOB indicator trends or trend data indicative of or representing any one or more of the following: WOB increasing, WOB decreasing, and/or WOB stable.

In a configuration, the WOB data displayed on the display may comprise data indicative of any one or more of the following data types: raw or absolute WOB indicator data, a ratio or percentage representing the determined WOB indicator for the user relative to a nominal equivalent WOB indicator of a nominal average healthy person, and/or WOB indicator trends or trend data.

In a configuration, one or more of the data types may be displayed in isolation or in combination with any one or more of the other data types on the display.

In a configuration, the data is displayed on a graphical user interface (GUI) of a display screen, and the GUI comprises a first GUI element configured to display WOB data of a first type, and a second GUI element configured to display WOB data of a second type.

In one example configuration, the first GUI element comprises a graphical indicator representing: raw or absolute WOB indicator data; and/or a ratio or percentage representing the determined WOB indicator for the user relative to a nominal equivalent WOB indicator of a nominal average healthy person, and the second GUI element comprises a graphical indicator representing: WOB indicator trends or trend data.

In a twelfth aspect, the present disclosure comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; a display connected to or in data communication with the respiratory apparatus; and a controller, wherein the method is executed or implemented by the controller and comprises the steps of: receiving the flow parameter data; determining a nasal pressure variation value indicative of the user's average nasal pressure based at least partly on the received flow parameter data; determining a work of breathing (WOB) indicator based at least partly on the determined nasal pressure variation value; and displaying or causing display of WOB data on the display that is at least partly based on the determined WOB indicator.

The method of the twelfth aspect may comprise any one or more of the features mentioned in respect of the tenth or eleventh aspects of the disclosure as described in the above paragraphs.

In a thirteenth aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more alerts, alarms, and/or notifications based at least partly on comparing the determined WOB indicator or associated WOB data to one or more thresholds.

In a fourteenth aspect, the present disclosure comprises: a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more alerts, alarms, and/or notifications based at least partly on comparing the determined WOB indicator or associated WOB data to one or more thresholds.

The respiratory apparatus of the thirteenth aspect or the respiratory therapy system of the fourteenth aspect may further have any one or more of the following aspects or features defined in the following paragraphs.

In a configuration, the controller is configured to generate the alert, alarm, and/or notification based at least partly on determining that the WOB indicator or associated WOB data has increased above a threshold.

In a configuration, the controller is configured to generate the alert, alarm, and/or notification based at least partly on determining that the WOB indicator or associated WOB data has decreased below a threshold.

In a configuration, the controller is configured to generate the alert, alarm, and/or notification in a form selected from any one or more of the following: audible, visual, and/or tactile.

In a configuration, the apparatus or system further comprises an audio output device in electrical communication with the controller, and wherein the controller is configured to generate the alert, alarm, and/or notification audibly via the audio output device.

In a configuration, the apparatus or system further comprises a display in electrical or data communication with the controller, and the controller is configured to generate the alert, alarm, and/or notification visually via the display.

In a configuration, the controller is configured to send or transmit data representing the generated alert, alarm, and/or notification to a remote device or system that is in data communication with the apparatus or system.

In a configuration, the controller is configured to send or transmit the determined WOB indicator and/or associated WOB data to a remote device or system that is in data communication with the apparatus or system.

In a configuration, the remote device or system displays or presents the data representing the alert, alarm and/or notification (e.g., whether visual, audible and/or tactile), and/or relays/transmits the alert, alarm and/or notification to another remote electronic device or system.

In a configuration, the display comprises a display screen of the respiratory apparatus.

In a configuration, the display screen is removable from the respiratory apparatus or a housing of the respiratory apparatus.

In a configuration, the display comprises a user interface of the respiratory apparatus.

In a configuration, the display comprises a graphical user interface (GUI).

In a configuration, the display is provided on a remote device or system that is in data communication with the respiratory apparatus.

In a configuration, the controller is configured to display a graphical indicator(s) representing the generated alert, alarm, and/or notification on the display screen of the respiratory apparatus.

In a configuration, the graphical indicator(s) comprises any one or more of the following: numerical value, textual information, graphical form or formats, trend lines, plotted or graphed data over time, waveform, illustration, icons, animation, and/or colour-coded information.

In a configuration, the controller is configured to display concurrently data indicative of or representing the determined WOB indicator or associated WOB data, and data indicative of the generated alert, alarm, and/or notification.

In a configuration, the controller is configured to generate one or more different types of alerts, alarms and/or notifications based at least partly on comparing the determined WOB indicator or associated WOB data to one or more thresholds or threshold criteria.

In a configuration, the controller is configured to determine a WOB status of the user based at least partly on comparing the determined WOB indicator or associated WOB data to one or more thresholds or threshold criteria.

In a configuration, the controller is configured to generate a first type of alert, alarm and/or notification that comprises data indicative of the determined WOB status (e.g. current status and/or trend status) of the user. In one example configuration, the determined WOB status may be selected from any one or more of the following: WOB increasing, WOB decreasing, WOB stable, high WOB, and/or low WOB.

In a configuration, the controller is configured to generate a second type of alert, alarm and/or notification that comprises data indicative of suggested actions to remediate or in response to the determined WOB status of the user. In one example configuration, the suggested actions may be selected from any one or more of the following: check patient, adjust therapy settings, and/or suggested changes to therapy settings (e.g., increase or decrease flow rate setting).

In a configuration, the controller may be configured such that the second type of alert, alarm and/or notification may be triggered or generated in response to the generation or triggering of the first type of alert, alarm and/or notification.

In a configuration, the controller may be configured to display data indicative of the first type of alert, alarm and/or notification concurrently with data indicative of the second type of alert, alarm and/or notification.

In a configuration, the determined WOB indicator or associated WOB data displayed comprises data indicative of any one or more of: a raw or absolute WOB indicator, a ratio or percentage representing the determined WOB indicator for the user relative to a nominal equivalent WOB indicator of a nominal average healthy person, and/or one or more WOB indicator trends or trend data.

In a configuration, the data is displayed on a graphical user interface (GUI) of a display screen, and the GUI comprises one or more GUI elements or panes or regions for displaying one or more of the generated alerts, alarms, and/or notifications.

In a configuration, the data displayed on the GUI of the display screen comprises a first GUI element or pane or region for displaying a first type of alert, alarm, and/or notification indicative of the determined WOB status of the user, and a second GUI element or pane or region for displaying a second type of alert, alarm, and/or notification indicative of suggested actions to remediate or in response to the determined WOB status of the user.

In a configuration, the controller may be configured to generate the first and/or second types of alerts, alarms, and/or notifications audibly and/or with one or more audible cues or voice commands over an associated audio output device.

In a fifteenth aspect, the present disclosure comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data; determining a work of breathing (WOB) indicator based at least partly on the determined nasal pressure variation value; and generating one or more alerts, alarms, and/or notifications based at least partly on comparing the determined WOB indicator or associated WOB data to one or more thresholds.

The method of the fifteenth aspect may comprise any one or more of the features mentioned in respect of the thirteenth or fourteenth aspects of the disclosure as described in the above paragraphs.

In a sixteenth aspect, the present disclosure broadly comprises a system comprising: a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and send or transmit the WOB indicator or associated WOB data to a remote device or system in data communication with the respiratory apparatus, and wherein: the remote device or system is configured to generate one or more alerts, alarms, and/or notifications based at least partly on comparing the determined WOB indicator or associated WOB data to one or more thresholds.

In a configuration, the remote device or system is configured to present the generated one or more alerts, alarms and/or notifications (e.g., whether visual, audible, and/or tactile).

In a configuration, the remote device or system is configured to push or transmit or relay the WOB indicator and/or associated WOB data and/or the generated one or more alerts, alarms, and/or notifications to another electronic device or system.

The system of the sixteenth aspect may further comprise any one or more of the features mentioned in respect of the thirteenth-fifteenth aspects of the disclosure in the paragraphs above. In one example, the remote device or system may be configured to carry out any one or more of the functions of the controller of the respiratory apparatus or system, including the generation and/or presentation (e.g. display or audible presentation) of the alarms, alerts, and/or notifications.

In a seventeenth aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more therapy parameter setting adjustment suggestions based at least partly on the determined WOB indicator or associated WOB data.

In an eighteenth aspect, the present disclosure comprises: a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more therapy parameter setting adjustment suggestions based at least partly on the determined WOB indicator or associated WOB data.

The respiratory apparatus of the seventeenth aspect or the respiratory therapy system of the eighteenth aspect may further have any one or more of the following aspects or features defined in the following paragraphs.

In a configuration, the controller is configured to generate the one or more therapy parameter setting adjustment suggestions based at least partly on comparing the determined WOB indicator or associated WOB data to one or more thresholds.

In a configuration, the WOB indicator or associated WOB data may comprise data indicative or representative of any one or more of: a raw or absolute WOB indicator, a ratio or percentage representing the determined WOB indicator for the user relative to a nominal equivalent WOB indicator of a nominal average healthy person, and/or one or more WOB indicator trends or trend data.

In a configuration, the controller may be configured to display or present the generated therapy parameter setting adjustment suggestions on a display screen of the respiratory apparatus.

In a configuration, the controller may be configured to transmit, send or relay the generated therapy parameter setting adjustment suggestions to one or more remote devices or systems in data communication with the respiratory apparatus or system.

In a configuration, the controller may be configured to apply the generated therapy parameter setting adjustment suggestions to the therapy settings (e.g. flow rate setting and/or gas flow oxygen concentration settings such as FiO₂ settings and/or FdO₂ settings). settings) of respiratory apparatus in response to input or confirmation from a user or clinician via a user interface of the respiratory apparatus and/or remote device or system.

In a nineteenth aspect, the present disclosure comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data;

determining a work of breathing (WOB) indicator based at least partly on the determined nasal pressure variation value; and generating one or more therapy parameter setting adjustment suggestions based at least partly on the determined WOB indicator or associated WOB data.

The method of the nineteenth aspect may comprise any one or more of the features mentioned in respect of the seventeenth or eighteenth aspects of the disclosure as described in the above paragraphs.

In a twentieth aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and process the WOB indicator or associated WOB data to detect a disconnection event.

In a twenty-first aspect, the present disclosure comprises: a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and process the WOB indicator or associated WOB data to detect a disconnection event.

The respiratory apparatus of the twentieth aspect or the respiratory therapy system of the twenty-first aspect may further have any one or more of the following aspects or features defined in the following paragraphs.

In a configuration, the disconnection event may be indicative or representative of a disconnection of any part of a flow path (e.g. patient breathing circuit and/or patient interface), and/or disconnection or detachment of a user from the patient interface.

In a configuration, the controller may be configured to detect a disconnection event at least partly based on whether the determined WOB indicator or associated WOB data reaches zero or crosses below a pre-determined threshold (e.g. near zero) for a predetermined time period.

In a configuration, the controller may be further configured to generate an alert, alarm and/or notification in response to detecting a disconnection event.

In a configuration, the controller may be configured to display or present the generated alert, alarm and/or notification of the disconnection event on a display of the respiratory apparatus.

In a configuration, the controller may be configured to send, transmit, or relay the generated alert, alarm and/or notification of the disconnection event to a remote system.

In a configuration, the generated alert, alarm and/or notification of the disconnection event may further comprise data indicative of suggested remedial or corrective action to resolve the disconnection event.

In a twenty-second aspect, the present disclosure comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data; determining a work of breathing (WOB) indicator based at least partly on the determined nasal pressure variation value; and processing the WOB indicator or associated WOB data to detect a disconnection event.

The method of the twenty-second aspect may comprise any one or more of the features mentioned in respect of the twentieth or twenty-first aspects of the disclosure as described in the above paragraphs.

In a twenty-third aspect, the present disclosure broadly comprises a respiratory apparatus configured to provide a flow of gases to a user for respiratory therapy, comprising: a flow generator configured to generate the flow of gases for the user; a sensor or sensors that are configured to generate flow parameter data indicative or representative of the flow of gases; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more alerts, alarms, and/or notifications based at least partly on comparing the determined WOB indicator or associated WOB data to one or more configurable thresholds.

In a twenty-fourth aspect, the present disclosure comprises: a respiratory therapy system configured to provide a flow of gases to a user for respiratory therapy, comprising: a flow generator configured to generate the flow of gases for the user; a sensor or sensors that are configured to generate flow parameter data indicative or representative of the flow of gases; a breathing conduit operatively coupled to the flow generator and configured to convey the flow of gases from the flow generator to the user; a patient interface operatively coupled to the breathing conduit; and a controller, wherein the controller is configured to: receive the flow parameter data; determine a nasal pressure variation value indicative of the user's average nasal pressure based at least partly on the received flow parameter data; determine a work of breathing (WOB) indicator based at least partly on the determined nasal pressure variation value; and generate one or more alerts, alarms, and/or notifications based at least partly on comparing the determined WOB indicator or associated WOB data to one or more configurable thresholds.

The respiratory apparatus of the twenty-third aspect or the respiratory therapy system of the twenty-fourth aspect may further have any one or more of the following aspects or features defined in the following paragraphs.

In a configuration, the controller is further operable to configure or adjust any parameters of or associated with the one or more of the thresholds based at least partly on user input via a graphical user interface (GUI) of a display screen of the apparatus.

In a configuration, the controller is configured to generate or provide suggested thresholds and/or parameters associated with the one or more thresholds based at least partly on the determined WOB indicator or associated WOB data.

In a configuration, the one or more configurable thresholds may comprise any one or more of: single thresholds, threshold ranges, upper and lower threshold limits, and/or threshold functions based on one or more parameters and/or conditions.

In a configuration, the apparatus or system provides a GUI on a display screen that is operable to adjust or configure the one or more thresholds associated with an alert, alarm and/or notification.

In a configuration, the GUI provides a first GUI element that presents data indicative of the alert, alarm, notification, and/or threshold or parameter and/or conditions of the threshold that is being adjusted, and a second GUI element that is user interactable or operable to adjust the one or more thresholds.

In a configuration, the second GUI element may comprise one or more user interactable GUI elements for adjusting the one or more configurable thresholds via touch input or interactivity with the display screen.

In a configuration, the one or more user interactable GUI elements for adjusting the one or more configurable thresholds may comprise any one or more of: toggle elements, dials, slider scale elements, selectable discrete threshold elements, and/or numerical and/or categorical input fields for inputting desired thresholds.

In a configuration, the one or more WOB alerts, alarms, and/or notifications may be configured with thresholds such that the comparison of the determined WOB indicator or

WOB data to the thresholds effectively functions as a surrogate alert, alarm and/or notification for other respiratory parameters.

In a configuration, the WOB alerts, alarms and/or notifications may be configured as surrogate alerts, alarms, and/or notifications for respiratory parameters selected from one or more of the following: respiratory rate, minute ventilation, and/or tidal volume.

In a configuration, the controller may be configured to compare the determined WOB indicator or associated WOB data to a plurality of configurable thresholds, and may generate one or more different alerts, alarms, or notifications depending on the outcome of each respective comparison.

In a configuration, the configurable thresholds may be preconfigured with default or suggested values.

In a configuration, a user interface may be provided on the display of the apparatus or system for accepting or adjusting the default or suggested thresholds for one or more of the alerts, alarms and/or notifications.

In a configuration, a user interface may be provided on a remote device or system that is in data communication with the respiratory apparatus or system, and the user interface may be operable by a user to accept or adjust the default or suggested thresholds for one or more of the alerts, alarms and/or notifications.

In a configuration, the alerts, alarms and/or notifications may be provided with a plurality of or multiple upper and lower bounds or thresholds.

In a configuration, the alerts, alarms and/or notifications may be provided with cascading or nested thresholds or threshold ranges, or inner and outer threshold ranges, or a plurality or series or progressive or escalating thresholds on a threshold scale. In such a configuration, the nature of the generated alert, alarm and/or notification associated with each respective threshold may be a function of or dependent on the nature, position, priority or extremity of the threshold on the overall threshold scale.

In a configuration, a high-priority alert, alarm or notification may be generated if a high-priority threshold is satisfied, and a low-priority alert, alarm and/or notification may be generated if a low-priority threshold is satisfied.

In a configuration, the default or suggested thresholds associated with any one or more of the alerts, alarms and/or thresholds may be recalibrated and/or dynamically altered by the controller based at least partly on changes in the determined WOB indicator or associated WOB data (e.g. trend data) over a therapy session, multiple therapy sessions, and/or some other configurable time period.

In a configuration, the one or more generated alerts, alarms and/or notifications may be configured to selectively present on one or more devices or systems based at least partly on the nature of the alert, alarm or notification and/or the threshold satisfied.

In a configuration, higher-priority alerts, alarms, and/or notifications may be configured to be presented on the respiratory apparatus and one or more other remote devices or systems.

In a configuration, lower-priority alerts, alarms, and/or notifications may be configured to be presented only on the respiratory apparatus.

In a twenty-fifth aspect, the present disclosure comprises a method of controlling a respiratory apparatus configured to provide a flow of gases to a user for respiratory therapy, the apparatus comprising: a flow generator configured to generate the flow of gases for the user; a sensor or sensors that are configured to generate flow parameter data indicative or representative of the flow of gases; and a controller, wherein the method is executed or implemented by the controller and comprises the steps of: receiving the flow parameter data; determining a nasal pressure variation value indicative of the user's average nasal pressure based at least partly on the received flow parameter data;

determining a work of breathing (WOB) indicator based at least partly on the determined nasal pressure variation value; and generating one or more alerts, alarms, and/or notifications based at least partly on comparing the determined WOB indicator or associated WOB data to one or more configurable thresholds.

The method of the twenty-fifth aspect may comprise any one or more of the features mentioned in respect of the twenty-third or twenty-fourth aspects of the disclosure as described in the above paragraphs.

In another aspect, the present disclosure comprises a respiratory apparatus comprising: a flow generator configured to generate a flow of gases for a user; a sensor or sensors that are configured to generate flow parameter data indicative or representative of a characteristic or parameter of the flow of gases; and a controller that is configured to: control the flow generator to deliver the flow of gases for nasal high flow therapy; determine a work of breathing (WOB) indicator based at least partly on the flow parameter data; and initiate one or more actions based at least partly on the determined WOB indicator.

In a configuration, the flow parameter data comprises pressure data indicative or representative of the pressure of the flow of gases.

In a configuration, the pressure data is sensed and generated by one or more pressure sensors that are in data communication with the controller.

In a configuration, the one or more pressure sensors are configured to sense and generate pressure data indicative or representative of the pressure of the flow of gases at an outlet of a blower of the flow generator.

In a configuration, the flow parameter data comprises flow rate data indicative or representative of the flow rate of the flow of gases.

In a configuration, the flow rate data is sensed and generated by one or more flow rate sensors that are in data communication with the controller.

In a configuration, the one or more flow rate sensors are position or located at or near an outlet of a blower of the flow generator.

In a configuration, the WOB indicator is determined at least partly based on flow parameter data that comprises sensed pressure and/or flow rate data relating to the flow of gases.

In another aspect, the present disclosure comprises a respiratory apparatus comprising: a flow generator configured to generate a flow of gases for a user; and a controller that is configured to: control the flow generator to deliver the flow of gases for respiratory therapy or nasal high flow therapy; determine a work of breathing (WOB) indicator based at least partly on pressure and/or flow rate measurements relating to the flow of gases; and initiate one or more actions based at least partly on the determined WOB indicator.

In another aspect, the present disclosure comprises a respiratory apparatus comprising: a flow generator configured to generate a flow of gases for a user; and a controller that is configured to: control the flow generator to deliver the flow of gases for respiratory therapy or nasal high flow therapy; and determine a work of breathing (WOB) indicator based at least partly on pressure and/or flow rate measurements relating to the flow of gases.

In another aspect, the present disclosure relates to an electronically implemented method comprising software code or coded instructions that are executable or implemented by a computer, processor, or controller to carry out any one or more of the methods or aspects described above.

In another aspect, the present disclosure broadly comprises a non-transitory computer-readable medium having stored thereon computer executable instructions that, when executed on a processing device or devices, cause the processing device or devices to perform or execute any one or more of the methods or aspects described above.

Any one of the aspects of the present disclosure described in the paragraphs above may further have any one or more of the features described in respect of any one or more of the other aspects of the present disclosure described in the paragraphs above.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows schematically a respiratory system configured to provide a respiratory therapy to a patient.

FIG. 2 is a front view of an example respiratory device with a humidification chamber in position and a raised handle/lever.

FIG. 3 is a top view corresponding to FIG. 2.

FIG. 4 is a right side view corresponding to FIG. 2.

FIG. 5 is a left side view corresponding to FIG. 2.

FIG. 6 is a rear view corresponding to FIG. 2.

FIG. 7 is a front left perspective view corresponding to FIG. 2.

FIG. 8 is a front right perspective view corresponding to FIG. 2.

FIG. 9 is a bottom view corresponding to FIG. 2.

FIG. 10 shows an example configuration of an air and oxygen inlet arrangement of a respiratory device.

FIG. 11 shows another example configuration of an air and oxygen inlet arrangement of the respiratory device.

FIG. 12 is a transverse sectional view showing further detail of the air and oxygen inlet arrangement of FIG. 11.

FIG. 13 is another transverse sectional view showing further detail of the air and oxygen inlet arrangement of FIG. 11.

FIG. 14 is a longitudinal sectional view showing further detail of the air and oxygen inlet arrangement of FIG. 11.

FIG. 15 is an exploded view of upper and lower chassis components of a main housing of the respiratory device.

FIG. 16 is a front left side perspective view of the lower chassis of the main housing showing a housing for receipt of a motor/sensor module sub-assembly.

FIG. 17 is a first underside perspective view of the main housing of the respiratory device showing a recess inside the housing for the motor/sensor module sub-assembly.

FIG. 18 is a second underside perspective view of the main housing of the respiratory device showing the recess for the motor/sensor module sub-assembly.

FIG. 19A illustrates a block diagram of a control system interacting with and/or providing control and direction to components of a respiratory system.

FIG. 19B illustrates a block diagram of an example controller.

FIG. 20 illustrates a block diagram of a motor and sensor module.

FIG. 21 illustrates a sensing chamber of an example motor and sensor module.

FIG. 22A illustrates an embodiment of a flow chart for a method of estimating device minute ventilation.

FIG. 22B illustrates another embodiment of a flow chart for a method of estimating device minute ventilation.

FIG. 22C illustrates another embodiment of a flow chart for a method of estimating device minute ventilation.

FIG. 22D illustrates another embodiment of a flow chart for a method of estimating device minute ventilation.

FIG. 23 illustrates another embodiment of a flow chart for a method of estimating device minute ventilation.

FIG. 24 illustrates a schematic example of a graphical user interface (GUI) display screen of the respiratory apparatus, showing an example configuration of a work of breathing monitor screen.

FIGS. 25A-25F illustrate schematic examples of a GUI display screen of the respiratory apparatus, showing various examples of different work of breathing alert or notification screens.

FIG. 26 illustrates an embodiment of a flow chart of a method of estimating a work of breathing (WOB) indicator and generating therapy parameter setting adjustment suggestions based on the generated WOB data.

FIGS. 27A and 27B illustrate schematic examples of a GUI display screen of the respiratory apparatus, showing various examples of a disconnection alert triggered in response to calculated WOB data.

FIGS. 28A and 28B illustrate schematic examples of a GUI display screen for adjusting the thresholds associated with notification, alerts and/or alarms triggered based on WOB indicators or data.

### DETAILED DESCRIPTION

Although certain examples are described below, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below.

### 1. Overview of Example Respiratory Apparatus

Examples of the method and/or processes for determining Work of Breathing (WOB), or indicators or parameters indicative of Work of Breathing, will be described in the context of an example respiratory apparatus 10 that is configured or operable to provide nasal high flow therapy via a unsealed patient interface. This is intended as a non-limiting example. It will be appreciated that the methods and processes may be applied to other respiratory apparatus or systems and/or to other modes of operation and/or modes of therapy delivered by such apparatus.

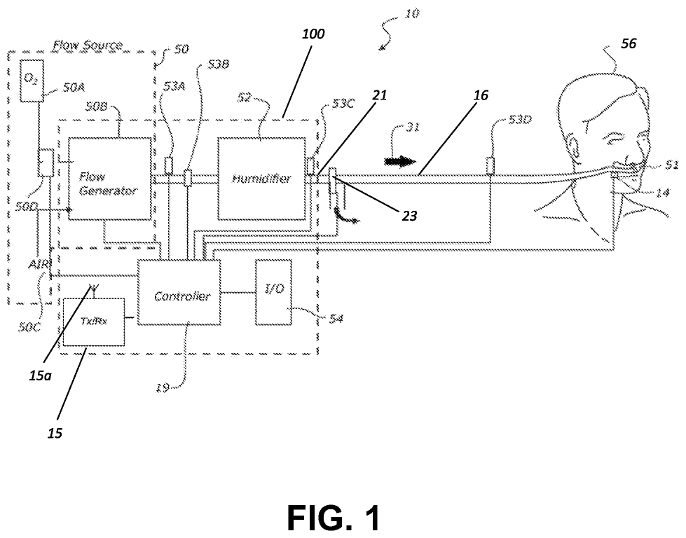

A schematic representation of the example respiratory apparatus 10 is provided in FIG. 1.

The respiratory apparatus 10 (or ‘respiratory system’) comprises a flow source 50 for providing a high flow gas 31 such as air, oxygen, air blended with oxygen, or a mix of air and/or oxygen and one or more other gases. Alternatively, the breathing assistance apparatus can have a connection for coupling to a flow source. As such, the flow source might be considered to form part of the apparatus or be separate to it, depending on context, or even part of the flow source forms part of the apparatus, and part of the flow source falls outside of the apparatus. In short, depending on the configuration (some components may be optional), the system can include a combination of components selected from the following:
a flow source,
humidifier for humidifying the gas-flow,
conduit (e.g., dry line or heated breathing tube),
patient interface,
non-return valve,
filter

The apparatus or system will now be described in more detail.

The flow source could be an in-wall supply of oxygen, a tank of oxygen 50A, a tank of other gas and/or a high flow apparatus with a flow generator 50B. FIG. 1 shows a flow source 50 with a flow generator 50B, with an optional air inlet 50C and optional connection to an O2 source (such as tank or O2 generator) 50A via a shut off valve and/or regulator and/or other gas flow control 50D, but this is just one option. The flow generator 50B can control flows delivered to the patient 56 using one or more valve, or optionally the flow generator 50B can comprise a blower. The flow source could be one or a combination of a flow generator 50B, O2 source 50A, air source 50C as described. The flow source 50 is shown as part of the apparatus 10, although in the case of an external oxygen tank or in-wall source, it may be considered a separate component, in which case the apparatus has a connection port to connect to such flow source. The flow source provides a (preferably high) flow of gas that can be delivered to a patient via a delivery conduit 16, and patient interface 51.

The patient interface 51 may be an unsealed (non-sealing) interface (for example when used in high flow therapy) such as a non-sealing nasal cannula, or a sealed (sealing) interface (for example when used in CPAP) such as a nasal mask, full face mask, or nasal pillows. In some embodiments, the patient interface 51 is a non-sealing patient interface which would for example help to prevent barotrauma (e.g., tissue damage to the lungs or other organs of the respiratory system due to difference in pressure relative to the atmosphere). In some embodiments, the patient interface 51 is a sealing mask that seals with the patient's nose and/or mouth. The patient interface may be a nasal cannula with a manifold and nasal prongs, and/or a face mask, and/or a nasal pillows mask, and/or a nasal mask, and/or a tracheostomy interface, or any other suitable type of patient interface. The flow source could provide a base gas flow rate of between, e.g., 0.5 litres/min and 375 litres/min, or any range within that range, or even ranges with higher or lower limits. Details of the ranges and nature of flow rates will be described later.

A humidifier 52 can optionally be provided between the flow source 50 and the patient to provide humidification of the delivered gas. One or more sensors 53A, 53B, 53C, 53D such as flow, oxygen fraction, pressure, humidity, temperature or other sensors can be placed throughout the system and/or at, on or near the patient 56. Alternatively, or additionally, sensors from which such parameters can be derived could be used. In addition, or alternatively, the sensors 53A-53D can be one or more physiological sensors for sensing patient physiological parameters such as, heart rate, oxygen saturation, partial pressure of oxygen in the blood, respiratory rate, partial pressure of CO2 in the blood. Alternatively, or additionally, sensors from which such parameters can be derived could be used. Other patient sensors could comprise EEG sensors, torso bands to detect breathing, and any other suitable sensors. In some configurations the humidifier may be optional, or it may be preferred due to the advantages of humidified gases helping to maintain the condition of the airways. One or more of the sensors might form part of the apparatus, or be external thereto, with the apparatus having inputs for any external sensors. The sensors can be coupled to or send their output to a controller 19.

In some configurations, the respiratory system 10 can include a sensor 14 for measuring the oxygen fraction of air the patient inspires. In some examples, the sensor 14 can be placed on the patient interface 51, to measure or otherwise determine the fraction of oxygen proximate (at/near/close to) the patient's mouth and/or nose. In some configurations, the output from the sensor 14 is sent to a controller 19 to assist control of the respiratory system 10 alter operation accordingly. The controller 19 is coupled to the flow source 50, humidifier 52 and sensor 14. In some configurations, the controller 19 controls these and other aspects of the respiratory system 10 as described herein. In some examples, the controller can operate the flow source 50 to provide the delivered flow of gas at a desired flow rate high enough to meet or exceed a user's (i.e., patient's) inspiratory demand. The flow rate is provided is sufficient that ambient gases are not entrained as the user (i.e., patient) inspires. In some configurations, the sensor 14 can convey measurements of oxygen fraction at the patient mouth and/or nose to a user, who can input the information to the respiratory system 10/controller 19.

An optional non-return valve 23 may be provided in the breathing conduit 16. A filter or filters may be provided at the air inlet 50C and/or inlets to the flow generator 50B to filter the incoming gases before they are pressurized into a high flow gas 31 by to the flow generator 50B.

The breathing assistance apparatus 10 could be an integrated or a separate component-based arrangement, generally shown in the dotted box 100 in FIG. 1. In some configurations, the apparatus or system could be a modular arrangement of components. Furthermore, the apparatus or system may just comprise some of the components shown, not necessarily all are essential. Also, the conduit and patient interface do not have to be part of the system, and could be considered separate. Hereinafter it will be referred to as a breathing assistance apparatus or respiratory system, but this should not be considered limiting. Breathing assistance apparatus and respiratory system will be broadly considered herein to comprise anything that provides a flow rate of gas to a patient. Some such apparatus and systems include a detection system that can be used to determine if the flow rate of gas meets inspiratory demand.

The respiratory apparatus 10 can include a main device housing 100. The main device housing 100 can contain the flow generator 50B that can be in the form of a motor/impeller arrangement, an optional humidifier or humidification chamber 52, a controller 19, and an input/output I/O user interface 54. The user interface 54 can include a display and input device(s) such as button(s), a touch screen (e.g., an LCD screen), a combination of a touch screen and button(s), or the like. The controller 19 can include one or more hardware and/or software processors and can be configured or programmed to control the components of the system, including but not limited to operating the flow generator 50B to create a flow of gases for delivery to a patient, operating the humidifier or humidification chamber 52 (if present) to humidify and/or heat the gases flow, receiving user input from the user interface 54 for reconfiguration and/or user-defined operation of the respiratory apparatus 10, and outputting information (for example on the display) to the user. The user can be a patient, healthcare professional, or others.

In one configuration, the user interface 54 of respiratory apparatus 10 may comprise a removable display screen or touch screen.

With continued reference to FIG. 1, a patient breathing conduit 16 can be coupled to a gases flow outlet (gases outlet or patient outlet port) 21 in the main device housing 100 of the respiratory apparatus 10, and be coupled to a patient interface 17, such as a non-sealing interface like a nasal cannula with a manifold and nasal prongs. The patient breathing conduit 16 can also be a tracheostomy interface, or other unsealed interfaces.

The gases flow can be generated by the flow generator 50B, and may be humidified, before being delivered to the patient via the patient breathing conduit 16 through the patient interface 51. The controller 19 can control the flow generator 50B to generate a gases flow of a desired flow rate, and/or one or more valves to control mixing of air and oxygen or other breathable gas. The controller 19 can control a heating element in or associated with the humidification chamber 52, if present, to heat the gases to a desired temperature that achieves a desired level of temperature and/or humidity for delivery to the patient. The patient breathing conduit 16 can have a heating element, such as a heater wire, to heat gases flow passing through to the patient. The heating element can also be under the control of the controller 19.

The humidifier 52 of the apparatus is configured to combine or introduce humidity with or into the gases flow. Various humidifier 52 configurations may be employed. In one configuration, the humidifier 52 can comprise a humidification chamber that is removable. For example, the humidification chamber may be partially or entirely removed or disconnected from the flow path and/or apparatus. By way of example, the humidification chamber may be removed for refilling, cleaning, replacement and/or repair for example. In one configuration, the humidification chamber may be received and retained by or within a humidification compartment or bay of the apparatus, or may otherwise couple onto or within the housing of the apparatus.

The humidification chamber of the humidifier 52 may comprise a gases inlet and a gases outlet to enable connection into the gases flow path of the apparatus. For example, the flow of gases from the flow generator 50B is received into the humidification chamber via its gases inlet and exits the chamber via its gases outlet, after being heated and/or humidified.

The humidification chamber contains a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a pass-over humidifier. In another configuration, the humidifier may be a non-pass-over humidifier.

In one configuration, the humidifier may comprise a heater plate, for example associated or within a humidification bay that the chamber sits on for heating. The chamber may be provided with a heat transfer surface, e.g, a metal insert, plate or similar, in the base or other surface of the chamber that interfaces or engages with the heater plate of the humidifier.

In another configuration, the humidification chamber may comprise an internal heater or heater elements inside or within the chamber. The internal heater or heater elements may be integrally mounted or provided inside the chamber, or may be removable from the chamber.

The humidification chamber may be any suitable shape and/or size. The location, number, size, and/or shape of the gases inlet and gases outlet of the chamber may be varied as required. In one configuration, the humidification chamber may have a base surface, one or more side walls extending up from the base surface, and an upper or top surface. In one configuration, the gases inlet and gases outlet may be position on the same side of the chamber. In another configuration, the gases inlet and gases outlet may be on different surfaces of the chamber, such as on opposite sides or locations, or other different locations.

In some configurations, the gases inlet and gases outlet may have parallel flow axes. In some configurations, the gases inlet and gases outlet may be positioned at the same height on the chamber.

The apparatus 10 can use ultrasonic transducer(s), flow sensor(s) such as a thermistor flow sensor, pressure sensor(s), temperature sensor(s), humidity sensor(s), or other sensors, in communication with the controller 19, to monitor characteristics of the gases flow and/or operate the system 10 in a manner that provides suitable therapy. The gases flow characteristics can include gases concentration, flow rate, pressure, temperature, humidity, or others. The sensors 53A, 53B, 53C, 53D, 14, such as pressure, temperature, humidity, and/or flow sensors, can be placed in various locations in the main device housing 100, the patient conduit 16, and/or the patient interface 51. The controller 19 can receive output from the sensors to assist it in operating the respiratory apparatus 10 in a manner that provides suitable therapy, such as to determine a suitable target temperature, flow rate, and/or pressure of the gases flow. Providing suitable therapy can include meeting or exceeding a patient's inspiratory demand. In the illustrated embodiment sensors 53A, 53B, and 53C are positioned in the housing of the apparatus, sensor 53D in the patient conduit 16, and sensor 14 in the patient interface 51.

The apparatus 10 can include one or more communication modules to enable data communication or connection with one or more external devices or servers over a data or communication link or data network, whether wired, wireless or a combination thereof. In one configuration for example, the apparatus 10 can include a wireless data transmitter and/or receiver, or a transceiver 15 to enable the controller 19 to receive data signals in a wireless manner from the operation sensors and/or to control the various components of the system 10. The transceiver 15 or data transmitter and/or receiver module may have an antenna 15a as shown. In one example, the transceiver may comprise a Wi-Fi modem. Additionally, or alternatively, the data transmitter and/or receiver 15 can deliver data to a remote patient management system (i.e., remote server) or enable remote control of the system 10. The system 10 can include a wired connection, for example, using cables or wires, to enable the controller 19 to receive data signals from the operation sensors and/or to control the various components of the apparatus 10. The apparatus 10 may comprise one or more wireless communication modules. For example, the apparatus may comprise a cellular communication module such as for example a 3G, 4G or 5G module. The module 15 may be or may comprise a modem that enables the apparatus to communicate with a remote patient management system (not illustrated in the figures) using an appropriate communication network. The remote management system may comprise a single server or multiple servers or multiple computing devices implemented in a cloud computing network. The communication may be two-way communication between the apparatus and a patient management system (e.g., a server) or other remote system. The apparatus 10 may also comprise other wireless communication modules such as for example a Bluetooth module and/or a Wi-Fi module. The Bluetooth and/or Wi-Fi module allow the apparatus to wirelessly send information to another device such as for example a smartphone or tablet or operate over a LAN (local area network) or Wireless LAN (WLAN). The apparatus may additionally, or alternatively, comprise a Near Field Communication (NFC) module to allow for data transfer and/or data communication.

For example, data representing determined or calculated work of breathing (WOB) indicators or values, or other associated WOB data (e.g. WOB trend data), or notification data (e.g. alerts, alarms, notifications) generated in response to WOB data may be communicated to a remote patient management system (i.e., a remote server) and/or to another remote electronic device (e.g., personal electronic device such as a smartphone, tablet, computer, laptop, wearable device). The remote patient management system may be a single server or a network of servers or a cloud computing system or other suitable architecture for operating a remote patient management system. The remote patient management system (i.e., remote server) further includes memory for storing received data and various software applications or services that are executed to perform multiple functions. Then, for example, the remote patient management system (i.e., remote server) may communicate information or instructions to the system 10 at least in part dependent on the data received. For example, the nature of the data received may trigger the remote server (or a software application running on the remote server) to communicate an alert, alarm, or notification to the system 10. The remote patient management system may further store the received data for access by an authorized party such as a clinician or the patient or another authorized party. The remote patient management system may further be configured to generate reports (e.g. report data, displayed reports, compiled reports, electronic reports, printable reports, numerical reports, graphical reports) in response to a request from an authorized party, and the work of breathing data or associated WOB data may be included into the generated reports. The reports may further comprise other data or patient breathing parameters e.g., respiratory rate or SpO2 and/or device parameters e.g. flow rate, oxygen concentration of the gas flow (e.g. sensed oxygen concentration and/or FiO2 and/or FdO2 parameter settings), humidity level, or other such device parameters.

The respiratory apparatus 10 may comprise a high flow therapy apparatus. High flow therapy as discussed herein is intended to be given its typical ordinary meaning, as understood by a person of skill in the art, which generally refers to a respiratory system delivering a targeted flow of humidified respiratory gases via an intentionally unsealed patient interface with flow rates generally intended to meet or exceed inspiratory flow of a user. Typical patient interfaces include, but are not limited to, a nasal or tracheal patient interface. Typical flow rates for adults often range from, but are not limited to, about fifteen litres per minute to about sixty litres per minute or greater. Typical flow rates for paediatric users (such as neonates, infants and children) often range from, but are not limited to, about one litre per minute per kilogram of user weight to about three litres per minute per kilogram of user weight or greater.

High flow therapy can also optionally include gas mixture compositions including supplemental oxygen and/or administration of therapeutic medicaments.

High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc. High flow therapy can also increase expiratory time of the patient due to pressure during expiration. This in turn reduces the respiratory rate of the patient.

The patient interface for use in a high flow therapy can be a non-sealing interface to prevent barotrauma, which can include tissue damage to the lungs or other organs of the patient's respiratory system due to difference in pressure relative to the atmosphere. The patient interface can be a nasal cannula with a manifold and nasal prongs, and/or an unsealed tracheostomy interface, or any other suitable type of patient interface.

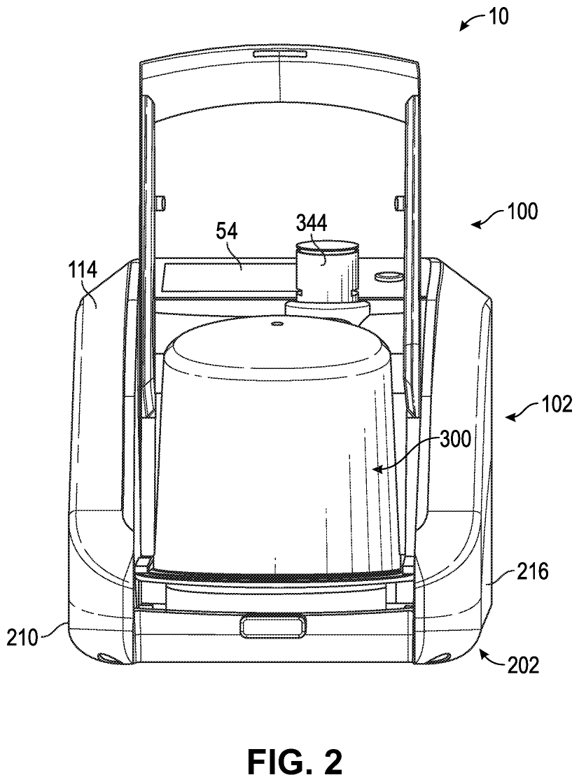

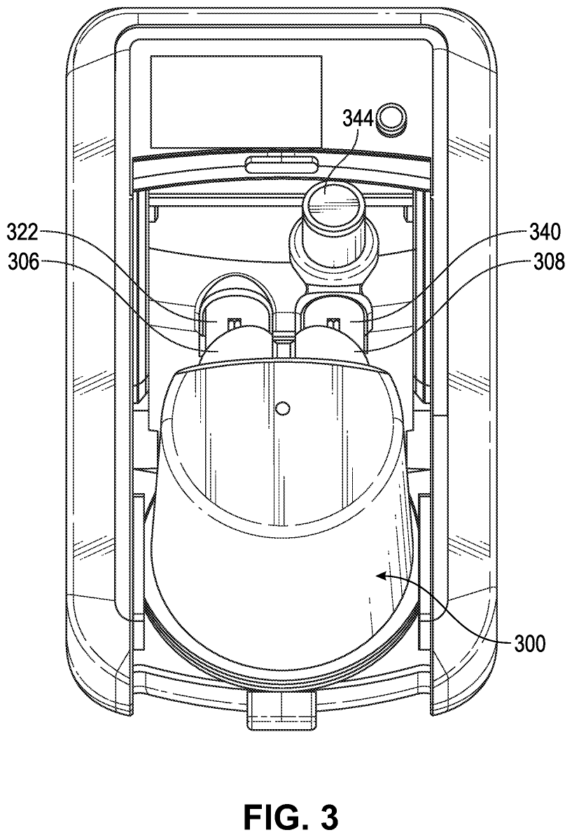

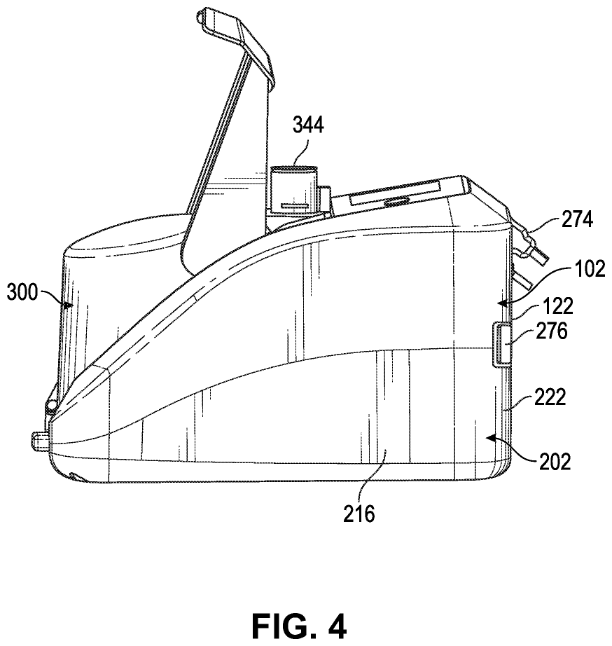

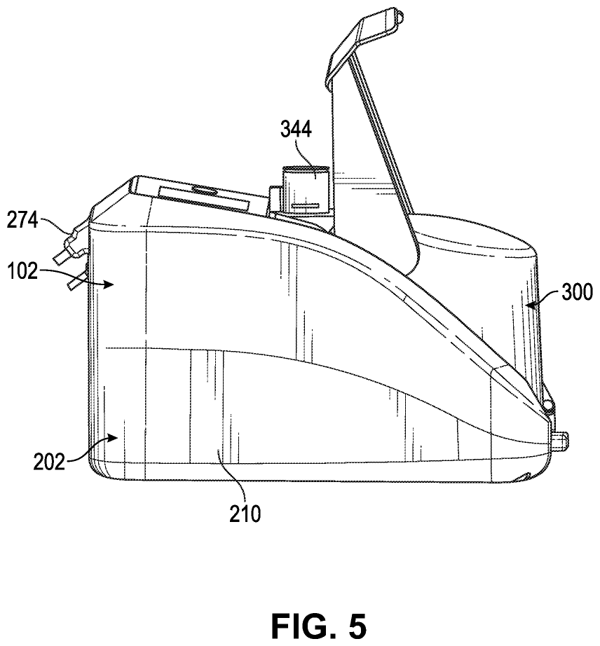

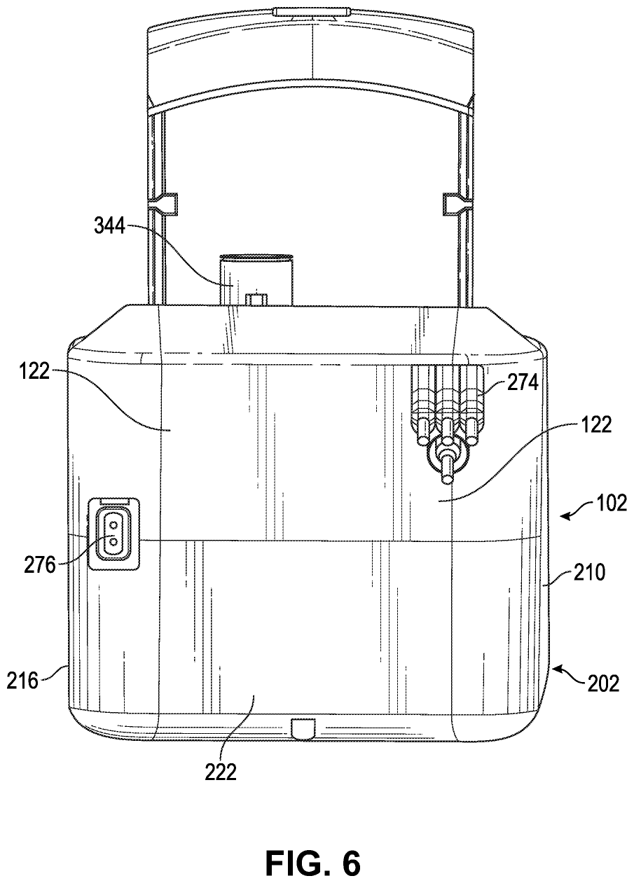

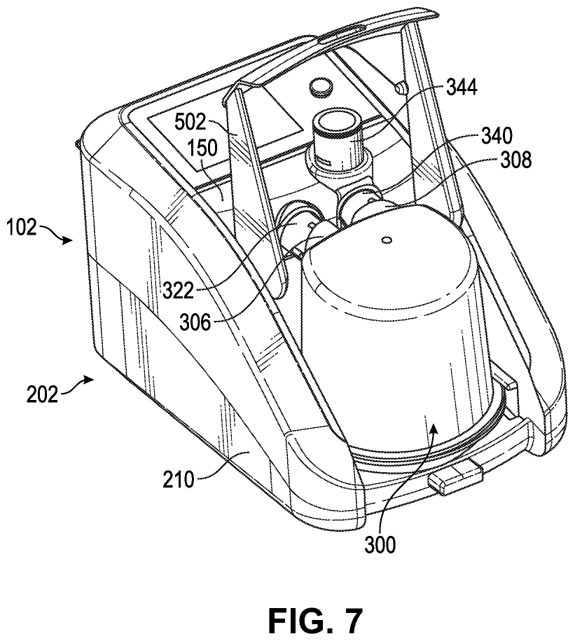

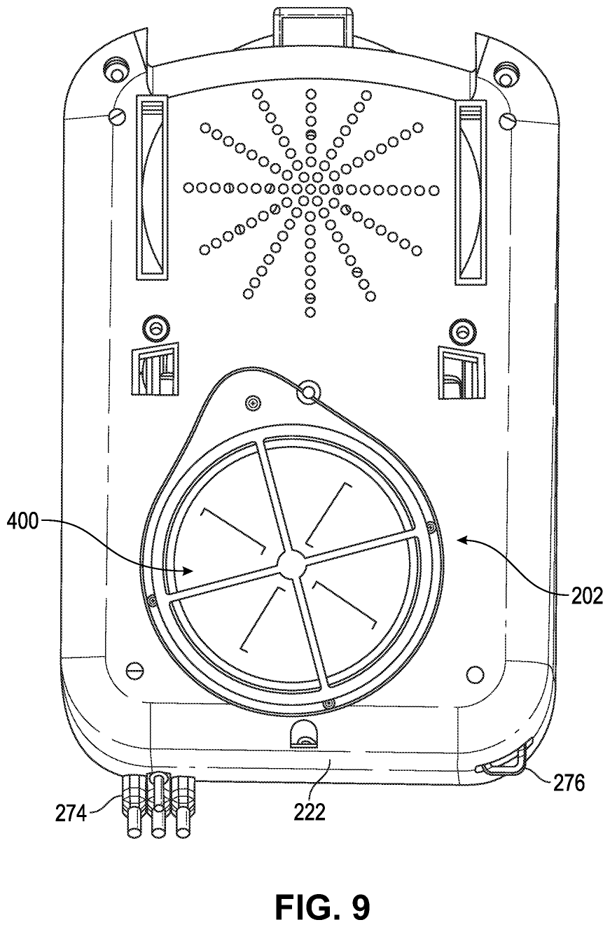

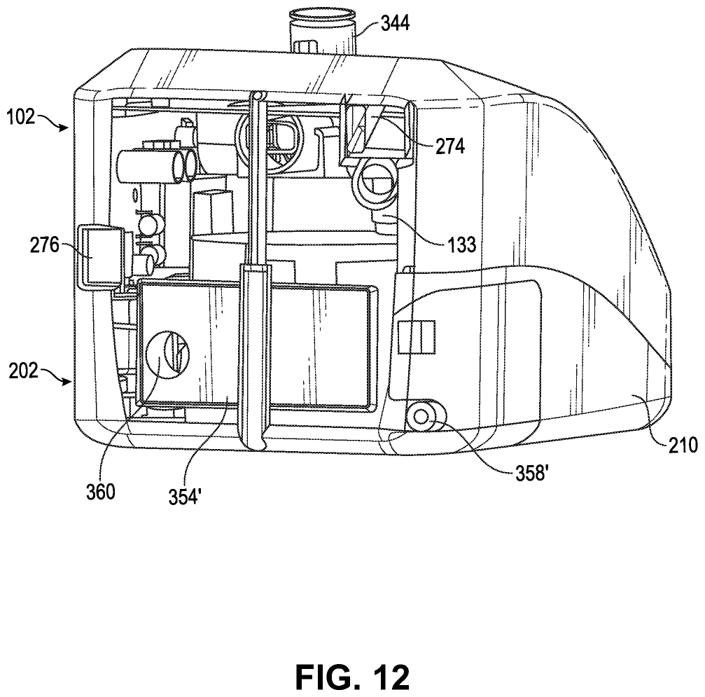

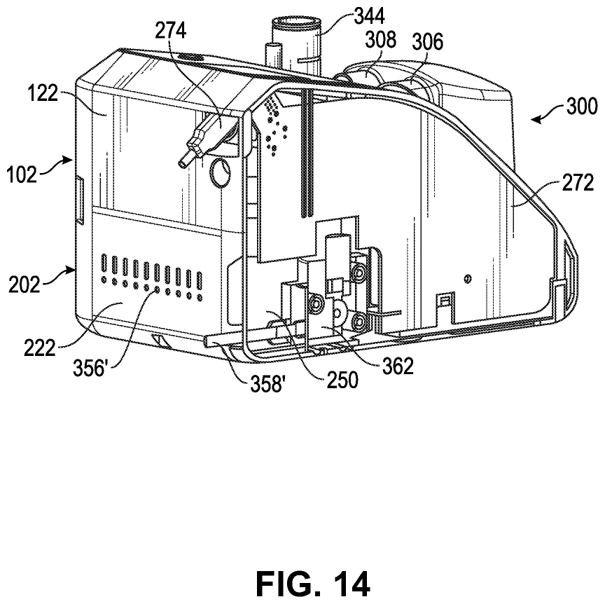

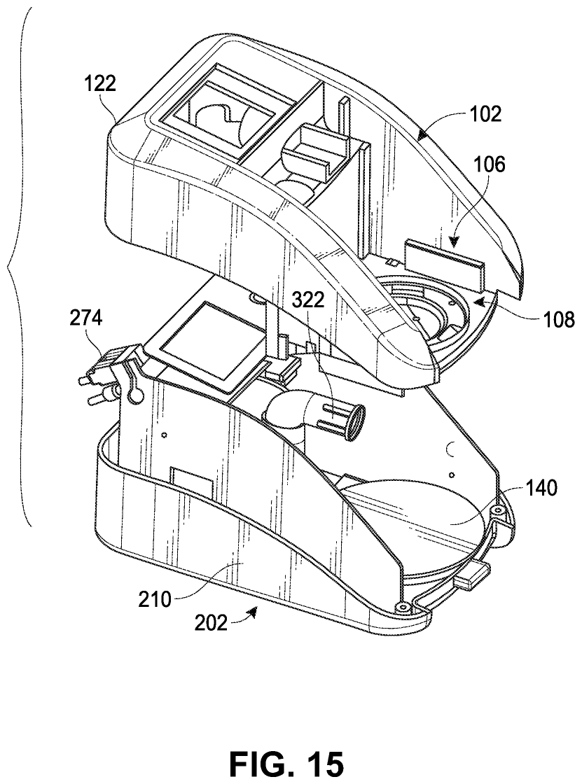

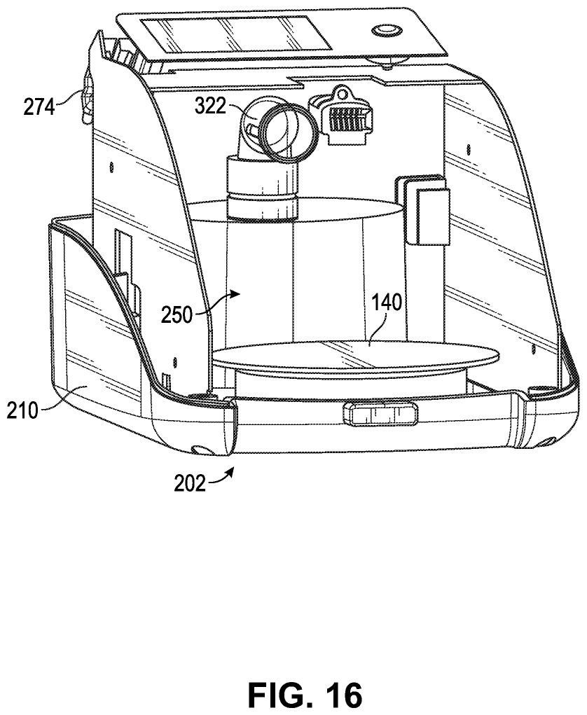

FIGS. 2 to 18 show an example respiratory device of the respiratory apparatus 10 having a main housing 100. The main housing 100 has a main housing upper chassis 102 and a main housing lower chassis 202. The main housing upper chassis 102 has a peripheral wall arrangement 106 (see FIG. 15). The peripheral wall arrangement defines a humidifier or humidification chamber bay 108 for receipt of a removable humidification chamber 300. The removable humidification chamber 300 contains a suitable liquid such as water for humidifying gases that can be delivered to a patient. A floor portion of the humidification chamber bay 108 can have a recess to receive a heater arrangement such as a heater plate 140 or other suitable heating element(s) for heating liquid in the humidification chamber 300 for use during a humidification process.

The humidification chamber 300 can be fluidly coupled to the apparatus 10 in a linear slide-on motion in a rearward direction of the humidification chamber 300 into the chamber bay 108, from a position at the front of the housing 100 in a direction toward the rear of the housing 100. A gases outlet port 322 can be in fluid communication with the motor.

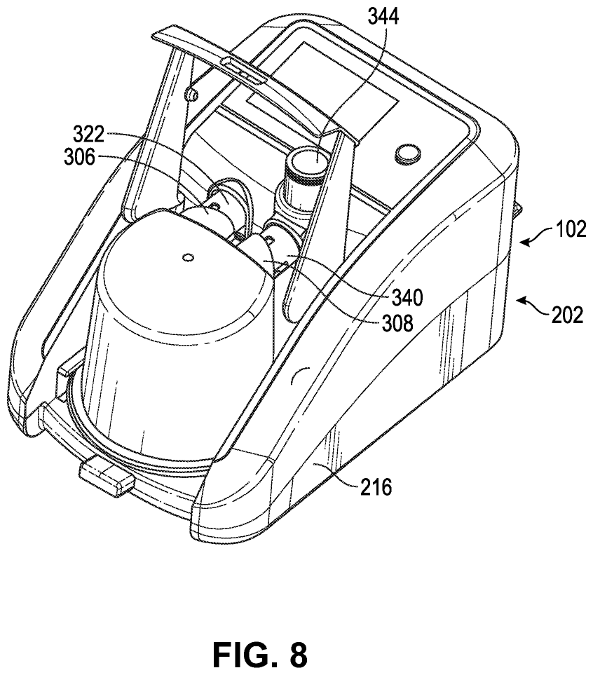

A gases inlet port 340 (humidified gases return) as shown in FIG. 8 can include a removable L-shaped elbow. The removable elbow can further include a patient outlet port 344 for coupling to the patient conduit 16 to deliver gases to the patient interface. The gases outlet port 322, gases inlet port 340, and patient outlet port 344 each can have soft seals such as O-ring seals or T-seals to provide a sealed gases passageway between the apparatus 10, the humidification chamber 300, and the patient conduit 16.

The humidification chamber gases inlet port 306 can be complementary with the gases outlet port 322, and the humidification chamber gases outlet port 308 can be complementary with the gases inlet port 340. The axes of those ports can be parallel to each other to enable the humidification chamber 300 to be inserted into the chamber bay 108 in a linear movement.

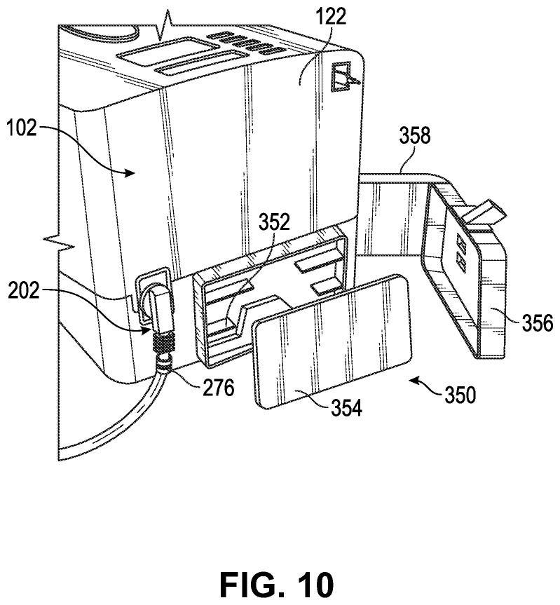

The respiratory device can have air and oxygen (or alternative auxiliary gas) inlets in fluid communication with the motor to enable the motor to deliver air, oxygen (or alternative auxiliary gas), or a mixture thereof to the humidification chamber 300 and thereby to the patient. As shown in FIG. 10, the device can have a combined air/oxygen (or alternative auxiliary gas) inlet arrangement 350. This arrangement can include a combined air/oxygen port 352 into the housing 100, a filter 354, and a cover 356 with a hinge 358. A gases tube can also optionally extend laterally or in another appropriate direction and be in fluid communication with an oxygen (or alternative auxiliary gas) source. The port 352 can be fluidly coupled with the motor 402. For example, the port 352 may be coupled with the motor/sensor module 400 via a gases flow passage between the port 352 and an inlet aperture or port in the motor and sensor module 400, which in turn would lead to the motor.

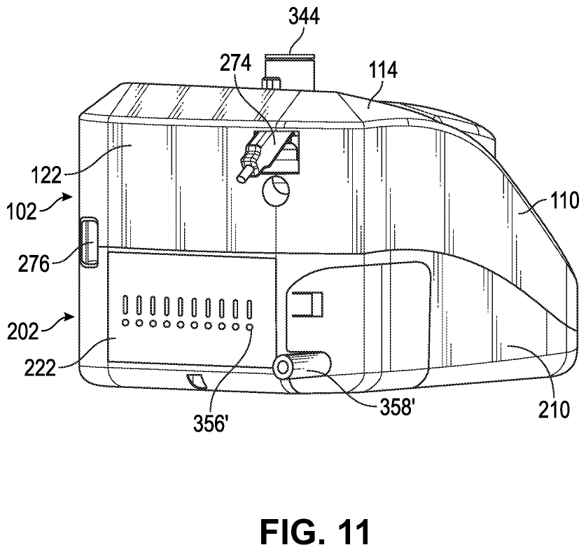

The device can have the arrangement shown in FIGS. 11 to 14 to enable the blower to deliver air, oxygen (or alternative auxiliary gas), or a suitable mixture thereof to the humidification chamber 300 and thereby to the patient. This arrangement can include an air inlet 356′ in the rear wall 222 of the lower chassis 202 of the housing 100. The air inlet 356′ comprises a rigid plate with a suitable grill arrangement of apertures and/or slots. Sound dampening foam may be provided adjacent the plate on the interior side of the plate. An air filter box 354′ can be positioned adjacent the air inlet 356′ internally in the main housing 100, and include an air outlet port 360 to deliver filtered air to the motor via an air inlet port 404 in the motor/sensor module 400. The air filter box 354′ may include a filter configured to remove particulates (e.g., dust) and/or pathogens (e.g., viruses or bacteria) from the gases flow. A soft seal such as an O-ring seal can be provided between the air outlet port 360 and air inlet port 404 to seal between the components. The device can include a separate oxygen inlet port 358′ positioned adjacent one side of the housing 100 at a rear end thereof, the oxygen port 358′ for receipt of oxygen from an oxygen source such as a tank or source of piped oxygen. The oxygen inlet port 358′ is in fluid communication with a valve 362. The valve 362 can suitably be a solenoid valve that enables the control of the amount of oxygen that is added to the gases flow that is delivered to the humidification chamber 300. The oxygen port 358′ and valve 362 may be used with other auxiliary gases to control the addition of other auxiliary gases to the gases flow. The other auxiliary gases can include any one or more of a number of gases useful for gas therapy, including but not limited to heliox and nitric oxide.

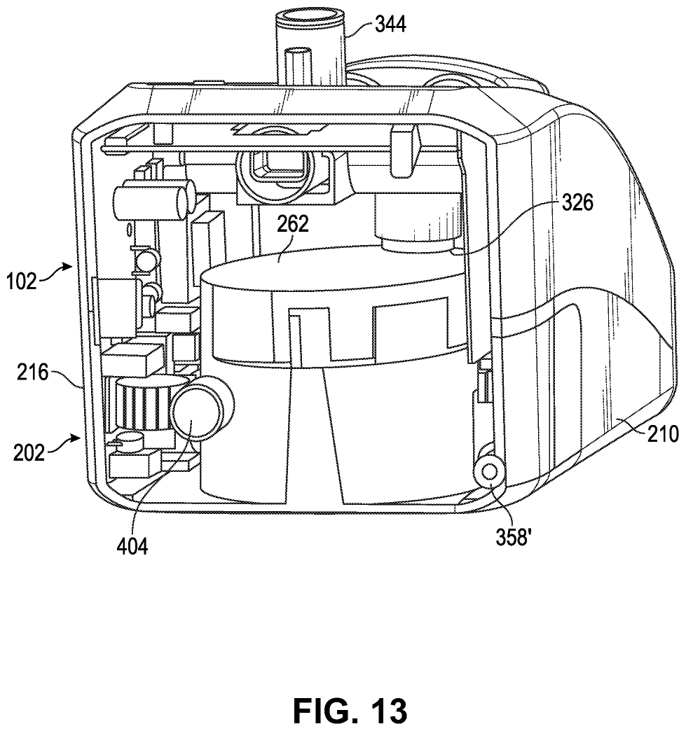

As shown in FIGS. 13 to 16, the lower housing chassis 202 can include suitable electronics boards, such as sensing circuit boards. The electronics boards can be positioned adjacent respective outer side walls 210, 216 of the lower housing chassis 202. The electronics boards can contain, or can be in electrical communication with, suitable electrical or electronics components, such as but not limited to microprocessors, capacitors, resistors, diodes, operational amplifiers, comparators, and switches. Sensors can be used with the electronic boards. Components of the electronics boards (such as but not limited to one or more microprocessors) can act as the controller 19 of the apparatus.

One or more of the electronics boards can be in electrical communication with the electrical components of the apparatus 10, including the display unit and user interface 54, motor, valve 362, and the heater plate 140 to operate the motor to provide the desired flow rate of gases, operate the humidification chamber 300 to humidify and heat the gases flow to an appropriate level, and supply appropriate quantities of oxygen (or quantities of an alternative auxiliary gas) to the gases flow.

The electronics boards can be in electrical communication with a connector arrangement 274 projecting from a rear wall 122 of the upper housing chassis 102. The connector arrangement 274 may be coupled to an alarm, pulse oximetry port, and/or other suitable accessories. The electronics boards can also be in electrical communication with an electrical connector 276 that can also be provided in the rear wall 122 of the upper housing chassis 102 to provide mains or battery power to the components of the device.

As mentioned above, operation sensors, such as flow, temperature, humidity, and/or pressure sensors can be placed in various locations in the respiratory device, the patient breathing conduit 16, and/or cannula 51 such as shown in FIG. 1. The electronics boards can be in electrical communication with those sensors. Output from the sensors can be received by the controller 19, to assist the controller 19 to operate the respiratory apparatus 10 in a manner that provides optimal therapy, for example controlling to a set flow rate. The set flow rate may be selected such that it provides flushing of the patient's upper airways and/or meets or exceeds a patient's inspiratory demand and/or provides other advantages of high flow therapy described herein. In the illustrated embodiment the sensors are positioned on electronic boards that are positioned within the housing. The sensors are encapsulated within the housing.

As outlined above, the electronics boards and other electrical and electronic components can be pneumatically isolated from the gases flow path to improve safety. The sealing also prevents water ingress.

### 1.1 Control System

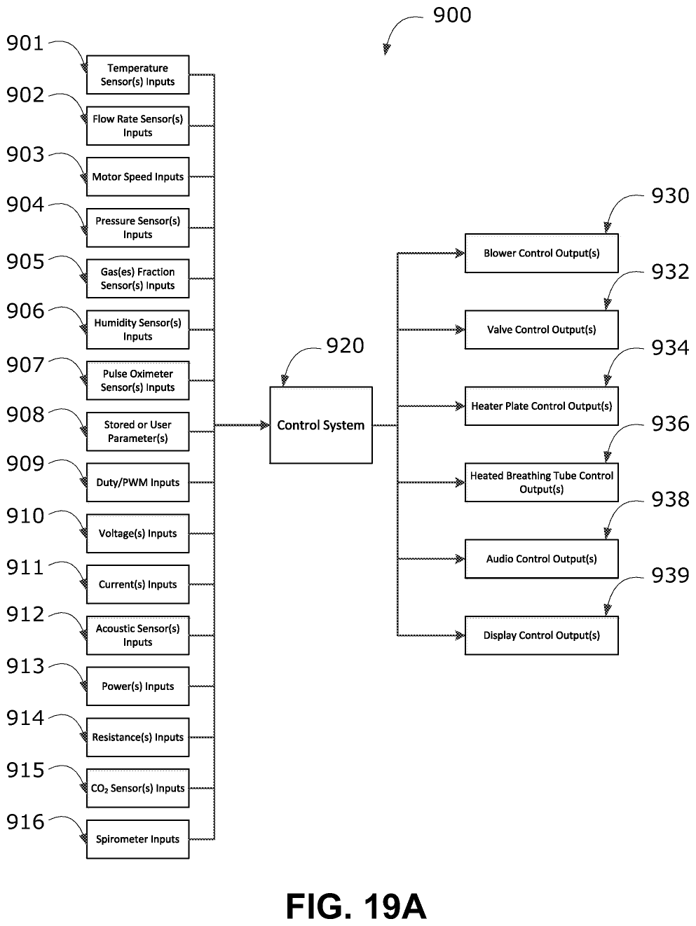

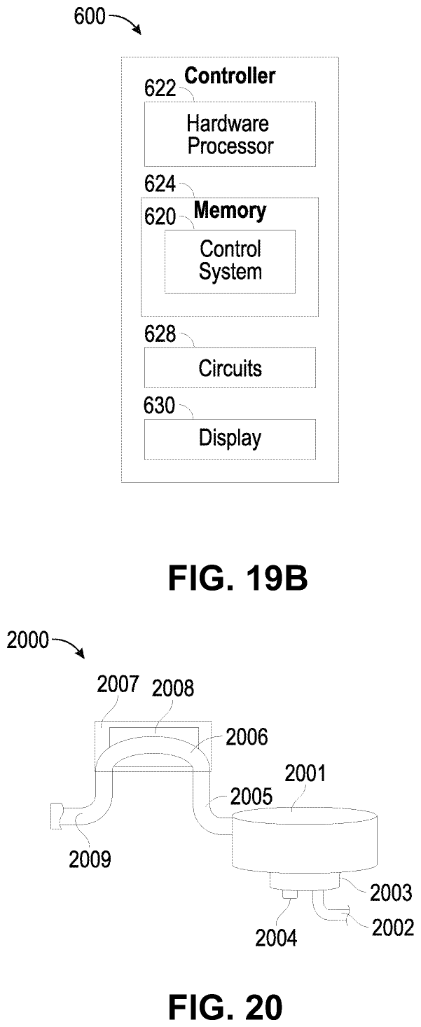

FIG. 19A illustrates a block diagram 900 of an example control system 920 (which can be the controller 19 in FIG. 1) that can detect patient conditions and control operation of the respiratory system including the gases source. The control system 920 can manage a flow rate of the gases flowing through the respiratory system as is the gases are delivered to a patient. For example, the control system 920 can increase or decrease the flow rate by controlling an output of a motor speed of the blower (hereinafter also referred to as a “blower motor”) 930 or an output of a valve 932 in a blender. The control system 920 can automatically determine a set value or a personalized value of the flow rate for a particular patient as discussed below. The flow rate can be optimized by the control system 920 to improve patient comfort and therapy.

The control system 920 can also generate audio and/or display/visual outputs 938, 939. For example, the flow therapy apparatus can include a display and/or an audio output device (e.g., speaker). The display can indicate to the physicians any warnings or alarms generated by the control system 920. The display can also indicate control parameters that can be adjusted by the physicians. For example, the control system 920 can automatically recommend a flow rate for a particular patient. The control system 920 can also determine a respiratory state of the patient, including but not limited to generating a respiratory rate of the patient, and send it to the display, which will be described in greater detail below.

The control system 920 can change heater control outputs to control one or more of the heating elements (for example, to maintain a temperature set point of the gases delivered to the patient). The control system 920 can also change the operation or duty cycle of the heating elements. The heater control outputs can include heater plate control output(s) 934 and heated breathing tube control output(s) 936.

The control system 920 can determine the outputs 930-939 based on one or more received inputs 901-916. The inputs 901-916 can correspond to sensor measurements received automatically by the controller 600 (shown in FIG. 19B). The control system 920 can receive sensor inputs including but not limited to temperature sensor(s) inputs 901, flow rate sensor(s) inputs 902, motor speed inputs 903, pressure sensor(s) inputs 904, gas(s) fraction sensor(s) inputs 905, humidity sensor(s) inputs 906, pulse oximeter (for example, SpO₂) sensor(s) inputs 907, stored or user parameter(s) 908, duty cycle or pulse width modulation (PWM) inputs 909, voltage(s) inputs 910, current(s) inputs 911, acoustic sensor(s) inputs 912, power(s) inputs 913, resistance(s) inputs 914, CO2 sensor(s) inputs 915, and/or spirometer inputs 916. The control system 920 can receive inputs from the user or stored parameter values in a memory 624 (shown in FIG. 19B). The control system 920 can dynamically adjust flow rate for a patient over the time of their therapy. The control system 920 can continuously detect system parameters and patient parameters. A person of ordinary skill in the art will appreciate based on the disclosure herein that any other suitable inputs and/or outputs can be used with the control system 920.

### 1.2 Controller

FIG. 19B illustrates a block diagram of an embodiment of a controller 600 (which can be the controller 19 in FIG. 1). The controller 600 can include programming instructions for detection of input conditions and control of output conditions. The programming instructions can be stored in the memory 624 of the controller 600. The programming instructions can correspond to the methods, processes and functions described herein. The programming instructions can be executed by one or more hardware processors 622 of the controller 600. The programming instructions can be implemented in C, C++, JAVA, or any other suitable programming languages. Some or all of the portions of the programming instructions can be implemented in application specific circuitry 628 such as ASICs and FPGAs.

The controller 600 can also include circuits 628 for receiving sensor signals. The controller 600 can further include a display 630 for transmitting status of the patient and the respiratory assistance system. The display 630 can also show warnings and/or other alerts. The display 630 can be configured to display characteristics of sensed gas(es) in real time or otherwise. The controller 600 can also receive user inputs via the user interface such as display 630. The user interface can include button(s) and/or dial(s). The user interface can comprise a touch screen.

### 1.3 Motor and Sensor Module

Any of the features of the respiratory system described herein, including but not limited to the humidification chamber, the flow generator, the user interface, the controller, and the patient breathing conduit configured to couple the gases flow outlet of the respiratory system to the patient interface, can be combined with any of the sensor modules described herein.

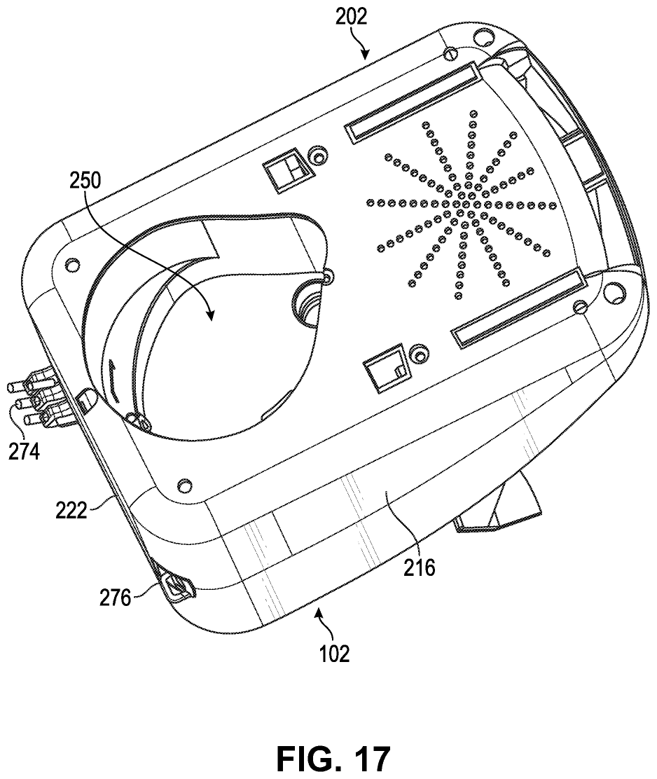

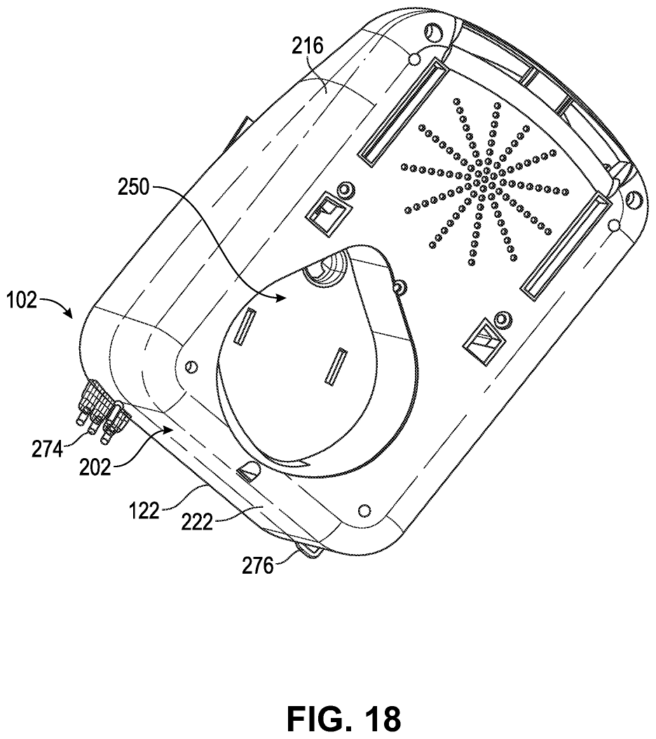

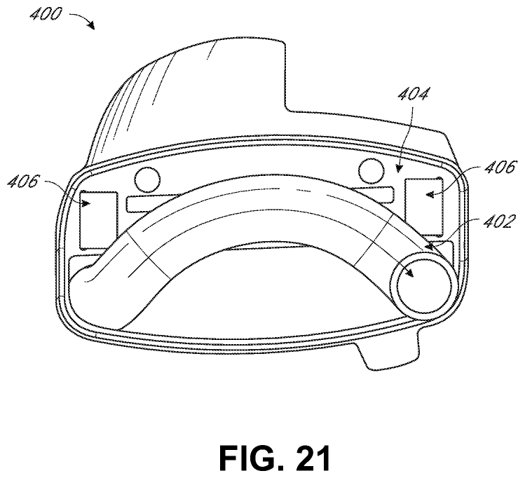

FIG. 20 illustrates a block diagram of the motor and sensor module 2000 (or ‘sensing block’), which can be received by the recess 250 in the respiratory device (shown in FIGS. 17 and 18). The motor and sensor module can include a blower 2001, which entrains room air to deliver to a patient. The blower 2001 can be a centrifugal blower.

One or more sensors (for example, Hall-effect sensors) may be used to measure a motor speed of the blower motor. The blower motor may comprise a brushless DC motor, from which motor speed can be measured without the use of separate sensors. For example, during operation of a brushless DC motor, back-EMF can be measured from the non-energized windings of the motor, from which a motor position can be determined, which can in turn be used to calculate a motor speed. In addition, a motor driver may be used to measure motor current, which can be used with the measured motor speed to calculate a motor torque. The blower motor may comprise a low inertia motor.

Room air can enter a room air inlet 2002, which enters the blower 2001 through an inlet port 2003. The inlet port 2003 can include a valve 2004 through which a pressurized gas may enter the blower 2001. The valve 2004 can control a flow of oxygen into the blower 2001. The valve 2004 can be any type of valve, including a proportional valve or a binary valve. In some embodiments, the inlet port does not include a valve.

The blower 2001 can operate at a motor speed of greater than 1,000 RPM and less than 30,000 RPM, greater than 2,000 RPM and less than 21,000 RPM, or between any of the foregoing values. Operation of the blower 2001 mixes the gases entering the blower 2001 through the inlet port 2003. Using the blower 2001 as the mixer can decrease the pressure drop that would otherwise occur in a system with a separate mixer, such as a static mixer comprising baffles, because mixing requires energy.

The mixed air can exit the blower 2001 through a conduit 2005 and enters the flow path 2006 in the sensor chamber 2007. A sensing circuit board with sensors 2008 can positioned in the sensor chamber 2007 such that the sensing circuit board is at least partially immersed in the gases flow. At least some of the sensors 2008 on the sensing circuit board can be positioned within the gases flow to measure gases properties within the flow. After passing through the flow path 2006 in the sensor chamber 2007, the gases can exit 2009 to the humidification chamber.

Positioning sensors 2008 downstream of the combined blower and mixer 2001 can increase accuracy of measurements, such as the measurement of gases fraction concentration, including oxygen concentration, over systems that position the sensors upstream of the blower and/or the mixer. Such a positioning can give a repeatable flow profile. Further, positioning the sensors downstream of the combined blower and mixer avoids the pressure drop that would otherwise occur, as where sensing occurs prior to the blower, a separate mixer, such as a static mixer with baffles, is required between the inlet and the sensing system. The mixer can introduce a pressure drop across the mixer. Positioning the sensing after the blower can allow the blower to be a mixer, and while a static mixer would lower pressure, in contrast, a blower increases pressure. Also, immersing at least part of the sensing circuit board and sensors 2008 in the flow path can increase the accuracy of measurements because the sensors being immersed in the flow means they are more likely to be subject to the same conditions, such as temperature and pressure, as the gases flow and therefore provide a better representation of the gases flow characteristics.

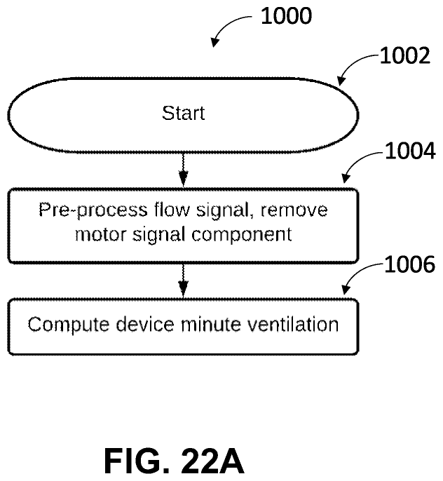

Referring to FIG. 21, the gases exiting the blower can enter a flow path 402 in a sensor chamber 400, which can be positioned within the motor and sensor module and can be the sensor chamber 2007 of FIG. 20. The flow path 402 can have a curved shape. The flow path 402 can be configured to have a curved shape with no sharp turns. The flow path 402 can have curved ends with a straighter section between the curved ends. A curved flow path shape can reduce pressure drop in a gases flow without reducing the sensitivity of flow measurements by partially coinciding a measuring region with the flow path to form a measurement portion of the flow path.

A sensing circuit board 404 with sensors, such as acoustic transmitters and/or receivers, humidity sensor, temperature sensor, thermistor, and the like, can be positioned in the sensor chamber 400 such that the sensing circuit board 404 is at least partially immersed in the flow path 402. Immersing at least part of the sensing circuit board and sensors in the flow path can increase the accuracy of measurements because the sensors immersed in the flow are more likely to be subject to the same conditions, such as temperature and pressure, as the gases flow, and therefore provide a better representation of the characteristics of the gases flow. After passing through the flow path 402 in the sensor chamber 400, the gases can exit to the humidification chamber.

The gases flow rate may be measured using at least two different types of sensors. The first type of sensor can comprise a thermistor, which can determine a flow rate by monitoring heat transfer between the gases flow and the thermistor. The thermistor flow sensor can run the thermistor at a constant target temperature within the flow when the gases flow around and past the thermistor. The sensor can measure an amount of power required to maintain the thermistor at the target temperature. The target temperature can be configured to be higher than a temperature of the gases flow, such that more power is required to maintain the thermistor at the target temperature at a higher flow rate.

The thermistor flow rate sensor can also maintain a plurality of (for example, two, three, or more) constant temperatures on a thermistor to avoid the difference between the target temperature and the gases flow temperature from being too small or too large. The plurality of different target temperatures can allow the thermistor flow rate sensor to be accurate across a large temperature range of the gases. For example, the thermistor circuit can be configured to be able to switch between two different target temperatures, such that the temperature of the gases flow will always fall within a certain range relative to one of the two target temperatures (for example, not too close but not too far). The thermistor circuit can be configured to operate at a first target temperature of about 50° C. to about 70° C., or about 66° C. The first target temperature can be associated with a desirable flow temperature range of between about 0° C. to about 60° C., or about 0° C. and about 40° C. The thermistor circuit can be configured to operate at a second target temperature of about 90° C. to about 110° C., or about 100° C. The second target temperature can be associated with a desirable flow temperature range of between about 20° C. to about 100° C., or about 30° C. and about 70° C.

The controller can be configured to adjust the thermistor circuit to change between at least the first and second target temperature modes by connecting or bypassing a resistor within the thermistor circuit. The thermistor circuit can be arranged as a Wheatstone bridge configuration comprising a first voltage divider arm and a second voltage divider arm. The thermistor can be located on one of the voltage divider arms. More details of a thermistor flow rate sensor are described in PCT Application Publication No. WO2018/052320, filed 3 Sep. 2017, which is incorporated by reference herein in its entirety.

The second type of sensor can comprise an acoustic sensor assembly. Acoustic sensors including acoustic transmitters and/or receivers can be used to measure a time of flight of acoustic signals to determine gases velocity and/or composition, which can be used in flow therapy apparatuses. In one ultrasonic sensing (including ultrasonic transmitters and/or receivers) topology, a driver causes a first sensor, such as an ultrasonic transducer, to produce an ultrasonic pulse in a first direction. A second sensor, such as a second ultrasonic transducer, receives this pulse and provides a measurement of the time of flight of the pulse between the first and second ultrasonic transducers. Using this time-of-flight measurement, the speed of sound of the gases flow between the ultrasonic transducers can be calculated by a processor or controller of the respiratory system. The second sensor can transmit, and the first sensor can receive a pulse in a second direction opposite the first direction to provide a second measurement of the time of flight, allowing characteristics of the gases flow, such as a flow rate or velocity, to be determined. In another acoustic sensing topology, acoustic pulses transmitted by an acoustic transmitter, such as an ultrasonic transducer, can be received by acoustic receivers, such as microphones. More details of an acoustic flow rate sensor are described in PCT Application Publication No. WO2017/095241, filed 2 Dec. 2016, which is incorporated by reference herein in its entirety.

The one or more flow rate sensors, or a sensor assembly comprising a flow rate sensor or sensors, may be located in various positions in the respiratory apparatus and/or along the gases flow path. In one configuration, a flow rate sensor or sensors, or a sensory assembly, may be located or arranged after the flow generator 50B, i.e., the sensor is configured or arranged to sense or measure the flow rate of the gases in the flow path after the flow generator 50B. In this configuration, the flow rate signal or flow rate data generated by the flow rate sensor or sensors may represent the flow generator output flow rate signal or data, i.e., the flow rate of the gases flow output from the flow generator 50B.

In one example configuration, a flow rate sensor or sensors, or sensor assembly, may be located in the main device housing 100 before or after the humidifier 52 (if present). For example, the flow rate sensor may be arranged or configured in the main device housing 100 to sense the flow rate of the gases in the flow path at a location between the flow generator 50B and humidifier 52, or a location in the flow path after the humidifier. In another example configuration, a flow rate sensor or sensors, or sensor assembly, may be located in or along the breathing conduit 16 and/or patient interface 51. In this configuration, the sensor or sensor assembly is configured to sense or measure the flow rate of the gases flow in the flow path comprising or formed by the breathing conduit 16 and/or patient interface 51, i.e., the flow path that follows the gases flow outlet 21 of the main device housing 100. In another example configuration, the apparatus may comprise any combination of the mentioned one or more flow rate sensor or sensor assembly configurations or locations. For example, the apparatus may comprise any combination of one or more flow rate sensors or sensor assemblies in any one or more locations along the gases flow path, whether in the main device housing 100, breathing conduit 16, and/or patient interface 51.

In some configurations, readings from both the first and second types of sensors can be combined to determine a more accurate flow measurement. For example, a previously determined flow rate and one or more outputs from one of the types of sensor can be used to determine a predicted current flow rate. The predicted current flow rate can then be updated using one or more outputs from the other one of the first and second types of sensor, in order to calculate a final flow rate.

### 2. Example Embodiments of Work of Breathing Determination Processes

The methods and processes of determining data indicative of or representing work of breathing will be described in the context of the example respiratory apparatus 10 described above, which is configured or operable to provide nasal high flow therapy via an unsealed patient interface. As explained earlier, the methods and processes may also be applied to other respiratory apparatus and/or to other modes of operation and/or modes of therapy delivered by such apparatus.

### 2.1 Summary of Work of Breathing (WOB) Determination Process

Work of breathing (WOB) is a clinical metric which provides a valuable indicator of respiratory therapy efficacy. Determining estimates or indicators of WOB can be used to improve patient outcomes when using a respiratory apparatus. For example, in some configurations, the WOB indicators can be used to assess whether present respiratory apparatus and/or therapy settings need to be adjusted, which parameters to adjust, and/or by how much to adjust the settings.

As will be appreciated by the skilled person, WOB has a generally accepted definition relating to the energy or work needed or exerted by a person to breathe. One way to determine true WOB is with a chest band that measures force and depth of chest movement as a person breathes, but these are not always convenient or practical for some patients. Respiratory rate may be used as an indicator of WOB in some scenarios. This disclosure provides methods and processes for determining one or more alternative analogues, surrogates or indicators of WOB for a patient while they are undergoing high flow therapy delivered by a respiratory apparatus. The alternative WOB indicators may be used to enhance clinical decision making, patient outcomes, and/or operation of the respiratory apparatus to deliver improved high flow therapy.

The embodiments described below aim to provide a method for reliably estimating patient WOB indicators using sensor data available from the respiratory apparatus and/or algorithms implemented thereon.

This disclosure relates to methods and/or algorithms for determining one or more patient or user WOB indicators or estimates based at least partly on sensed flow parameter data indicative or representative of the flow of gases in the respiratory apparatus during use by a patient or user. By way of example, methods for determining three different user WOB indicators or estimates will be described below. Each of the three example methods involve estimation of ΔPₙₒₛₑ, which represents a nasal pressure variation value indicative of the user's average nasal pressure when they are undergoing respiratory therapy with the respiratory apparatus.

In the example methods, the various WOB indicators are determined or calculated based at least partly on flow parameter data indicative or representative of the flow of gases in the flow path of the respiratory apparatus.

In an example configuration, the flow parameter data includes flow rate data indicative or representative of the flow rate of the flow of gases provided by the respiratory apparatus during respiratory therapy. In one example, the flow rate data may be a flow signal generated by a flow sensor or sensors provided or positioned in the flow path of the respiratory apparatus. The flow sensor or sensors may be provided in the flow path downstream of the flow generator of the respiratory apparatus. Fluctuations in the flow rate signal around typical human breathing frequencies will be observed when a patient is using the respiratory apparatus during respiratory therapy. The flow rate signal may be very noisy, and in the example configurations, algorithms and/or signal pre-processing may be applied to the raw flow signal generated by the flow sensor(s) to minimise the effects of noise. By analysing fluctuations in the pre-processed flow rate signal, various useful WOB parameters or indicators associated with a patient can be extracted while they are undergoing respiratory therapy with the respiratory apparatus.

In some example configurations, the flow parameter data used to generate the WOB indicators may additionally or alternatively include pressure data indicative or representative of the flow of gases provided by the flow generator (e.g. the pressure at the outlet of the blower in the respiratory apparatus).

As will be seen, in these example configurations, there are some common steps to determining all the three example WOB indicators, which will be outlined below.

The methods and/or algorithms for generating the WOB indicators may be executed or implemented on any suitable controller or processor. In the example configuration, the methods and/or algorithms for generating the WOB indicators may be executed or implemented on the main or primary controller of the respiratory apparatus. As explained above, the main controller of the respiratory apparatus is in electrical or data communication with the flow and/or pressure sensors located in the respiratory apparatus main housing, breathing conduit, and/or patient interface. In some example configurations, pressure sensing lines may feed pressure samples from a patient interface to a pressure sensor or sensors located in the main housing of the respiratory apparatus.

### 2.2 First Example WOB Indicator—ΔPₙₒₛₑ Estimation

A first example WOB indicator method or algorithm for generating a first example WOB indicator will now be described. The first example WOB indicator is based on determining or calculating a nasal pressure variation value, namely a ΔPₙₒₛₑ estimation, as will be explained in the following.

In general, the flow through a breathing conduit (Q_{tube}) can be determined by applying known fluid dynamics relationships. The result is the following equation:

$${Q}_{tube} = {C}_{tube} \sqrt{{P}_{blower} - {P}_{nose}} \qquad ( 1 )$$

### Where:

C_{tube} is the conductance of the breathing conduit-a measure of how easily/unimpeded gases can flow through the tube,
Q_{tube} is the flow rate of gases through the breathing conduit,
P_{blower} is the pressure at the output of the blower of the respiratory apparatus,
Pₙₒₛₑ is the pressure at or within a patient's nostrils, the ‘nasal pressure’.

In this example configuration, without access to direct nasal pressure measurements, some approximations and trial values may be used to find a tube conductance value C_{tube}. In other configurations, tube conductance can be derived or determined based on sensor data representing direct nasal pressure measurements.

In the context of NHF therapy delivered by a respiratory apparatus, it has been found that typically:

$${P}_{blower} \geq 10 {P}_{nose} \qquad ( 2 )$$

This expression allows for an approximation of tube conductance C_{tube} to be determined according to the below equation:

$${C}_{tube} = \frac{{Q}_{t u b e}}{\sqrt{{P}_{blower} - {P}_{nose , guess}}} \qquad ( 3 )$$

Where Pₙₒₛₑ,guess is a preliminary trial value for Pₙₒₛₑ. In this example, the P_{nose,guess} may be an initial estimate or guess of the nasal pressure. In one example, the initial estimate or guess may be based at least partly on the measured blower pressure and/or flow and/or a preliminary estimate for flow conductance. In one example, the initial estimate or guess may be a guess in a mathematical sense, i.e. not a completely arbitrary selection of a value, but rather an educated selection of an appropriate initial value that may be comparatively close to a true value. In an example configuration, the Pₙₒₛₑ,guess value may be derived or determined from one or more stored functions, prior relationships, equations, models or look-up tables, based on one or more input parameters such as, but not limited to, blower pressure, flow rate, and/or a preliminary estimate for flow conductance. In one example, the value of Pₙₒₛₑ, guess may be a function of the value of the blower output pressure P_{blower} based on prior knowledge of the expected or estimated pressure drop between the blower and patient interface (e.g. nasal cannula). In some configurations, most of the pressure drop is caused by the breathing conduit, for example.

In this example configuration, P_{blower} can be measured or sensed by one or more pressure sensors or pressure sensing configurations in the respiratory apparatus. In one example, the P_{blower} pressure data may be based on or a function of pressure data generated by a pressure sensor or sensors located at or near the outlet of the blower of the respiratory apparatus or in the flow path of the main housing of the respiratory apparatus downstream of the blower.

In one example configuration, the pressure sensor may be a gauge pressure sensor that includes a port to the ambient environment and which is configured to sense and generate pressure data representing the difference between the pressure at or near the blower outlet and the pressure in the ambient environment. In this configuration, the P_{blower} pressure data may comprise, be represented by, or be a function of the sensed gauge pressure data generated by the gauge pressure sensor.

In another example configuration, the pressure sensor may be an absolute pressure sensor that is configured to sense and generate pressure data representing the absolute pressure at or near the blower outlet in the flow path. In this configuration, the P_{blower} pressure data may comprise, be represented by, or be a function of the sensed absolute pressure data generated by the absolute pressure sensor.

In another example configuration, the apparatus may comprise an absolute pressure sensor that is configured to sense the absolute pressure at or near the blower outlet and an ambient pressure sensor that is configured to sense the ambient pressure of the environment. In this configuration, the P_{blower} pressure data may comprise pressure data representing the difference between the absolute pressure data from the absolute pressure sensor and ambient pressure data from the ambient pressure sensor.

In this example configuration, Q_{tube} may be based on the flow rate at the outlet of the blower of the respiratory apparatus, as sensed by a flow sensor of the apparatus. This assumes there are no faults or unintended behaviour such as a tube leak, e.g., due to an improper connection between the gases outlet of the main housing of the respiratory apparatus and the tube inlet. For example, assuming there are no significant leaks along the flow path from the blower to the patient interface connected at the end of the breathing conduit (e.g. tube), the flow rate Q_{tube} should be very close to the flow rate at the blower outlet.

As explained above, the tube flow rate Q_{tube} may be based on a flow rate signal or data from a flow rate sensor in the flow path of the main housing of the respiratory apparatus, downstream of the blower. In this example configuration, the Q_{tube} value is based on a pre-processed flow rate signal. For example, the raw flow rate signal or data from the flow rate sensor(s) may be processed to remove and/or minimise the effects of noise in the signal. Examples of pre-processing of the raw flow rate signal will be explained later in section 2.5.

In one example configuration, the selection or computation of Pₙₒₛₑ, guess (the preliminary trial values for Pₙₒₛₑ) may be pre-programmed and/or based on known relationships between the gas-flow parameters. In one example, the controller may be pre-programmed with a look-up table or other appropriate data structure or function that contains a range of approximate corresponding values for blower pressure P_{blower}, blower flow rate (equivalent to Q_{tube} as described above), and Pₙₒₛₑ values such that a trial Pₙₒₛₑ value (P_{nose,guess}) can be selected using or based on measures or sensor data representing the blower output pressure and flow. In one example configuration, the look-up table, function or data structure representing the relationship for selecting the trial Pₙₒₛₑ value may be stored in memory associated or accessible by the controller of the respiratory apparatus such as, but not limited to, non-volatile memory of the apparatus.

Once a trial nasal pressure Pₙₒₛₑ, guess value is chosen or selected, the breathing conduit conductance C_{tube} can be estimated according to the equation for C_{tube} provided above.

The C_{tube} equation can be rearranged into an equation to estimate nasal pressure Pₙₒₛₑ:

$${Q}_{tube}^{2} = {C}_{tube}^{2} ( {P}_{blower} - {P}_{nose} ) \qquad ( 4 )$$

$${P}_{nose} = {P}_{blower} - \frac{{Q}_{tube}^{2}}{{C}_{tube}^{2}} \qquad ( 5 )$$

As shown above, in this example, the estimate of nasal pressure Pₙₒₛₑ is determined or calculated as a function of the blower output pressure P_{blower}, tube flow rate Q_{tube}, and the tube conductance C_{tube}.

In this example, using an absolute estimate of nasal pressure Pose may be unreliable in some conditions, due to multiple sources of noise (e.g., blower motor noise, patient breathing, electronic noise, etc.) in the flow and pressure measurements that pre-processing steps may largely, but not entirely, account for. In view of this, the WOB indicator algorithm, in this configuration, may rely on a variable or parameter derived from the absolute value of the estimate of nasal pressure Pₙₒₛₑ to generate or represent the WOB indicator, rather than the absolute value itself.

In this example configuration, the WOB indicator may be based on the amplitude of fluctuations in the nasal pressure estimate (i.e., the ‘delta’), which is represented as ΔPₙₒₛₑ. This nasal pressure variation value ΔPₙₒₛₑ is correlated with a patient's breathing effort and can be observed as a WOB indicator as it is only weakly dependent upon the noise sources mentioned.

In this example configuration, the WOB indicator algorithm may be configured to determine an amplitude of the nasal pressure fluctuation (ΔPₙₒₛₑ) at a point in time using the following equation:

$$Δ {P}_{nose} \approx \frac{2 * {Q}_{tube} * M {V}_{device}}{{C}_{tube}^{2}} \qquad ( 6 )$$

Mathematically, this equation is essentially a derivative of the equation for Pₙₒₛₑ shown above. The variable MV_{device} term represents or is indicative of device minute ventilation (MV_{device}). In this example, MV_{device} estimates the average volume of air/gases mixture being output by the respiratory apparatus per minute. Although MV_{device} is related to the respiratory apparatus gases flow output, it is very closely related to the patient's breathing, as the patient's inhalation and exhalation volumes are encoded in the MV_{device} signal. Examples of methods for determining the MV_{device} signal or data indicative or representative of MV_{device} are described further below under section 2.6.

Because C_{tube} and Q_{tube} are approximately constant, changes in MV_{device} are reflected in the ΔPₙₒₛₑ value(s). Changes in MV_{device} appear due to changes in how hard a patient is breathing, i.e. how much energy is being expended when the patient breathes. In this example configuration, increasing ΔPₙₒₛₑ is indicative of increasing WOB, and conversely, decreasing ΔPₙₒₛₑ is indicative of decreasing WOB.

The nasal pressure variation signal or value ΔPₙₒₛₑ may be computed periodically or on an arbitrary or adhoc basis. In one example configuration, the nasal pressure variation signal or value ΔPₙₒₛₑ may be computed by the WOB indicator algorithm preiodically at any suitable frequency. In one example, the ΔPₙₒₛₑ value is computed as a WOB indicator at a frequency selected from the range of about 1 Hz to about 20 Hz (i.e., between about every 1 second to about every 5 ms). It will be appreciated that any suitable frequency may be selected depending on the applications of the WOB indicator and/or the characteristics of the incoming data streams used by the WOB indicator algorithm to calculated the ΔPₙₒₛₑ WOB indicator.

Any one or more of the following aspects relating to the ΔPₙₒₛₑ WOB indicator may apply in example configurations of the WOB indicator algorithm:
In some configurations, the ΔPₙₒₛₑ WOB indicator may have benefits compared to other WOB indicators disclosed due to comparatively fewer potential sources of error. For example, in one configuration, the ΔPₙₒₛₑ WOB indicator only requires or relies on flow rate and pressure sensor data or signals from the respiratory apparatus.
The tube conductance C_{tube} is approximately constant but in some example configurations may be updated at the same rate as ΔPₙₒₛₑ using a filter. This is because, in some scenarios or applications, the tube conductance may vary if/when the breathing conduit (e.g., tube) position changes during use of the respiratory apparatus to deliver respiratory therapy to a patient. For example, if the tube becomes more bent or curled, conductance will decrease (as impedance increases in these situations), or vice versa if the tube straightens during use.
In some example configurations, the flow rate of gases through the breathing conduit Q_{tube} should ideally be generally constant for maximum accuracy/reliability of the ΔPₙₒₛₑ WOB indicator generated. Over time periods of 1 minute or longer, this is generally the case. Nevertheless, in some configurations, the WOB indicator algorithm may be configured to account for variations/changes in the gases flow rate. For example, in some configurations, the WOB indicator algorithm may be configured to account for variations in the gases flow rate by discarding spurious readings associated with transient flow rate changes, and/or applying an averaging filter (e.g., an exponential filter) to smooth out the data.
In some example configurations, the value of the ΔPₙₒₛₑ WOB indicator may be dependent on size and fit of the patient interface used by the patient (e.g. the size and fit of the nasal cannula or other patient interface used by the patient). Additionally, or alternatively, the value of the ΔPₙₒₛₑ WOB indicator may be dependent on physiological characteristics of the patient such as, but not limited to, their breathing effort.
   2.3 Second Example WOB Indicator—ΔPₙₒₛₑ*Q_{breath} Estimation

A second example WOB indicator method or algorithm for generating a second example WOB indicator will now be described. The second example WOB indicator is based on or is a function of the nasal pressure variation value ΔPₙₒₛₑ of the first example WOB indicator and a variable representative or indicative of the change of breath volume or user's breath flow rate. In this example, the change of breath volume or user's breath flow rate is represented by Q_{breath}.

In one example configuration, the second WOB indicator algorithm may be a variation of the first WOB indicator algorithm. For example, the second WOB indicator algorithm may comprise additional steps or calculations beyond calculating the ΔPₙₒₛₑ WOB indicator, as will be explained further below.

In systems where volume changes, work performed can be expressed as:

$$Work = Pressure ⋆ Volume \qquad ( 7 )$$

In this second example, if the nasal pressure variation value ΔPₙₒₛₑ is substituted for Pressure, then an actual Work value for WOB may be estimated by substituting a measure or value of the change in volume for the Volume term. In this example, the result of these substitutions provides an estimate of an actual Work value for WOB, rather than an indicator of WOB. By way of example, the estimate of actual Work provides a value with units in joules.

In this second example WOB indicator algorithm, a measure or value representing a change of breath volume or the user's breath flow rate is represented as Q_{breath}. In this example, the user's breath flow rate Q_{breath} may be approximated via an equation:

$${Q}_{breath} = \sqrt{{Q}_{tube}^{2} + ( \frac{{C}_{nose}^{2}}{{C}_{tube}^{2}} ) ( 2 {Q}_{tube} {MV}_{device} + {MV}_{device}^{2} )} - {Q}_{tube} + M {V}_{device} \qquad ( 8 )$$

Where all the terms have the same physical meaning as previously described. Cₙₒₛₑ represents the conductance of the patient nostrils or nares, and may be a function of at least the size of the patient's nostrils and the nasal cannula size and fit. While C_{tube} can be readily approximated (as previously discussed), the parameters that Cₙₒₛₑ is dependent upon may require implementation of a calibration or learning process to obtain an accurate estimate, in this example configuration.

In this example configuration, the user's breath flow rate Q_{breath} may be physically similar to the nasal minute ventilation of a patient, which is represented by MVₙₐₛₐₗ. Nasal minute ventilation MVₙₐₛₐₗ is a metric for estimating the minute ventilation of a patient's nasal cavity, and is explained in detail in PCT Patent Application Publication WO2022/167960, filed 3 Feb. 2022, which is incorporated by reference herein in its entirety. Unlike the MVₙₐₛₐₗ metric however, the user's breath flow rate Q breath may better account for the flow conductance components in a high flow therapy system (e.g. a respiratory apparatus that is configured to deliver NHF therapy) and therefore may be a more versatile indicator of the average quantity of air entering/exiting a patient's nasal cavity per minute. In this example configuration, MVₙₐₛₐₗ may be similar to MV_{device} except transformed by a factor that incorporates information and/or is a function of the patient interface (e.g. size, type etc) and the fit of the patient interface (e.g. degree of occlusion of the nares in the context of a nasal cannula).

In this example configuration, the user's breath flow rate Q breath equation or function is derived by applying a flow-pressure equation to a suitable patient nostril model: Conductance*Pressure=Flow². Alternatively, Pressure=Resistance*Flow² may be used. In one example, the patient nostril model is based at least partly on three key flows, namely: flow in and flow out of nostrils via the nasopharynx due to patient breathing, and leakage flow out of/around the nasal cannula. This results in three simultaneous equations (9)-(11) which can be rearranged and solved for the patient's breath flow. In this example, the resulting expression for Q_{breath} can then be integrated (e.g., to average it). Thus, the equation above (for determining the average value of Q_{breath}) is arrived at.

By way of example, the three simultaneous equations may be:

$${C}_{tube} * ( {P}_{device} - {P}_{nose} ) = {Q}_{tube}^{2} \qquad ( 9 )$$

$${C}_{nose} * {P}_{nose} = {Q}_{nose}^{2} \qquad ( 10 )$$

$${Q}_{breath} + {Q}_{tube} = {Q}_{nose} \qquad ( 11 )$$

The above three simultaneous equations (9)-(11) are some examples of possible equations that may be used and solved for the patient's breath flow. In alternative configurations, one or more different and/or more complex equations that include more parameters may be used if more accurate modelling is desired or required for a particular application or circumstance.

In one example configuration, a calibration process to obtain a Cₙₒₛₑ estimate or value may entail inserting a suitable nasal cannula into the patient's nares, followed by the respiratory apparatus trialling a range of pre-determined flow rates, concurrently measuring or estimating nasal pressure at each discrete flow rate. Additionally or alternatively, in another example configuration, the calibration process may be non-discrete, involving provision of a sweep of flow rates while nasal pressure is continuously measured or estimated.

In another alternative example configuration, using manual inputs from a user or clinician, a value for Cₙₒₛₑ may be estimated based at least partly on one or more patient physiological factors or characteristics. For example, a value for Cₙₒₛₑ may be estimated based on parameters or estimates relating to patient size (e.g., their body length) and/or cannula-nostril occlusion (e.g., a percentage occlusion of the nares by cannula prongs estimate), and/or one or more other suitable parameters.

By way of further explanation, in regard to patient size, a longer (larger) patient may be expected to have larger nares (e.g., adult versus paediatric or even infant patients). The size (e.g., cross-sectional area of the entrance of the nares) will contribute to Cₙₒₛₑ. Ignoring whether or not the nasal cannula is fitted optimally, larger patients will generally don a larger cannula than smaller patients such as paediatrics, and larger cannula will have wider-bore prongs that provide less flow resistance.

By way of further explanation, in regard to cannula-nostril occlusion (or ‘naris occlusion’), a higher percentage occlusion means a closer fit between the inside of the nares and the walls of the cannula prongs, and conversely, a lower percentage occlusion means there is more space between the cannula prongs and the inside of the naris. Depending on the degree of occlusion, the amount/rate of gases flow exhaled via the prongs or the prong-naris ‘gap’ will vary and thus affect the flow conductance.

In this example, the Cₙₒₛₑ estimation may be based on relationships determined from prior empirical testing.

This second example WOB indicator algorithm generates a WOB indicator that represents an estimation of ΔPₙₒₛₑ*Q_{breath}, which may be considered an actual estimate of WOB, as described above. In this example configuration, this ΔPₙₒₛₑ*Q_{breath} WOB indicator may involve more computational steps than the first example ΔPₙₒₛₑ WOB indicator. In this example, the additional computational steps may primarily arise from the need to estimate or derive a value for the conductance of the patient's nostrils Chose. This second example ΔPₙₒₛₑ*Q_{breath} WOB indicator may provide an actual estimate of WOB that may be more familiar to clinicians. In this example, the accuracy and reliability of the second example ΔPₙₒₛₑ*Q_{breath} WOB indicator may be at least partly dependent on the accuracy of the Cₙₒₛₑ value. In some configurations, the accuracy of the Cₙₒₛₑ value may be at least partly reliant or dependent on additional input or parameters provided by a clinician regarding the patient, as described above.

### 2.4 Third Example WOB Indicator—ΔPₙₒₛₑ*Breath Smoothness Estimation

A third example WOB indicator method or algorithm for generating a third example WOB indicator will now be described. The third example WOB indicator is based on or a function of the nasal pressure variation value ΔPₙₒₛₑ of the first example WOB indicator and a variable representative or indicative of a breath smoothness factor. In this example, the breath smoothness factor is represented by σ.

In one example configuration, the third WOB indicator algorithm may be a variation of the first WOB indicator algorithm. For example, the third WOB indicator algorithm may comprise additional steps or calculations beyond calculating the ΔPₙₒₛₑ WOB indicator, as will be explained further below.

In this third example, the WOB indicator algorithm is configured to apply a breath smoothness factor σ to the ΔPₙₒₛₑ estimate to thereby generate a ΔPₙₒₛₑ*σ WOB indicator. In this example, breath smoothness is based on a mathematical concept which quantifies the number of possible continuous derivatives of a function over a domain.

In the case of the ΔPₙₒₛₑ data, the breath smoothness physically relates to the rate of change of the nasal pressure fluctuations.

This breath smoothness measure σ is useful as it can be indicative of (or strongly correlated with) the respiratory rate of a patient. By way of example, higher respiratory rates will be apparent in the nasal pressure fluctuation signal or data as sharper rates of change, which will be measured by the breath smoothness value σ (i.e., a value representing reduced breath smoothness), and vice versa (i.e., increased breath smoothness values may represent lower respiratory rates).

In one example configuration, the third WOB indicator algorithm may determine or calculate a breath smoothness factor σ based at least partly on a patient minute ventilation (e.g., device minute ventilation) estimate or value. By way of example, the WOB indicator algorithm may be configured to estimate device minute ventilation according to any one of the methods outlined below in section 2.6 or any other suitable methods. In this example, the calculated device minute ventilation estimates may then be normalised. By way of example, the device minute ventilation may be normalised according to or based on the number of data points available and then passed to a function that outputs a frequency-independent breath smoothness measure or factor σ. In this example, the function may be pre-determined through a mixture of analytic and numerical analysis.

The third WOB indicator algorithm is then configured to apply the calculated breath smoothness factor σ to the ΔPₙₒₛₑ WOB indicator (e.g., of the first example WOB indicator) to generate the third example WOB indicator, ΔPₙₒₛₑ*σ. In this example, the ΔPₙₒₛₑ*σ WOB indicator may be mathematically more dependent on respiratory rate compared to the first example ΔPₙₒₛₑ WOB indicator. In this configuration, the third example WOB indicator may be more similar to the well-established pressure-time product metric, which is another known WOB metric.

### 2.5 Pre-Processing of Flow Rate Signal

As explained above, the example WOB indicator methods or algorithms and their respective output WOB indicators are at least partly based on flow parameter data. In the examples, the flow parameter data may at least include flow rate data from one or more flow rate sensors that is representative or indicative of the flow rate of gases in the flow path. As further explained, typically the WOB indicator algorithms receive and utilise a pre-processed flow rate signal or data, i.e., the raw flow rate data or signal from the flow sensor(s) is pre-processed to remove or minimise the effects of noise in the signal. In alternative configurations, it will be appreciated that the WOB indicator algorithms may receive the raw flow rate signal or data and perform the pre-processing steps or stages as part of the WOB indicator algorithm.

Examples of some forms of pre-processing that may be applied to the raw flow rate signal or data before it is further processed and/or used as an input for generating a WOB indicator will now be described below.

As discussed above, the flow data (e.g., flow rate data) in an unsealed system, such as nasal high flow systems, can be difficult to determine. The open nature of the system results in a very low signal to noise ratio. For example, the unsealed interface (nasal cannula) tends to result in a lot of leakage and swirling flows around the patient's nares, which contributes a substantial amount of noise in the sensed flow rate signal (e.g., raw flow rate data). Any flow data measured can include various irregularities and noise that can obscure the flow data and which must be accounted for to accurately determine the desired measurement. Flow data is important as it can be informative of the unsealed system, patient breath flow, and/or other apparatus or patient metrics and/or parameters.

In this example, in order to remove noise and other irregularities from any obtained flow data, the flow signal can be fed through pre-processing steps or stages. Pre-processing can allow the controller to remove certain distortions from the flow parameters, such that the flow parameter signal that is used to determine the apparatus output and/or patient breathing parameters (e.g., WOB indicators) can better reflect the effect the gas flow parameter used in the patient's treatment is having on the patient's respiration. More details of pre-processing of flow signals are described in PCT Application Publication WO/2020/178746, filed 4 Mar. 2020, which is incorporated by reference herein in its entirety.

If the patient is attached to the respiratory system and breathing through the patient interface, fluctuations in pre-processed flow rate or other flow parameter data obtained in an open system (i.e. unsealed system that delivers the flow of gases to the patient via an unsealed interface such as a nasal cannula) are made up of random uncorrelated noise and a correlated breathing signal generated from various sources. In particular, fluctuations in the flow parameter data can comprise noise (random and uncorrelated with the patient's breathing) and the patient breathing signal (which is correlated with the patient's breathing). The pre-processing of the data can start with the controller receiving the flow parameter data (such as unprocessed or raw data). The controller can then perform the pre-processing step, for example, by determining if the flow parameter data is good or suitable for use. If the data is not suitable for use, the controller can discard the data.

In determining the suitability of the data, the controller can receive second flow parameter data that is of a different type than a first flow parameter data. The second flow parameter data is assumed to have some correlation to the first parameter. The second flow parameter data can include, for example, the motor speed, pressure, and/or oxygen flow rate or concentration or any other parameter that can have an effect on or provide an indication of the gases flow rate that is separate from the effect of the patient's respiration on the gases flow rate. The controller can be configured to determine whether the second flow parameter data is useful as a correlation parameter to the first flow parameter data. For example, the second flow parameter data can be a useful correlation metric if the second flow parameter data meets a threshold level. If the second flow parameter data does not meet a threshold level, then it is assumed the second flow parameter data is not correlated to the first flow parameter data. As such, the second flow parameter data can be ignored or thrown out (i.e., deleted or not utilized by the controller). If there is insufficient second flow parameter data, the controller can determine that it has insufficient data to use the first flow parameter data and may discard the first parameter data. If the second flow parameter data meets a minimum threshold level, the controller can determine that the first parameter data is suitable for use.

As an example, the second flow parameter data can represent the motor speed. In order to identify the patient's respiration in the first flow parameter data, the motor needs to be operating at a sufficient speed. If the motor speed is too low, the effect or correlation of the motor speed on the flow data (such as the flow rate) may not be accurately predictable. Therefore, after the controller has received the motor speed data, the controller can compare the motor speed to a minimum motor speed threshold. If the motor speed is below the threshold, the controller can deem the first flow parameter data as unsuitable and can discard a portion or all of the first flow parameter data. However, if the motor speed is above the threshold, the controller can calculate the recent changes in the motor speed. A change in motor speed can result in a change in the first flow parameter data, which makes it more difficult to identify the patient's respiration in the first flow parameter data. While the effect of the motor speed can be removed from the first flow parameter data to some degree, larger changes in motor speed may make the data too unreliable for identifying the patient's respiration. Therefore, the controller can apply a running filter to the relative changes in motor speed in order to generate a first value representing the recent relative changes in motor speed. The controller can then compare the first value with a first threshold. If the first value is above the first threshold, the controller can deem the flow parameter data to be unsuitable, and the flow data point can be discarded. If the first value is below the first threshold, the controller can deem the flow parameter data to be suitable for use.

As another example, the second flow parameter data can represent the concentration of a supplementary gas from a supplementary gas source. The first flow parameter data (such as the flow rate) can be affected by the flow rate or concentration of supplementary gas from a supplementary gas source. The controller can receive an oxygen flow rate data or an oxygen concentration data. The controller can calculate recent changes in the oxygen flow rate or the oxygen concentration. If the flow rate or concentration of oxygen changes, the resulting change in the total flow rate can make it more difficult to identify the patient's respiration in the flow rate signal or other flow parameter signal. The controller can therefore apply a running filter to the changes in oxygen concentration of the gases or the oxygen flow rate in order to generate a second value representing the recent changes in oxygen concentration or flow rate. The controller can compare the second value with a second threshold. If the second value is above the second threshold, the controller can determine the first flow parameter data is unsuitable, and the first flow parameter data point can be discarded. However, if the second flow parameter data is below the threshold, the controller can deem the flow parameter data to be suitable.

As described above, if the controller deems the data to be suitable, the first flow parameter data (or any other flow parameter data) can also be modified to remove the effect of the motor (or other factors, such as the oxygen concentration or flow rate). Modifying the first flow parameter data can involve removing the assumed effect of other variables from the first flow parameter data (such as the motor speed). This assumed effect is only valid if the gases flow parameter data meets certain criteria. As described above, if these criteria are not met, the data may be discarded.

The process can modify the first flow parameter data to remove the effect of motor speed. The effect of the motor can be estimated using the motor speed and the flow conductance.

The controller can measure an instantaneous flow conductance. The flow conductance can be calculated as provided below:

$$C = filt ( \frac{Q}{{ω}_{motor}} )$$

In the equation provided above, C is the flow conductance, filt( ) is a filter function, Q is the flow parameter data, and @motor is the motor speed. In some configurations, the filter function is a low-pass filter. In some examples, the flow parameter data is a flow rate signal generated by a flow rate sensor of the respiratory apparatus. The flow conductance is approximately constant with time, and can therefore be estimated using a low pass filter. The controller measures the instantaneous flow conductance at each iteration using the current motor speed and a measured flow rate. The controller can filter the instantaneous flow conductance in order to determine a filtered flow conductance.

The controller can compare the instantaneous flow conductance with the filtered flow conductance to see if the difference is significantly different. If the difference is significant, it is likely that something has changed the physical system, such as the cannula being attached or detached. The instantaneous flow conductance can be compared with the filtered flow conductance by taking the difference of the two variables and comparing it with a minimum or a maximum threshold. If the difference exceeds or falls below the threshold, the difference is considered to be significant, and the controller can reset the filtered flow conductance. The controller can also vary the filter coefficient of the filter function in the filtered flow conductance calculation based on the difference between the instantaneous flow conductance and the filtered flow conductance. This allows the filtered flow conductance to change more quickly when the variance of the flow conductance is high, such as when the cannula has first been attached.

If the difference between the instantaneous flow conductance with the filtered flow conductance does not exceed the threshold, the difference is considered to be not significant, and the controller can estimate the effect of the motor on the flow rate. The controller can output a value of the effect using the filtered flow conductance and the motor speed. The value can be subtracted or otherwise removed from the flow rate data to arrive at the pre-processed flow rate data. The pre-processed flow rate data can be more indicative of the patient's respiratory flow (although the pre-processed flow rate data can still include signal noise).

The controller can also track the recent changes in the flow conductance. The changes can be tracked by adding the difference between the last two instantaneous flow conductance values to a running total, which is then decayed over time. The decayed running total is filtered to obtain the filtered recent changes in flow conductivity. The filtered recent changes in flow conductivity can be used in further parts of the frequency analysis algorithm along with the pre-processed flow rate data.

### 2.6 Determining Device Minute Ventilation MV_{device}

As explained above, some of the example WOB indicator algorithms and their respective WOB indicators are at least partly based on or a function of the device minute ventilation MV_{device} of the respiratory apparatus.

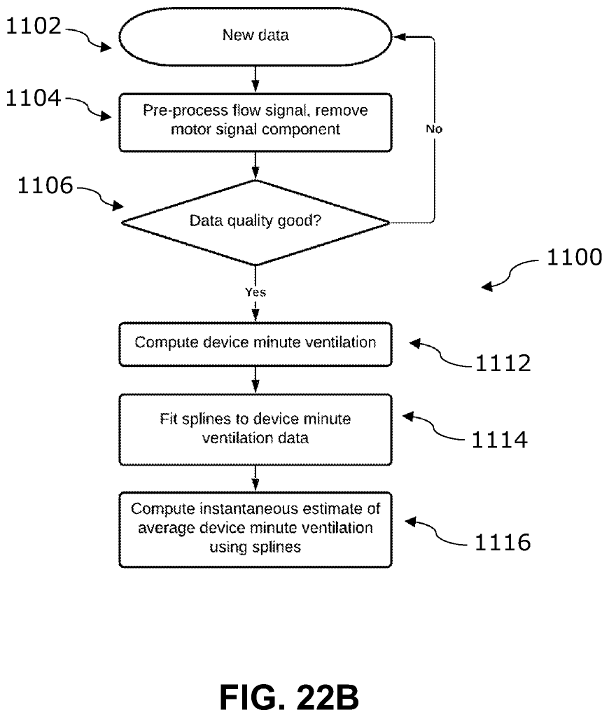

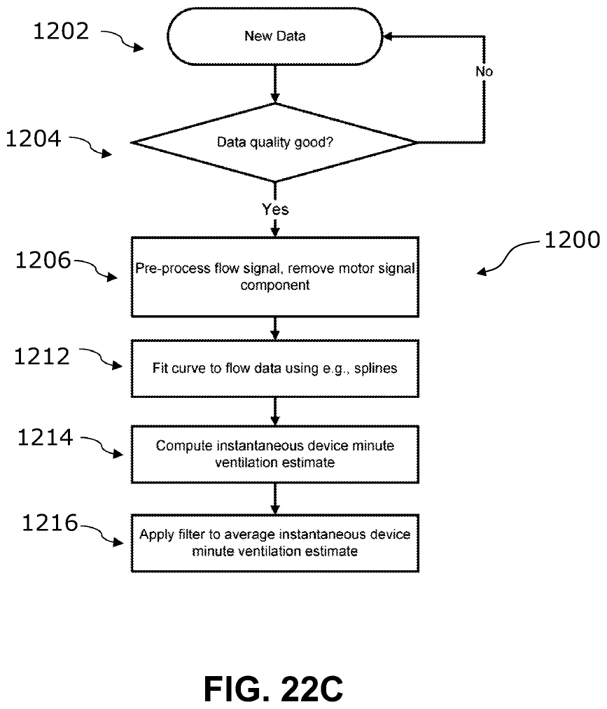

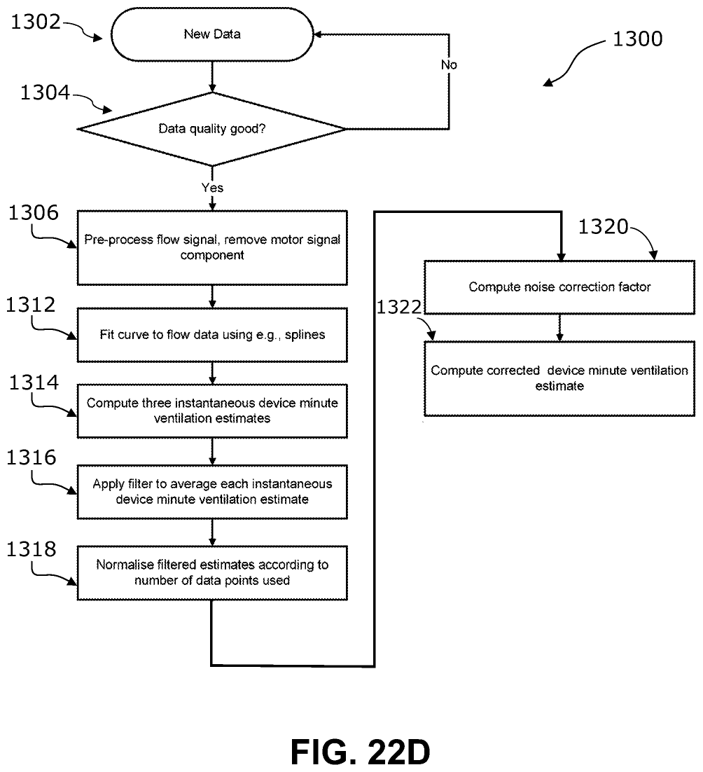

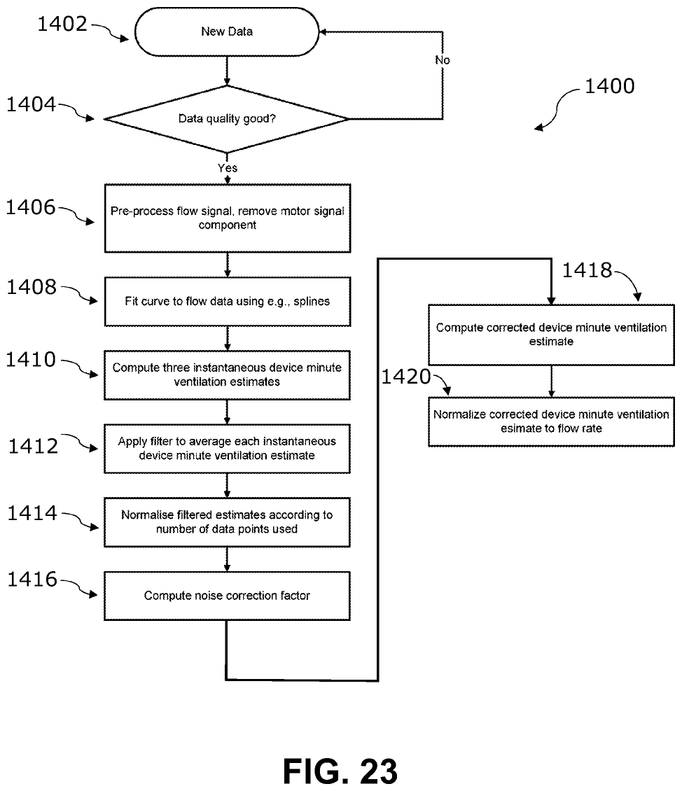

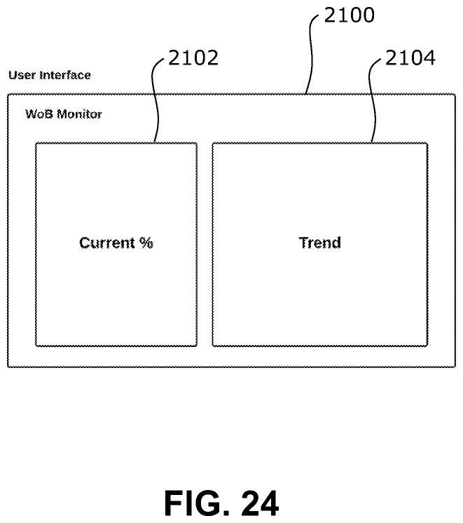

With reference to FIGS. 22A-23, examples of various methods for determining or calculating estimates of device minute ventilation MV_{device} will be described below, although it will be appreciated that alternative methods may be used. In some configurations, the WOB indicator algorithms may implement such methods for determining device minute ventilation estimates or data. In other configurations, device minute ventilation estimates or data may be computed separately in the controller and then fed to the WOB indicator algorithms executing on the controller.

While inhalation and exhalation are relatively easy to measure in sealed systems, breathing parameters are much more difficult to measure in unsealed systems. In unsealed systems, such as a nasal high flow system, the open nature of the system (due to use of an unsealed patient interface) makes it significantly more difficult to determine patient breathing parameters because the desired signal(s) are often weak and/or obscured by noise.

The present disclosure provides reliable methods of estimating key patient breathing parameters (e.g. WOB indicators) in an unsealed system (e.g., unsealed nasal cannula used in a high flow system).

The controller can include processes that are configured to calculate an estimate of device minute ventilation (MV_{device}). The device minute ventilation (MV_{device}) represents the device minute ventilation, a measure of the average volume of air being pushed in and out of the respiratory apparatus (i.e. device) per minute. In some configurations, the device minute ventilation (MV_{device}) may be discrete values or a series of discrete values (e.g., a sequence of prior estimates). In some examples, the series of discrete values may begin from the first estimation of the device minute ventilation (MV_{device}) and continue until the end of a respiratory apparatus use (therapy) session. Alternatively, a series of discrete values of the device minute ventilation (MV_{device}) estimates may represent a specific window of time and be continuously overwritten as new values are estimated.

For example, a discrete series of values of the device minute ventilation (MV_{device}) estimates may represent their values over a recent period of time. In some configurations, the period of time can be 15 minutes, 20 minutes, 25 minutes, 30 minutes, 35 minutes, 40 minutes, 45 minutes, 50 minutes, 55 minutes, 60 minutes, 65 minutes, 70 minutes, between 15-20 minutes, between 20-25 minutes, between 25-30 minutes, between 30-35 minutes, between 35-40 minutes, between 40-45 minutes, between 45-50 minutes, between 50-55 minutes, between 55-60 minutes, between 60-65 minutes, between 65-70 minutes, between 15-30 minutes, between 30-45 minutes, between 45-60 minutes, and any values in between those ranges listed, including endpoints.

FIGS. 22A, 22B, 22C, and 22D illustrate four flow charts for a method of estimating device minute ventilation (MV_{device}). FIG. 22A illustrates a simplified method 1000 of estimating device minute ventilation (MV_{device}) 1000. FIGS. 22B, 22C, and 22D illustrate examples of more detailed methods 1100, 1200, 1300 of estimating device minute ventilation (MV_{device}).

As shown in FIG. 22A, a method 1000 of estimating device minute ventilation (MV_{device}) starts by obtaining raw flow rate data at step 1002. The raw flow rate data can be acquired from a flow rate sensor such as an ultrasonic flow sensor. The method of estimating device minute ventilation (MV_{device}) can include pre-processing the raw flow rate data to remove unwanted signal components at step 1004. Removal of unwanted signal components has been described in more detail above. The unwanted signal components can be present from the flow generator motor. In some configurations, unwanted signal components can be generated from other sources (e.g., noise), the unwanted signal components can be primarily from the flow generator motor. For example, the pre-processed flow data can be a flow rate signal comprising components associated with a patient's breathing activity. For example, components associated with the patient's breathing activity may be included in the flow rate signal as changes in the flow rate magnitude (e.g., as fluctuations). Once pre-processed, the flow data can be representative of patient breathing data. The method 1000 can include step 1006 where, assuming the data is of sufficient quality, the pre-processed data can be passed to a device minute ventilation algorithm to calculate the device minute ventilation (MV_{device}).

Referring to FIG. 22B, a more detailed flowchart of a method 1100 of estimating device minute ventilation (MV_{device}) will be described. As with the method 1000, the method 1100 starts by obtaining raw flow rate data at step 1102. The raw rate flow data can be acquired from a flow rate sensor such as an ultrasonic flow sensor. The method 1100 can include pre-processing the raw flow rate data to remove unwanted signal components at step 1104. Removal of unwanted signal components is described in more detail above. The unwanted signal components can be present from the flow generator motor. Once pre-processed, the flow data can be representative of patient breathing data. The processed flow rate data can then analyzed at step 1106 to determine whether the data quality is sufficiently good. If not, the flow data is discarded and the method 1100 returns to step 1102 and waits to receive raw flow data.

However, if the data is determined to be of sufficient quality, the data can progress to step 1112 wherein the method 1100 computes the device minute ventilation (MV_{device}) using the processed data. The data can be considered of sufficient quality if it does not include large transient peaks (perhaps due to interface adjustment). The device minute ventilation (MV_{device}) measures the average volume of air being pushed in and out of the device per minute. As shown in step 1112, the process for computing the device minute ventilation (MV_{device}) can be done by first fitting splines to the flow data using the least squares criterion. The flow data may be, for example, the most recent pre-filtered flow rate data points. In some configurations, a least squares criterion first approximates the pre-processed flow data (e.g., the breathing signal) and then integrates along the splines to estimate ventilation volumes.

The method 1100 can include step 1116 wherein an instantaneous estimate of device minute ventilation (MV_{device}) is computed using splines. In some examples, the use of splines can be useful over alternative methods (e.g., a series of filters configured to generate statistical measures of the data) because it can perform better (i.e., more accurately fit/interpolate the data) across a wider range of sampling frequencies. This is described in more detail below. The method 1100 can include three different methods of computing instantaneous estimates of the device minute ventilation (MV_{device}). An estimate of the device minute ventilation (MV_{device}) represents the integral of the absolute value of the first term of the line fitted to the data (û_{fit}) of the parameter of the flow of gases, divided by the time range covered by the selected filtered flow rate data points (i.e., a zero-order spline). An estimate of the device minute ventilation (MV_{device}) is represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds.

An estimate of the device minute ventilation (MV_{device}) represents the average of the absolute value of the curve fitted to the data of the parameter of the flow of gases (i.e., computed without using splines for data interpolation). Any of the aforementioned estimates can be used in the method 1100 as the input, although each have advantages and disadvantages depending on the random error in the sensor data and the patient respiration rate. The method 1100 can use an estimate of the device minute ventilation (MV_{device}) that represents the integral of the absolute value of the first term of the line fitted to the signal. This estimate can be the most resilient to and least influenced by random error while also being the most influenced by respiration rate.

Referring to FIG. 22C, another more detailed flowchart of a method 1200 of estimating device minute ventilation (MV_{device}) will be described. As with the method 1000 and method 1100, the method 1200 starts by obtaining raw flow rate data at step 1202. The raw flow rate data can be acquired from a flow rate sensor such as an ultrasonic flow sensor. In some configurations, at step 1204, the raw flow rate data is first analyzed to determine whether the data quality is sufficiently good. If the raw flow rate data is determined to be of sufficient quality, the data can be pre-processed to remove unwanted signal components. If the raw flow rate data is of insufficient quality, the flow rate data is discarded and the method 1200 returns to step 1202 and waits to receive additional raw flow data. In some examples, the method 1200 can include pre-processing the raw flow rate data to remove unwanted signal components at step 1206. Removal of unwanted signal components is described in more detail above. The unwanted signal components can be present from the flow generator motor. Once pre-processed, the flow data can be representative of patient breathing data. In some configurations, once the flow data is pre-processed, the data can progress to step 1212 wherein the method 1200 fits a curve to the flow data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + s {t}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1200 can include step 1214 wherein an instantaneous estimate of device minute ventilation (MV_{device}) is computed using the data of the curve constructed by the fitted splines. The method 1200 can include three different methods of computing instantaneous estimates of the device minute ventilation (MV_{device}). An estimate of the device minute ventilation (MV_{device}) is represented by the integral of the absolute value of the first term of the line fitted to the data (û_{fit}) of the parameter of the flow of gases, divided by the time range covered by the selected filtered flow rate data points (i.e., a zero-order spline). An estimate of the device minute ventilation (MV_{device}) is represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds.

An estimate of the device minute ventilation (MV_{device}) represents the average of the absolute value of the curve fitted to the data of the parameter of the flow of gases (i.e., computed without using splines for data interpolation). Any of the aforementioned estimates can be used in the method 1200 as the input, although each have advantages and disadvantages depending on the random error in the sensor data and the patient respiration rate. The method 1200 can use an estimate of the device minute ventilation (MV_{device}) that represents the integral of the absolute value of the first term of the line fitted to the signal. This estimate can be the most resilient to and least influenced by random error while also being the most influenced by respiration rate. In some implementations, the method 1200 can skip step 1212 and the pre-processed flow data can directly proceed to step 1214 wherein the method 1200 can conduct a direct computation of the average of the selected pre-processed flow rate data points.

In one example configuration, the method 1200 may also be configured to apply a filter to average the instantaneous device minute ventilation (MV_{device}) at step 1216. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps.

In some configurations, an estimate of device minute ventilation (MV_{device}) is determined by taking an estimate that involves taking the integral of the absolute value of the first term of the line fitted to the flow rate signal (e.g., zero-order spline). In some examples, the device minute ventilation (MV_{device}) is estimated for each of the data points using all three approaches discussed above. As previously mentioned, the three methods include: (1) where an estimate of the device minute ventilation (MV_{device}) is represented by the integral of the absolute value of the first term of the line fitted to the data (û_{fit}) of the parameter of the flow of gases, divided by the time range covered by the selected filtered flow rate data points (i.e., a zero-order spline); (2) where an estimate of the device minute ventilation (MV_{device}) is represented by the integral of the absolute value of the line fitted to the data (û_{fit}) of the parameter of the flow of gases, divided by the time range covered by the selected filtered flow rate data points (i.e., a first-order spline); and (3) where an estimate of the device minute ventilation (MV_{device}) represents the average of the absolute value of the curve fitted to the data of the parameter of the flow of gases (i.e., computed without using splines for data interpolation).

Referring to FIG. 22D, another flowchart of a method 1300 of estimating device minute ventilation (MV_{device}) 1300 will be described. In this method 1300, the device minute ventilation (MV_{device}) is estimated for each of the data points using all three approaches discussed above. As with the methods 1000, 1100, 1200, the method 1300 starts by obtaining raw flow rate data at step 1302. The raw flow rate data can be acquired from a flow rate sensor such as an ultrasonic flow sensor. In some configurations, at step 1304, the raw flow rate data is first analyzed to determine whether the data quality is sufficiently good. If the raw flow data is determined to be of sufficient quality, the data can be pre-processed to remove unwanted signal components. If the raw flow data is of insufficient quality, the flow data is discarded and the method 1300 returns to step 1302 and waits to receive additional raw flow rate data. In some examples, the method 1300 can include pre-processing the raw flow data to remove unwanted signal components at step 1306.

Removal of unwanted signal components is described in more detail above. The unwanted signal components can be present from the flow generator motor. Once pre-processed, the flow rate data can be representative of patient breathing data. In some configurations, once the flow rate data is pre-processed, the data can progress to step 1312 wherein the method 1300 fits a curve to the flow rate data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + s {t}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1300 can include step 1314 wherein three instantaneous estimates of device minute ventilation (MV_{device}) are computed using the data of the curve constructed by the fitted splines. The method 1300 can include three different methods of computing instantaneous estimates of the device minute ventilation (MV_{device}). In some configurations, one of the three estimates is an estimate of the device minute ventilation (MV_{device}) represented by the integral of the absolute value of the first term of the line fitted to the data (û_{fit}) of the parameter of the flow of gases, divided by the time range covered by the selected filtered flow rate data points (i.e., a zero-order spline). In some examples, another of the three estimates is an estimate of the device minute ventilation (MV_{device}) represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds. In some configurations, another of the three estimates is an estimate of the device minute ventilation (MV_{device}) represented by the average of the absolute value of the curve fitted to the data of the parameter of the flow of gases (i.e., computed without using splines for data interpolation). In some configurations, the first MV_{device} estimate (i.e., zero-order spline) is most resilient to noise/least dependent upon it, and is most influenced by patient respiratory rate. In some examples, the second MV_{device} estimate (i.e., first-order spline) is very noise-dependent but is less dependent of respiratory rate. In some configurations, the third MV_{device} estimate (i.e., wherein there are no splines/interpolation and the estimate is a direct averaging of a series of instantaneous absolute values) is highly affected by noise and is independent of respiratory rate. It is further noted that all three estimates are flow-variant, meaning they will vary according to the blower flow output.

In some configurations, the method 1300 can skip step 1312 and the pre-processed flow data can directly proceed to step 1314 wherein the method 1300 can conduct a direct computation of the average of the selected pre-processed flow rate data points.

Once the method 1300 obtains the three estimates of the device minute ventilation (MV_{device}) at step 1314, a filter can be applied to the device minute ventilation (MV_{device}) to average the instantaneous device minute ventilation (MV_{device}) at step 1316. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps.

In the method 1300, there are three measurements (i.e., the first MV_{device} estimate, the second MV_{device} estimate, and the third MV_{device} estimate) and three unknown values or signal components that can constitute and/or contribute to the device minute ventilation signal estimated. These unknown values and signal components can include, for example, noise, respiratory rate (e.g., the speed of flow change induced by the patient), and the underlying device minute ventilation signal. Therefore, there exists a set of three analytic expressions or equations that may be solved simultaneously to derive expressions for noise, respiratory rate, and device minute ventilation. In some configurations, simultaneously solving the three analytic expressions or equations can be highly computationally expensive and therefore demanding on processing hardware in embedded device applications, such as in medical devices. In some configurations, in the method 1300, the algorithm of the method 1300 first proceeds to step 1318 to normalize the three MV_{device} estimates according to the number of data points used in each of the estimations. In some examples, the method 1300 can include step 1320 wherein a noise correction factor can be computed, wherein the noise correction factor has a relationship with the signal-noise ratio. The calculation of a noise correction factor in step 1320 can be similar to any of those disclosed in PCT Application Publication WO/2020/178746, filed 4 Mar. 2020, which is incorporated by reference in its entirety. In some configurations, a noise correction factor that is related to one or more of the normalized MV_{device} estimates may be computed.

In some configurations, the method 1300 can include step 1322 wherein the algorithm can use a pre-defined fitted curve that relates the normalized minute ventilation estimates and the noise correction factor to one of the device minute ventilation (MV_{device}) estimates. In some configurations, the fitting function may comprise, at least partially, some numerically-derived terms. In some configurations, this corrected curve can approximate the output of an analytic expression for the device minute ventilation (MV_{device}). This can provide a device minute ventilation (MV_{device}) that has a minimal noise and respiratory rate dependency.

In some configurations of the method 1300, each device minute ventilation (MV_{device}) estimate or sequence of estimates captured over multiple repetitions of the previously described steps can be averaged or “smoothed” using a filter (e.g., an exponential filter). In some examples, this step can occur after the initial estimation but prior to any additional processing steps.

Referring to FIG. 23, a flowchart of a method 1400 of estimating a normalized device minute ventilation will be described. Unlike the methods illustrated in FIGS. 22B-22D, the respiratory apparatus takes the corrected device minute ventilation and normalizes it using the respiratory apparatus flow rate. In some configurations, this can render an estimate of device minute ventilation that is flow-invariant and independent of the device flow rate. In some examples, the estimated device minute ventilation is also independent of nasal cannula fit and can provide a versatile indicator of patient minute ventilation.

The method 1400 starts by obtaining raw flow rate data at step 1402. The raw flow rate data can be acquired from a flow rate sensor such as an ultrasonic flow sensor. In some configurations, at step 1404, the raw flow rate data is first analyzed to determine whether the data quality is sufficiently good. If the raw flow rate data is determined to be of sufficient quality, the data can be pre-processed to remove unwanted signal components. If the raw flow rate data is of insufficient quality, the flow rate data is discarded and the method 1400 returns to step 1402 and waits to receive additional raw flow rate data. In some examples, the method 1400 can include pre-processing the raw flow rate data to remove unwanted signal components at step 1406. Removal of unwanted signal components is described in more detail above. The unwanted signal components can be present from the flow generator motor.

In some configurations, once the flow data is pre-processed, the data can progress to step 1408 wherein the method 1400 fits a curve to the flow data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + s {t}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1400 can include step 1410 wherein three instantaneous estimates of device minute ventilation (MV_{device}) are computed using the data of the curve constructed by the fitted splines. The method 1400 can include three different methods of computing instantaneous estimates of the device minute ventilation (MV_{device}). In some configurations, one of the three estimates is an estimate of the device minute ventilation (MV_{device}) represented by the integral of the absolute value of the first term of the line fitted to the data (û_{fit}) of the parameter of the flow of gases, divided by the time range covered by the selected filtered flow rate data points (i.e., a zero-order spline). In some examples, another of the three estimates is an estimate of the device minute ventilation (MV_{device}) represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds. In some configurations, another of the three estimates is an estimate of the device minute ventilation (MV_{device}) represented by the average of the absolute value of the curve fitted to the data of the parameter of the flow of gases (i.e., computed without using splines for data interpolation). In some configurations, the first MV_{device} estimate (i.e., zero-order spline) is most resilient to noise/least dependent upon it, and is most influenced by patient respiratory rate. In some examples, the second MV_{device} estimate (i.e., first-order spline) is very noise-dependent but is less dependent of respiratory rate. In some configurations, the third MV_{device} estimate (i.e., wherein there are no splines/interpolation and the estimate is a direct averaging of a series of instantaneous absolute values) is highly affected by noise and is independent of respiratory rate. It is further noted that all three estimates are flow-variant, meaning they will vary according to the blower flow output.

In some configurations, the method 1400 can skip step 1408 and the pre-processed flow data can directly proceed to step 1410 wherein the method 1400 can conduct a direct computation of the average of the selected pre-processed flow rate data points.

Once the method 1400 obtains the three estimates of the device minute ventilation (MV_{device}) at step 1410, a filter can be applied to the device minute ventilation (MV_{device}) to average the instantaneous device minute ventilation (MV_{device}) at step 1412. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps.

In the method 1400, there are three measurements (i.e., the first MV_{device} estimate, the second MV_{device} estimate, and the third MV_{device} estimate) and three unknown values or signal components that can constitute and/or contribute to the device minute ventilation signal estimated. These unknown values and signal components can include, for example, noise, respiratory rate (e.g., the speed of flow change induced by the patient), and the underlying device minute ventilation signal. Therefore, there exists a set of three analytic expressions or equations that may be solved simultaneously to derive expressions for noise, respiratory rate, and device minute ventilation. In some configurations, simultaneously solving the three analytic expressions or equations can be highly computationally expensive and therefore demanding on processing hardware in embedded device applications, such as in medical devices. In some configurations, in the method 1400, the algorithm of the method 1400 first proceeds to step 1414 to normalize the three MV_{device} estimates according to the number of data points used in each of the estimations. In some examples, the method 1400 can include step 1416 wherein a noise correction factor can be computed, wherein the noise correction factor has a relationship with the signal-noise ratio. The calculation of a noise correction factor in step 1416 can be similar to any of those disclosed in PCT Application Publication WO/2020/178746, filed 4 Mar. 2020, which is incorporated by reference in its entirety. In some configurations, a noise correction factor that is related to one or more of the normalized MV_{device} estimates may be computed.

In some configurations, the method 1400 can include step 1418 wherein the algorithm can use a pre-defined fitted curve that relates the normalized minute ventilation estimates and the noise correction factor to one of the device minute ventilation (MV_{device}) estimates. In some configurations, the fitting function may comprise, at least partially, some numerically-derived terms. In some configurations, this corrected curve can approximate the output of an analytic expression for the corrected device minute ventilation (MV_{device}). This can provide a corrected device minute ventilation (MV_{device}) that has a minimal noise and respiratory rate dependency.

In some configurations, the method 1400 can include 1420 wherein the corrected device minute ventilation (MV_{device}) is normalized using the device flow rate. In some examples, the same data that was previously used in the prior steps (i.e., good-quality, pre-processed flow rate data) is used to reach the corrected device minute ventilation (MV_{device}) and provide an estimate of device minute ventilation that is flow-invariant and independent of the device flow rate. As mentioned previously, the corrected device minute ventilation (MV_{device}) can be independent of nasal cannula fit and provides a versatile indicator of patient minute ventilation.

### 2.7 WOB Indicators—as a Function of or Relative to a Healthy Person's Measures

In some configurations, any of the described WOB indicators or measures above may be converted to a metric or measure that is a function of a notional healthy person's measures or presented or represented as a metric or measure relative to such a healthy person's measures. In such configurations, the controller of the respiratory apparatus or other processing device implementing or executing the WOB indicator algorithms can take extra steps to convert, transform or otherwise represent the WOB indicators or measures as a function of or relative to a healthy person's measures, based on stored (e.g., in memory of the apparatus) or otherwise accessible comparative data relating to a healthy person.

In one configuration, any of the example WOB indicators may be presented or represented as a ratio or percentage of what would be expected from an ‘average healthy person’. For example, the WOB indicator data may be transformed, converted, or presented so as to represent patient breathing performance as a ratio of ideal healthy value(s). In some configurations, this form of representation of the disclosed WOB indicators may have the advantage of familiarity to clinicians, which may make them more intuitive.

In some configurations, one or more of the WOB indicators described above relating to a nominal ‘average healthy person’ may be computed from a nominal person (or range of different sizes of nominal persons) with an average cannula in a controlled environment and pre-programmed or otherwise stored in memory of or accessible to the respiratory apparatus.

In alternative configurations, WOB indicators could be estimated for a specific patient size using a mixture of estimated, detected, and/or manually-input parameters. For example, measurable flow and pressure parameters (e.g., Q_{tube}, P_{blower}) could be used in combination with measures or estimates of patient size and approximate percentage of nostril occlusion that are manually input by a clinician. Alternatively or additionally, in another example, patient size may be estimated by the device based on the flow rate setting and nostril occlusion may be estimated or approximated using knowledge of the cannula size (e.g. which may be manually input) and patient size.

In another example configuration, a table (or other suitable data structure) of patient parameters and corresponding WOB measures, if they were healthy, could also be stored in memory on the respiratory apparatus and accessed when needed in order to allow the generation of WOB indicators or measures as a function of or relative to a healthy person's measures.

In one example configuration, the respiratory apparatus or controller of the respiratory apparatus may be configured to periodically or continuously compute or calculate the ratio or percentage of a patient WOB measure value and a nominal healthy WOB measure value. Any of the applications described in the following section may be applicable to this ratio or percentage, not only the individual WOB measures.

### 2.8 Applications of WOB Indicators

### Overview

Any of the patient WOB indicators, data, or trend data disclosed herein, including the patient WOB indicator expressed as ratios or percentages relative to a healthy person's data, may be generated and used by the respiratory apparatus in one or more various applications or functions, examples of which are discussed further below. In some configurations, these WOB indicators are generated and used by one or more applications or functions during a respiratory therapy session being undertaken by a patient using the respiratory apparatus. In some configurations, applications or functions may utilise and/or process WOB indicator data generated and stored during a respiratory therapy session, for post-therapy processing and/or storage, such as sending or transmitting the WOB indicator data and/or related therapy data to a remote or cloud computing system, e.g., a patient and/or device management platform.

Examples of various applications and/or functions that may utilise and/or process the WOB indicators discussed above and/or generated by the algorithms disclosed above will now be explained in further detail. In some examples explained further below, the one or more WOB indicators or associated data may be used for any one or more of the following actions:
Display of the WOB indicator data and/or WOB trend data on the respiratory apparatus or an associated device (e.g., on a display screen and/or GUI of the apparatus or transmitted for display on an associated device or a device in data communication with the apparatus).
Triggering or generating alarms or notifications or suggestions (visual, audible, and/or tactile) on the respiratory apparatus or an associated device in data communication with the apparatus.
Triggering or generating one or more alerts, alarms, and/or notifications based at least partly on the determined WOB indicator data and/or WOB trend data, and one or more thresholds. The alerts, alarms, and/or notifications may be audible, visual, and/or tactile.
Triggering or generating one or more alerts, alarms, and/or notifications comprising data indicative of suggested adjustments or changes to therapy settings and/or apparatus settings based at least partly on the WOB indicator data and/or WOB trend data, and one or more thresholds.
Generating reports based on the WOB indicator data and/or WOB trend data.

Any one or more of the example applications and/or functions discussed above or below may be used in combination by the respiratory apparatus.

### First Example Application-Display of WOB Data

In this example, the WOB indicator data generated by the respiratory apparatus may be displayed on a display screen or user interface (e.g. Graphical User Interface-GUI) of the respiratory apparatus, or may be transmitted for display on an associated remote device or system in data communication with the apparatus. As discussed above, the raw or absolute patient WOB indicator may be displayed, and/or a patient WOB ratio or percentage indicator relative to healthy WOB data may be displayed. Additionally or alternatively, one or more WOB trends or trend data (e.g. ‘WOB increasing’, ‘WOB decreasing’, ‘WOB stable’) relating to the WOB data may be displayed in isolation or concurrently with the WOB indicator data.

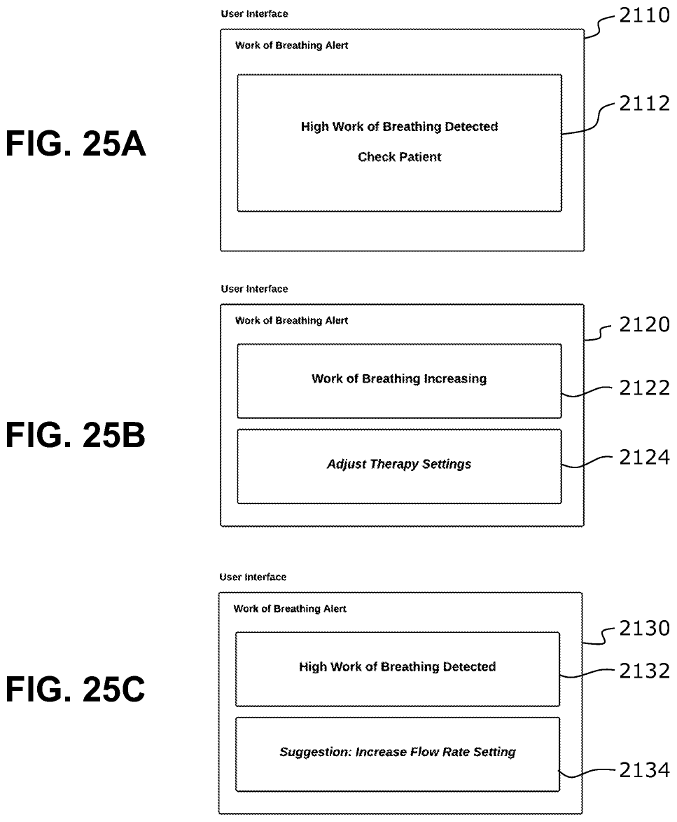

Referring to FIG. 24, an example GUI 2100 is shown which includes a WOB monitor screen. In this example, the WOB monitor screen includes a first GUI element 2102 that is configured to display the WOB indicator data. In this example, the WOB indicator data may be a patient WOB ratio or percentage indicator relative to nominal healthy WOB data, but may alternatively be raw or absolute WOB indicator data. In this example, the WOB monitor screen also includes a concurrently displayed second GUI element 2104 that is configured to display the corresponding WOB trend data relating to the displayed WOB data in the first GUI element 2102. The GUI elements 2102, 2014 may display their respective data in any suitable format or combination of formats including, but not limited to, numerically, graphically, textually, icons, colours, and/or animations, for example.

In a configuration, the respiratory apparatus may be configured to display WOB data (e.g. WOB indicator data and/or WOB trend data) on the display or user interface of the respiratory apparatus at the end of each therapy session or some other configurable or predetermined time.

In a configuration, the respiratory apparatus may be configured to process the WOB data across multiple therapy sessions or time periods for a patient, and may generate comparative data and/or aggregated data and/or statistical data representing or indicative of statistics, changes, and/or trends in the WOB data for the patient over multiple therapy sessions and/or with regard to other desired time periods of captured data (e.g. number of days or weeks). The comparative, aggregated, and/or statistical data may be stored on the respiratory apparatus and/or transmitted to a remote device or server for storage and/or further processing. Additionally or alternatively, the comparative, aggregated, and/or statistical data may be displayed or presented on the display of the respiratory apparatus at the end of a therapy session and/or at some other configurable or predetermined time and/or based on a condition or event.

### Second Example Application-Display of Notifications and/or Suggestions

In this example, the WOB data and/or related notifications and/or suggestions generated or triggered based on the WOB data may be displayed for the user, patient and/or clinicians or clinical staff (e.g. respiratory therapists, nurses etc). The WOB data, notifications, and/or suggestions may be displayed on the display screen or user interface (e.g. GUI) of the respiratory apparatus and/or transmitted for display on a remote device or system in data communication with the apparatus (e.g. a patient and/or device management system, and/or a portable electronic device such as smartphone, tablet, laptop, wearable smart device, or the like).

In one configuration, a GUI of the respiratory apparatus may be configured to display or present one or more live or real-time generated WOB indicators based on any of those disclosed above. This may also prompt a user to see if the therapy settings or changes to the therapy settings (e.g. flow rate setting and/or gas flow oxygen concentration related settings such as FiO₂ and/or FdO₂ settings for example) of the respiratory apparatus favourably change the patient's WOB (e.g. result in a low, lower, or decreasing WOB) in real-time. This configuration may enable a user or clinician to fine-tune therapy settings of the respiratory apparatus for the patient with the objective of reducing WOB.

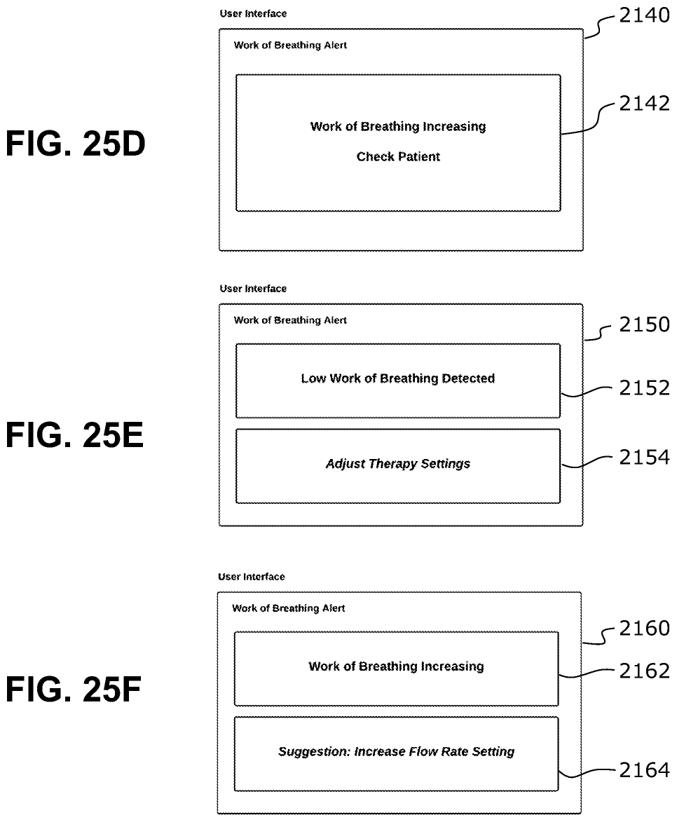

In another configuration, one or more audible and/or visual alerts, warnings, notifications, prompts or similar may be presented or displayed concurrently with WOB indicators or data on the display screen or GUI. Audible alerts, alarms and/or notifications may be provided via an audio output device of the apparatus. For example, if patient WOB is increasing/has increased with a new flow rate setting, an appropriate alert may be presented or delivered. With reference to FIGS. 25A-25F, various GUI examples of such configurations and/or notifications and/or alerts will be described.

FIG. 25A shows a first example GUI 2110 in which the WOB data has triggered a message 2112 indicating that a high work of breathing has been detected, and that the patient should be checked.

FIG. 25B shows a second example GUI 2120 in which the WOB data has triggered a first GUI element 2122 to display a trend notification indicating that the work of breathing of the patient is increasing, and a second GUI element 2124 comprising notification or suggestion data to adjust the therapy settings. The notification data may include data indicating how to adjust one or more of the therapy settings of the respiratory apparatus to reduce the work of breathing, for example.

FIG. 25C shows a third example GUI 2130 in which the WOB data has triggered a first GUI element 2132 to display a message indicating that a high work of breathing has been detected, and a second GUI element 2134 comprising a notification or remedial suggestion to the user or clinician to increase the flow rate setting of the respiratory apparatus. In this example, the GUI displays a WOB alert and corresponding remedial data or suggestion data providing information on how to resolve or remedy the alert, e.g. increasing the flow rate setting may assist in lowering the patient's current work of breathing as represented by the calculated WOB data.

FIG. 25D shows a fourth example GUI 2140 in which the WOB data has triggered a message or trend notification 2142 indicating that a trend of increasing work of breathing has been detected, and that the patient should be checked.

FIG. 25E shows a fifth example GUI 2150 in which the WOB data has triggered a first GUI element 2152 to display a notification or message indicating that a low work of breathing has been detected, and a second GUI element 2154 comprising a notification or remedial suggestion to adjust the therapy settings. The notification data may include data indicating how to adjust one or more of the therapy settings of the respiratory apparatus in response to the low work of breathing detected.

FIG. 25F shows a sixth example GUI 2160 in which the WOB data has triggered a first GUI element 2162 to display a trend notification indicating that the work of breathing of the patient is increasing, and a second GUI element 2164 comprising a notification or remedial suggestion to the user or clinician to increase the flow rate setting of the respiratory apparatus. In this example, the GUI displays a WOB alert and corresponding remedial data or suggestion data providing information on how to resolve or remedy the alert, e.g. increasing the flow rate setting may assist in halting and/or reversing the current trend of the patient's increasing work of breathing.

As discussed above, the notification data or information provided in the WOB notifications, alerts, and/or suggestion display screens may be provided or presented in any suitable for or combination of visual forms including, but not limited to, numerical values, textual information, graphical form or formats, continuous trend lines, plotted or graphed data over time, icons, animation, and/or colour-coded information for example. Additionally or alternatively, the notification data may be provided audibly and/or with audible cues or voice commands, for example.

### Third Example Application-Reporting of WOB Data or Notification Data and Generation Of Reports

In some configurations, the generated WOB data (e.g. WOB indicator data and/or WOB trend data) and/or notification data (e.g. alerts, alarms, notifications triggered based on the comparison of the WOB data to one or more thresholds) may be reported or transmitted by the respiratory apparatus to one or more remote devices or systems (e.g. patient and/or device management system, and/or other personal or portable electronic device such as a smartphone, tablet, laptop, wearable device). In one configuration, the respiratory apparatus may be configured to report or transmit the WOB data and/or notification data instantly in real-time, periodically, on demand or at request, at configurable intervals, or automatically in response to particular events or actions.

In one example configuration, the respiratory apparatus is configured to report or transmit the WOB data and/or notification data at the end of or after each therapy session. In one example, the respiratory apparatus is configured to report or transmit the WOB data and/or notification data while the apparatus is operating in a drying mode after a therapy session or any other suitable time after the therapy session has ended. In one configuration, a drying mode of the respiratory apparatus refers to a mode of the apparatus in which the respiratory apparatus is dried out following a therapy session, for example by the controller running the blower or flow generator but not the humidifier.

In one example configuration, the respiratory apparatus is configured to report or transmit the WOB data and/or notification data from a previous therapy session during the warm-up mode of a subsequent therapy session. For example, during the warm-up mode of a new therapy session, the respiratory apparatus may be configured to report or transmit the WOB data and/or notification data generated from the previous therapy session or a plurality of previous sessions. In one configuration, a warm-up mode of the respiratory apparatus refers to a mode of the apparatus in which a humidifier of the apparatus (if present) is brought up to operating temperature by the controller prior to a therapy session commencing.

Any of the above notification data (e.g. alerts, notifications, suggestions etc) triggered in response to the calculated or determined WOB data may additionally or alternatively be transmitted for display or presentation on a remote device or system that is in data communication, directly or indirectly, with the respiratory apparatus. Additionally or alternatively, the WOB data generated by the respiratory apparatus may trigger such notification data to be presented on a remote device or system, e.g. the remote device or system may trigger the display or presentation of the notification data in response to receiving and processing of the WOB data from the respiratory apparatus. By way of example, the remote device or system may be any suitable electronic device or system having a visual (e.g. display screen), audible and/or tactile user interface, including but are not limited to, a mobile phone, smart phone, tablet, laptop, pager, personal computer, wearable device, or any other suitable electronic device.

In some configurations, the WOB data and/or associated triggered notification data may be transmitted by the respiratory apparatus to a remote device or system for presentation.

In some configurations, the WOB data and/or associated triggered notification data may be transmitted to a remoted cloud or server-based patient and/or device management system, which may process the incoming data and then relay on or push the WOB and/or notification data to one or more other electronic devices or systems (e.g. clinician electronic devices or systems such as a smartphone, tablet, laptop, computer, wearable device, or the like). In some configurations, the patient and/or device management system may be configured to receive the WOB data from the respiratory apparatus, process the WOB data, and cause (e.g. push, trigger or generate) notification data to be presented on one or more remote electronic devices or systems (e.g. clinician electronic devices or systems).

In some configurations, the remote server or device (e.g. patient and/or device management system) receiving WOB data and/or notification data from the respiratory apparatus may be configured to generate one or more patient reports based on the received

WOB data (e.g. WOB indicator data and/or trend data). The generated reports may be any suitable type and/or format of report including, but not limited to, report data, displayed reports, compiled reports, electronic reports, printable reports, numerical reports, graphical reports. The reports may comprise data representing the WOB data and/or notification data for a single therapy session and/or across multiple therapy sessions and/or across a selectable or configurable time period (e.g. number of days or weeks)

In some configurations, the generated reports may comprise comparative data and/or aggregated data and/or statistical data relating to the received WOB data and/or notification data for a patient based on one or more therapy sessions and/or over desired time periods (e.g. number of days or weeks).

### Fourth Example Application-Therapy Parameter Setting Adjustment Suggestion in Response to WOB Indicator

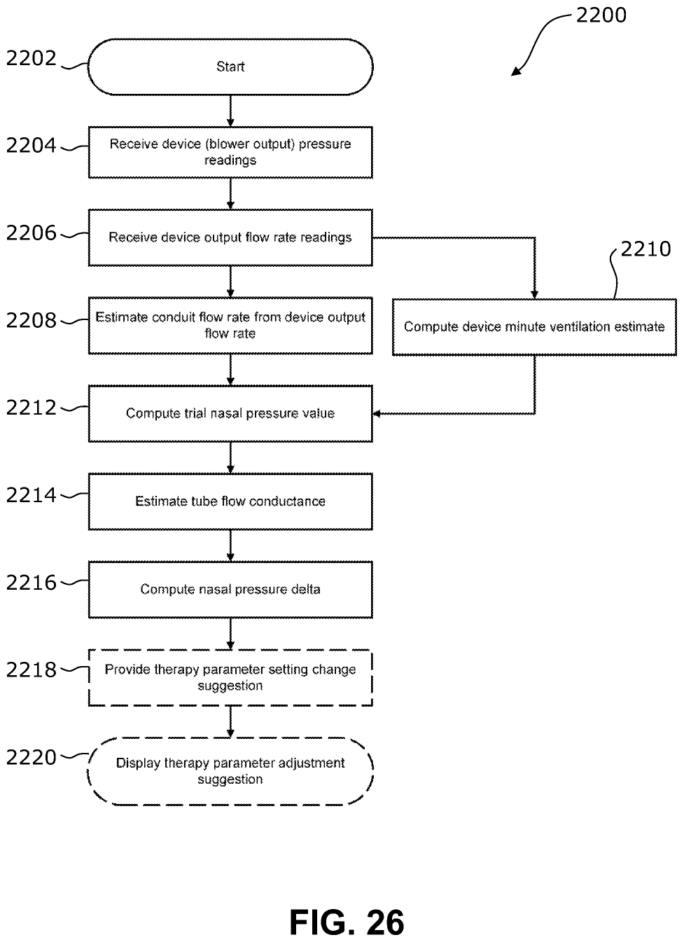

Referring to FIG. 26, an example method 2200 implemented by the respiratory apparatus (e.g. a controller of the respiratory apparatus) for calculating a WOB indicator in accordance with the previous disclosure above will be described, and which results in the respiratory apparatus generating a suggestion for a therapy parameter setting adjustment in response to the calculated WOB indicator. The ordering of the steps described is not essential in all configurations, and some steps may occur in parallel rather than sequentially. The detail and alternatives associated with each of the steps has been described above, and will not be repeated for brevity.

In this example method 2200, the process starts at step 2202, with the controller of the respiratory apparatus receiving pressure data at step 2204, e.g., from one or more pressure sensors of the apparatus. In this configuration, the pressure data is indicative of the pressure at the output of the blower P_{blower}. The controller then receives flow rate data indicative of the apparatus output flow rate as shown at step 2206, e.g., from one or more flow rate sensors in the flow path of the apparatus. The controller is then configured to calculate or estimate a conduit or tube flow rate Q_{tube} based at least partly on the received apparatus output flow rate data as shown at step 2208. In this example, the controller is also configured to generate an estimate of the device minute ventilation MV_{device} based at least partly on the apparatus output flow rate data as shown at step 2210.

In this example, the controller then proceeds to determine or calculate an estimate of a trial nasal pressure value Pₙₒₛₑ,guess as shown at step 2212. An estimate of tube flow conductance C_{tube} is then determined based at least partly on the data or values for Q_{tube}, P_{blower}, and P_{nose,guess}, as shown at step 2214. Following this, the controller is configured to calculate a nasal pressure fluctuation WOB indicator ΔPₙₒₛₑ based at least partly on the values for Q_{tube}, C_{tube}, and MV_{device} as shown at step 2216, and as previously described. Additionally, or alternatively, the controller may undertake further processing steps to generate one or more of the other WOB indicators described above in sections 2.3 and 2.4.

In this example method 2200, the controller is then configured to optionally process the ΔPₙₒₛₑ WOB indicator or other calculated WOB data, and may generate therapy parameter setting change or adjustment suggestions. The generated therapy parameter setting change or adjustment suggestions may be based at least partly on or in response to the generated WOB data or based on further processing of the WOB data relative to one or more thresholds or similar as shown at step 2218. For example, if the WOB data is outside of configurable limits or is demonstrating unfavourable trends, the controller may generate change or adjustment suggestions, as described above.

If therapy parameter setting changes are suggested or triggered at step 2218, the controller may be optionally configured to display or present those suggested therapy parameter setting changes on the display screen of the respiratory apparatus and/or may transmit them for display on one or more remote devices or systems, as discussed above, as shown at step 2220. In one configuration, the respiratory apparatus may be configured with an additional optional step of enabling a user or clinician to acknowledge and confirm or reject the suggested therapy parameter setting changes, depending on whether they want the suggested setting changes to be applied by the controller. For example, a user or clinician may confirm or reject the suggested setting changes via user interaction with a user interface of the respiratory apparatus and/or remote device or system.

In a configuration, the controller may be configured to process the WOB data (e.g. WOB indicator data and/or WOB trend data), and may then generate a notification, alert or alarm suggesting a therapy parameter setting adjustment based on comparing the WOB data to one or more thresholds. The controller may suggest changes to any one or more therapy settings or apparatus settings such as, but not limited to, flow rate settings and gas flow oxygen concentration settings (e.g. FiO₂ and/or FdO₂ settings for example that control the oxygen concentration in the gas flow provided to the patient). The therapy parameter setting adjustment suggestions may include directions such as ‘increase flow rate’ or ‘decrease flow rate’ or ‘increase oxygen concentration’ or ‘decrease oxygen concentration’ for example, depending on the response or remedial change needed in view of the WOB data comparison to the one or more thresholds. As discussed above, the suggested therapy setting adjustments may be presented or displayed on the respiratory apparatus and/or transmitted to a remote device or system for processing and/or display.

### Fifth Example Application-Disconnection Alarm or Notification

In another example, the controller may be configured to process any of the one or more calculated WOB indicators described above to detect disconnection of any part of the flow path (e.g., the patient breathing circuit and/or patient interface), and/or disconnection or detachment of the patient from the patient interface (e.g., nasal cannula).

For example, in one configuration, if the ΔPₙₒₛₑ WOB indicator (and by extension, the ΔPₙₒₛₑ*Q_{breath} WOB indicator or ΔPₙₒₛₑ*σ WOB indicator, etc.) reaches zero or crosses a pre-determined threshold (e.g., near zero) for a sufficient period of time (i.e., no patient breathing is detected), then a disconnection may have occurred. If such a disconnection is detected based on the WOB data, any suitable response may be initiated or triggered. For example, the controller may trigger the presentation of audio and/or visual alarms and/or displaying of a notification (e.g., textual and/or animation) on a display screen to suggest corrective action. Alternatively, or additionally, an alarm (visual, audible, and/or tactile) may be triggered on a remote device or system such as, but not limited to, a mobile phone, tablet, laptop, pager, or other suitable device (further examples of which have been described previously).

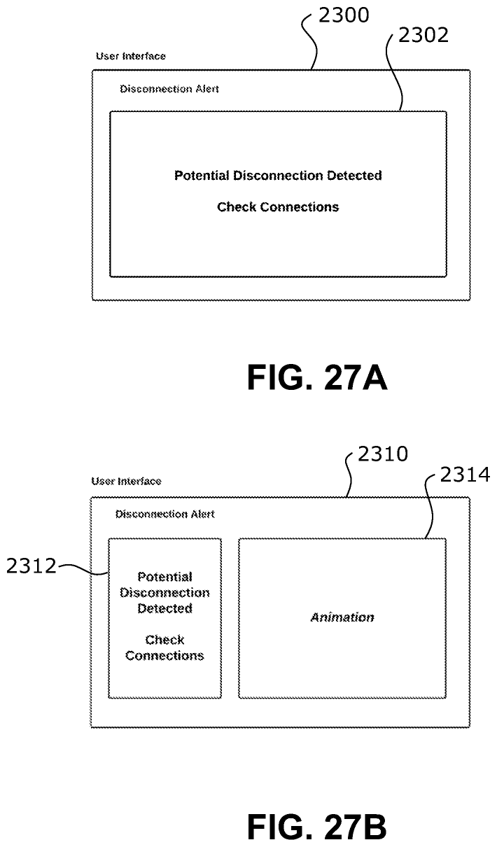

Referring to FIGS. 27A and 27B, some examples of possible GUI screen disconnection alert notifications for display on a display screen of the respiratory apparatus or remote device are shown.

FIG. 27A shows a first example GUI 2300 in which the WOB data has triggered a disconnection alert notification screen comprising information indicating that a potential disconnection event has been detected and prompting the user or clinician to check the patient circuit (e.g., breathing tube and/or patient interface) connections along the air circuit and/or to check for patient detachment from the patient interface.

FIG. 27B shows a second example GUI 2310 in which the WOB data has triggered a disconnection alert notification screen comprising a first GUI element 2312 comprising textual information indicating that a potential disconnection event has been detected and prompting the user or clinician to check the patient circuit like in FIG. 27A example. Additionally, a second GUI element 2314 may be displayed alongside or concurrently with the first GUI element comprising an animation or other imagery, which may prompt the user to check the patient breathing circuit or otherwise alert them visually to a potential disconnection event.

### Sixth Example Application-Configurable Alarm Settings

In another example, the controller of the respiratory apparatus may be configured with one or more configurable alarm or triggering thresholds against which the WOB data generated may be compared and then actions may be taken depending on those comparisons.

By way of example, any of the WOB indicators or data may be compared against one or more thresholds associated with one or more respective notifications, alerts and/or alarm events. For example, the controller may be configured with one or more particular notifications, alerts or alarm events that are triggered if the WOB data meets or satisfies the threshold requirements or threshold rule. The threshold rules or requirements for each notification, alert and/or alarm event may be based on a single threshold, a threshold range, by upper and lower threshold limits, or a threshold function based on one or more parameters and/or conditions (e.g., an alert/alarm/notification is triggered if WOB data exceeds upper limit for a specific time period, or if WOB data exceeds upper limit more than x times within a specific time period). Whether the notification, alert and/or alarm event is triggered may depend on whether the threshold rule or function is satisfied after the WOB data or indicator(s) are compared with the threshold rule or function and the associated threshold limit(s).

In some configurations or for some notification, alert or alarm events, the threshold limit or limits or threshold function parameters may be pre-programmed or pre-configured. In other configurations or for some notification, alert or alarm events, the threshold limit or limits or threshold function parameters may be user or clinician configurable. In such configurations, the threshold limit(s) or threshold function parameters may be configurable and/or adjustable via a user interface (e.g., GUI) presented on the display screen of the respiratory apparatus.

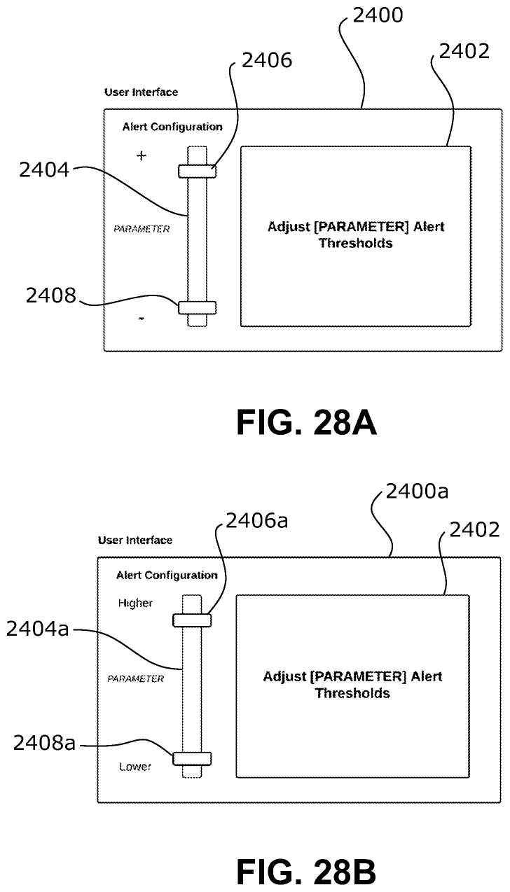

Referring to FIGS. 28A and 28B, some examples of possible GUI screens that are operable to adjust threshold parameters for particular notification, alert and/or alarm events are shown.

FIG. 28A shows a first example GUI 2400 that presents a parameter alert threshold configuration or adjustment screen. In this example GUI 2400, a first GUI element 2402 provides a notification or information about the parameter alert or notification threshold that is being adjusted. For example, the parameter alert or notification or alarm might be selected from examples including, but not limited to, the following: ‘high WOB’, ‘low WOB’, and/or ‘possible disconnection’. In this example GUI 2400, a second GUI element 2404 is also provided and which may be a user interactable or operable GUI element(s) or interface that enables adjustment of the one or more thresholds associated with the parameter alert. In this example, the GUI may be presented on a touch-screen user interface, and the user may interact with the GUI elements to configure the thresholds via touch input or interactivity. The user interactable GUI element for adjusting the one or more configurable thresholds may comprise any suitable form of adjustment including, but not limited to, toggle elements to increase or decrease the threshold limit(s), dials or slider scale elements for adjusting the threshold(s), selectable discrete threshold elements for selecting from a range of discrete threshold levels, numerical or categorical input fields for inputting desired thresholds.

In the example shown in FIG. 28A, the threshold is adjusted via a user interactable GUI 2404 with positive (′+′) and negative (‘-’) GUI elements or buttons that may be interacted with to incrementally increase and decrease respectively the alert parameter threshold. In this example, the upper slider element 2406 may represent an upper limit parameter threshold, and a lower slider element 2408 may represent a lower limit parameter threshold. In one configuration, a user may tap, select, or touch either of the upper or lower slider elements 2406, 2408, and may then move the selected slider element to adjust the selected threshold by interacting with the positive (′+′) and negative (‘-’) GUI elements or buttons. Additionally or alternatively, the user may directly interact with the upper and/or lower slider elements 2406, 2408 by sliding and/or dragging the slider elements along the slider bar/range to adjust the respective upper and/or lower limit parameter thresholds.

The second example GUI 2400a shown in FIG. 28B is the same as that shown and described in FIG. 28A, except that it includes a different user-interactable GUI 2404a that includes qualitative adjustments to the threshold limit(s) via operation of ‘higher’ and ‘lower’ GUI elements to increase and decrease the limit(s) respectively. Like the example described for FIG. 28A, a user may select either of the upper and/or lower slider elements 2406a, 2408a and then interact with the ‘higher’ or ‘lower’ GUI elements to adjust the selected parameter threshold, or the user may interact with either of the slider elements 2406a, 2408a directly by sliding and/or dragging the slider elements along the slider bar/range to adjust the associated parameter threshold.

In other example configurations, it will be appreciated that the GUIs in FIGS. 28A and 28B may alternatively be provided with a single interactable slider bar on the slider scale for adjusting a single upper or lower parameter alert threshold.

In one example configuration, the WOB indicator or data may act as a background parameter that is used to set or as an input to guide setting of one or more general respiratory apparatus alarm thresholds (e.g., respiratory rate alarm, minute ventilation alarm, tidal volume alarm, and/or the like). In other example configurations, the WOB alarms may be configured based on the WOB indicator or data, and the WOB alarms may act as surrogate alarms for other parameters such as, but not limited to, respiratory rate, minute ventilation, tidal volume and/or the like. For example, in some configurations, the WOB indicator may be closely related to some of these other parameters. For example, if a patient's respiratory rate or minute ventilation increases, the WOB indicator should generally also increase correspondingly. Likewise, if a patient's respiratory rate or minute ventilation decreases, the WOB indicator will generally also decrease. This may be advantageous in some configurations because detecting some respiratory parameters can be quite difficult in unsealed respiratory systems (such as nasal high flow systems). The WOB indicators described may, in some cases, be more reliably computed and yet still dependent on the underlying patent respiratory parameter(s), thus making them useful as surrogate or proxy indicators that can be used to trigger one or more respiratory parameter alarms, as further explained later.

In one example configuration, after the respiratory apparatus is set-up and running a respiratory therapy session with a patient, an initial WOB indicator or data may be computed or calculated. The respiratory apparatus may then display or visualise the WOB indicator or data or another relevant parameter (e.g., minute ventilation) on its display, and provide suggestions for upper and lower bounds or threshold limits based at least in part on the WOB indicator or data.

In one example, an alarm may be provided that is triggered based on comparing the ΔPₙₒₛₑ WOB indicator or data to an associated threshold. This ΔPₙₒₛₑ WOB indicator alarm may be considered to act as a surrogate for a patient minute ventilation alarm (actual patient minute ventilation, as compared to the device or nasal minute ventilation measures as described previously) or a respiratory rate alarm. An alarm threshold based on the Pₙₒₛₑ WOB indicator and which acts as a surrogate for a patient minute ventilation or respiratory rate alarm may have advantages, in some configurations. Estimation of the ΔPₙₒₛₑ WOB indicator may potentially be more accurate and/or more informative of concerning patient breathing changes.

In one example, the ΔPₙₒₛₑ WOB indicator in use as an alarm may be analogous to a minute ventilation alarm. In this example, the ΔPₙₒₛₑ WOB indicator is proportional and closely related to the minute ventilation (MV) of the patient (and

$$M V = \frac{{V}_{T}}{R R} ,$$

where V_{T} is tidal volume). As such, the device may be configured such that certain alarm thresholds for the ΔPₙₒₛₑ WOB indicator value function as an alarm system for high or low MV. In this case, a larger ΔPₙₒₛₑ WOB indicator magnitude corresponds to a higher MV, and vice versa.

In another example, because ΔPₙₒₛₑ is proportional and closely related to MV, a ΔPₙₒₛₑ WOB indicator alarm could be configured to act as a respiratory rate (RR) alarm. For example, because

$$Δ {P}_{nose} ∝ M V ∝ \frac{{V}_{T}}{R R} ,$$

it may be said that

$$R R ∝ \frac{1}{Δ {P}_{nose}} ,$$

if tidal volume is assumed to be relatively constant during the provision of respiratory therapy. Thus, decreasing ΔPₙₒₛₑ (magnitude) might be indicative of increasing RR and vice-versa. As the patient takes shorter, more frequent breaths, their RR will increase while MV (proportional to ΔPₙₒₛₑ) will decrease.

In other examples, any one or more of the previously discussed WOB indicator examples (e.g., ΔPₙₒₛₑ, ΔPₙₒₛₑ*Q_{breath}, and/or ΔPₙₒₛₑ*σ) may be monitored against thresholds for use as surrogate alarms for patient minute ventilation or respiratory rate. For example, any one or more of the WOB indicator examples may be compared against one or more thresholds that are configured or calibrated for patient minute ventilation alarms and/or respiratory rate alarms. In some such configurations, the WOB indicators may be compared against thresholds without any temporal aspect (e.g., the alarm will trigger if the WOB indicator crosses the threshold at any point). In other such configurations, the WOB indicators may be compared against thresholds with one or more associated additional triggering conditions, such as temporal or trend or other conditions. For example, the alarm thresholds may be configured such that they only trigger if the WOB indicator is above or below a threshold for a predetermined or configurable time period, or if the WOB indicator crosses the threshold a certain number of times within a predetermined or configurable period, or other such conditions.

In some configurations, in regard to WOB indicator alarms, an upper bound or threshold may (at least partially) correspond to an undesirably high WOB that requires a change in therapy parameters. A lower bound or threshold may similarly correspond (at least partially) to an undesirably low WOB but may also be used as a disconnection alarm trigger as previously described. As such, the one or more WOB indicators or data may be compared against one or more thresholds, and may trigger different alarms, alerts or notifications depending on whether the WOB indicator is above or below the thresholds (depending on the threshold function or criteria).

In one example configuration, a clinician may choose to accept notification, alert and/or alarm thresholds that are default or which the respiratory apparatus suggests, disregard them entirely, use them in tandem with their own preferred thresholds or other alert triggers (e.g., SpO₂ alarm), or make adjustments to the default or suggested thresholds before accepting them. In this example, the clinician may perform these alarm threshold setting adjustments or threshold configurations or confirmations via the user interface (e.g., GUI) of the respiratory apparatus, or remotely via the user interface of another electronic device or system that is in data communication with the respiratory apparatus.

In some configurations, the WOB indicator alarms, alerts or notifications may be configured with a plurality of or multiple upper and lower bounds or thresholds. For example, the alarms, alerts or notifications may be configured with cascading or nested thresholds or threshold ranges, or inner and outer threshold ranges, or a plurality or series of progressive or escalating thresholds in which the nature of the triggered alarm, alert or notification associated with each respective threshold is a function of or dependent on the nature, position, priority or extremity of the threshold on the overall threshold scale. In one example configuration, a first upper bound or threshold may simply trigger a notification or suggestion to adjust therapy parameters, while a second, higher upper bound or threshold may trigger a high-priority alert, as the value of this higher upper bound or threshold may correspond to a WOB indicator magnitude that is indicative of a severe hyperventilation or other serious medical event, for example.

In one example configuration, the default or suggested upper/lower bounds or threshold limits of the respiratory apparatus may be recalibrated or dynamically changed for each therapy session, after a time period elapses, or after a number of therapy sessions have passed. In other words, the threshold suggestions may change daily, weekly, monthly, or over any other suitable time period (which may also be configurable). In one example, the recalibration of the suggested thresholds may be based at least in part on changes in one or more computed WOB indicators or data, and/or WOB trend data. By way of example, the controller may be configured such that a continued downward trend in one or more of the WOB indicators or data over one or more therapy sessions may cause the suggested upper/lower bounds or thresholds to be narrowed (i.e., a narrower acceptable range can be tolerated), shifted down, shifted up, or otherwise changed according to any combination thereof.

In some example scenarios, default values for upper/lower bounds or thresholds may be set or configured across a fleet of respiratory apparatuses according to a hospital, health system, or clinician protocol. In one configuration, the default thresholds may be pre-programmed during manufacture or remotely configured, e.g. via a cloud or server-based patient and/or device management system or platform.

In one configuration, the default values of the upper/lower bounds or thresholds may be stored in the respiratory apparatus memory or memory of a remote device or system that is used to configure the respiratory apparatus.

In some example configurations, the alerts, notifications and/or alarms may be configured to selectively present on different devices or systems based on the threshold crossed. For example, as mentioned, a first upper bound or threshold may trigger a notification to adjust therapy parameters and may only be displayed on the display of the respiratory apparatus. A second upper bound or threshold, corresponding to a more severe threshold, may triggered the alarm or notification to be presented on more than one device or system such as, for example, on one or more remote devices, in addition to being displayed on the respiratory apparatus.

In the above, although upper and lower bounds or threshold have been discussed together, they may be configured individually or selectively in some configurations. For example, a clinician or user may selectively configure the lower bound(s) or thresholds for disconnection alarming but may not adjust the higher bound(s) or thresholds associated with other alarms or notifications (e.g. high WOB).

### 3. Terminology and Definitions

The phrases ‘computer-readable medium’ or ‘machine-readable medium’ as used in this specification and claims should be taken to include, unless the context suggests otherwise, a single medium or multiple media. Examples of multiple media include a centralised or distributed database and/or associated caches. These multiple media store the one or more sets of computer executable instructions. The phrases ‘computer-readable medium’ or ‘machine-readable medium’ should also be taken to include any medium that is capable of storing, encoding or carrying a set of instructions for execution by a processor of a computing device and that cause the processor to perform any one or more of the methods described herein. The computer-readable medium is also capable of storing, encoding or carrying data structures used by or associated with these sets of instructions. The phrases ‘computer-readable medium’ and ‘machine readable medium’ include, but are not limited to, portable to fixed storage devices, solid-state memories, optical media or optical storage devices, magnetic media, and/or various other mediums capable of storing, containing or carrying instruction(s) and/or data. The ‘computer-readable medium’ or ‘machine-readable medium’ may be non-transitory.

The term ‘comprising’ as used in this specification and claims means ‘consisting at least in part of’ or ‘including, but not limited to’ such that it is to be construed in an inclusive sense as opposed to an exclusive or exhaustive sense. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term ‘and/or’ means ‘and’ or ‘or’, or both.

The use of ‘(s)’ following a noun means the plural and/or singular forms of the noun.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

In the above description, specific details are given to provide a thorough understanding of the embodiments. However, it will be understood by one of ordinary skill in the art that the embodiments may be practiced without these specific details. For example, software modules, functions, circuits, etc., may be shown in block diagrams in order not to obscure the embodiments in unnecessary detail. In other instances, well-known modules, structures and techniques may not be shown in detail in order not to obscure the embodiments.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described above may be operable on any type of general purpose computer system or computing device, including, but not limited to, a desktop, laptop, notebook, tablet, smart television, gaming console, or mobile device. The term “mobile device” includes, but is not limited to, a wireless device, a mobile phone, a smart phone, a mobile communication device, a user communication device, personal digital assistant, mobile hand-held computer, a laptop computer, wearable electronic devices such as smart watches and head-mounted devices, an electronic book reader and reading devices capable of reading electronic contents and/or other types of mobile devices typically carried by individuals and/or having some form of communication capabilities (e.g., wireless, infrared, short-range radio, cellular etc.).

Aspects of the systems and methods described above may be operable or implemented on any type of specific-purpose or special computer, or any machine or computer or server or electronic device with a microprocessor, processor, microcontroller, programmable controller, or the like, or a cloud-based platform or other network of processors and/or servers, whether local or remote, or any combination of such devices.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the above description, a storage medium may represent one or more devices for storing data, including read-only memory (ROM), random access memory (RAM), magnetic disk storage mediums, optical storage mediums, flash memory devices and/or other machine or computer readable mediums for storing information.

The various illustrative logical blocks, modules, circuits, elements, and/or components described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, circuit, and/or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

One or more of the components and functions illustrated the figures may be rearranged and/or combined into a single component or embodied in several components without departing from the scope of the disclosure. Additional elements or components may also be added without departing from the scope of the disclosure. Additionally, the features described herein may be implemented in software, hardware, as a business method, and/or combination thereof.

In its various aspects, embodiments of the disclosure can be embodied in a computer-implemented process, a machine (such as an electronic device, or a general purpose computer or other device that provides a platform on which computer programs can be executed), processes performed by these machines, or an article of manufacture. Such articles can include a computer program product or digital information product in which a computer readable storage medium containing computer program instructions or computer readable data stored thereon, and processes and machines that create and use these articles of manufacture.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this disclosure may comprise, additional to its essential features described herein, one or more features as described herein from each other embodiment of the invention disclosed herein.

This disclosure may also be said broadly to consist in the parts, elements and features referred to or indicated in this disclosure, individually or collectively, and any or all combinations of any two or more said parts, elements or features, and where specific integers are mentioned herein which have known equivalents in the art to which this disclosure relates, such known equivalents are deemed to be incorporated herein as if individually set forth.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A respiratory apparatus configured to provide a flow of gases to a user for respiratory therapy, comprising:
a flow generator configured to generate the flow of gases for the user;
a sensor or sensors that are configured to generate flow parameter data indicative or representative of the flow of gases; and
a controller, wherein the controller is configured to:
receive the flow parameter data;
determine a nasal pressure variation value indicative of the user's average nasal pressure based at least partly on the received flow parameter data;
determine a work of breathing (WOB) indicator based at least partly on the determined nasal pressure variation value; and
initiate one or more actions based at least partly on the determined WOB indicator.

2. A respiratory apparatus according to claim 1 wherein the flow parameter data comprises flow rate data indicative or representative of the flow rate of the flow of gases provided by the flow generator.

3. A respiratory apparatus according to claim 2 wherein the apparatus comprises a flow rate sensor or sensors that are configured to sense and generate the flow rate data.

4. A respiratory apparatus according to claim 3 wherein the flow rate sensor or sensors are positioned or located in or within a flow path of the flow of gases.

5. A respiratory apparatus according to claim 3 or claim 4 wherein the flow rate sensor or sensors are positioned or located at or near an outlet of a blower of the flow generator.

6. A respiratory apparatus according to any one of claims 3-5 wherein the flow rate sensor or sensors are in electrical communication with the controller.

7. A respiratory apparatus according to any one of claims 3-6 wherein the controller is further configured to process the flow rate data to remove noise and/or signal components associated with the flow generator.

8. A respiratory apparatus according to claim 7 wherein the controller is configured to remove noise relating to the effect of a motor on the flow rate data.

9. A respiratory apparatus according to claim 7 or claim 8 wherein the controller is configured to receive data regarding a motor speed, and the flow rate data of the flow of gases is discarded if the motor speed is below a pre-set threshold.

10. A respiratory apparatus according to any one of claims 7-9 wherein the controller is configured to discard the flow rate data if the controller determines the flow rate data parameter of the flow of gases is of insufficient quality.

11. A respiratory apparatus according to claim 10 wherein the flow rate data is determined to be of insufficient quality if it includes large transient peaks.

12. A respiratory apparatus according to any one of claims 1-11 wherein the flow parameter data comprises pressure data indicative or representative of the pressure of the flow of gases at an outlet of the blower of the flow generator.

13. A respiratory apparatus according to claim 12 wherein the apparatus further comprises a pressure sensor or sensors that are configured to sense and generate the pressure data.

14. A respiratory apparatus according to claim 13 wherein the pressure sensor or sensors are positioned or located in or within a flow path of the flow of gases.

15. A respiratory apparatus according to claim 13 or claim 14 wherein the pressure sensor or sensors are positioned or located at or near an outlet of a blower of the flow generator.

16. A respiratory apparatus according to any one of claims 13-15 wherein the pressure sensor or sensors are in electrical communication with the controller.

17. A respiratory apparatus according to any one of claims 12-16 wherein the controller is further configured to determine an initial nasal pressure estimate value indicative or representative of an estimate of the user's nasal pressure based at least partly on the pressure data.

18. A respiratory apparatus according to any one of claims 1-17 wherein the controller is further configured to determine a flow path conductance estimate indicative or representative of an estimate of the conductance of the flow path for the flow of gases between the flow generator and a patient interface.

19. A respiratory apparatus according to claim 18 wherein the controller is configured to determine the flow path conductance estimate at least partly based on an initial nasal pressure estimate value indicative or representative of an estimate of the user's nasal pressure.

20. A respiratory apparatus according to claim 18 or claim 19 wherein the controller is configured to determine the flow path conductance estimate at least partly based on flow rate data indicative or representative of the flow rate of the flow of gases provided by the flow generator.

21. A respiratory apparatus according to any one of claims 18-20 wherein the controller is configured to determine the flow path conductance estimate at least partly based on pressure data indicative or representative of the pressure of the flow of gases at an outlet of the blower of the flow generator.

22. A respiratory apparatus according to claim 18 wherein the controller is configured to determine the flow path conductance estimate at least partly based on flow rate data indicative of representative of the flow rate of the flow of gases provided by the flow generator and motor speed representing the motor speed of a blower of the flow generator.

23. A respiratory apparatus according to claim 18 wherein the controller is configured to determine the flow path conductance estimate at least partly based on flow rate data indicative or representative of the flow rate of the flow of gases provided by the flow generator and pressure data indicative or representative of the pressure of the flow of gases at an outlet of the blower of the flow generator.

24. A respiratory apparatus according to any one of claims 18-23 wherein the controller is configured to determine the nasal pressure variation value at least partly based on the flow path conductance estimate.

25. A respiratory apparatus according to any one of claims 18-24 wherein the controller is configured to determine the nasal pressure variation value at least partly based on flow rate data indicative or representative of the flow rate of the flow of gases provided by the flow generator.

26. A respiratory apparatus according to any one of claims 18-25 wherein the controller is configured to determine the nasal pressure variation value at least partly based on minute ventilation data indicative or representative of the average volume of gases being provided by the flow generator per minute.

27. A respiratory apparatus according to claim 26 wherein the controller is configured to determine minute ventilation data by fitting a plurality of splines to the flow parameter data of the flow of gases, wherein the plurality of splines are fit using the least squares criterion and the minute ventilation data is determined by integrating along the plurality of splines.

28. A respiratory apparatus according to claim 26 wherein the controller is configured to determine minute ventilation data by determining the integral of the absolute value of the first term of a line fitted to the flow parameter data of the flow of gases.

29. A respiratory apparatus according to claim 26 wherein the controller is configured to determine device minute ventilation data by determining the integral of the absolute value of a line fitted to the data of the flow parameter data of the flow of gases, divided by a time range.

30. A respiratory apparatus according to claim 26 wherein the controller is configured to determine device minute ventilation data by determining an average of absolute values of a line fitted to the flow parameter data of the flow of gases across a range of time-points within a time range.

31. A respiratory apparatus according to any one of claims 1-30 wherein the nasal pressure variation value is determined at a frequency selected in the range of 1 Hz to 20 Hz.

32. A respiratory apparatus according to any one of claims 1-30 wherein the nasal pressure variation value is determined continuously as a rolling average value.

33. A respiratory apparatus according to any one of claims 1-32 wherein the apparatus further comprises a non-transitory computer-readable medium that is accessible or in data communication with the controller, and preferably wherein the non-transitory computer-readable medium comprises a non-volatile memory, and preferably wherein the apparatus further comprises a patient nostril model that is stored in the non-volatile memory.

34. A respiratory apparatus according to claim 33 wherein the controller is further configured to determine a user breath flow rate estimate value indicative or representative of the user's breath flow rate at least partly based on flow rate data indicative or representative of the flow rate of the flow of gases provided by the flow generator and the patient nostril model.

35. A respiratory apparatus according to claim 34 wherein the controller is configured to determine the user breath flow rate estimate value at least partly based on a flow path conductance estimate indicative or representative of an estimate of the conductance of the flow path for the flow of gases between the flow generator and a patient interface.

36. A respiratory apparatus according to claim 34 or claim 35 wherein the controller is configured to determine the user breath flow rate estimate value at least partly based on minute ventilation data indicative or representative of the average volume of gases being provided by the flow generator per minute.

37. A respiratory apparatus according to any one of claims 34-36 wherein the controller is configured to determine a nares conductance estimate indicative or representative of an estimate of the conductance of the flow path for the flow of gases between a patient interface and the user's nostrils at least partly based on data indicative of the patient interface size and an estimate of nostril occlusion by the patient interface.

38. A respiratory apparatus according to claim 37 wherein the controller is configured to determine the user breath flow rate estimate value at least partly based on the determined or calculated nares conductance estimate.

39. A respiratory apparatus according to any one of claims 34-38 wherein the controller is configured to determine the work of breathing indicator at least partly based on the nasal pressure variation value and the user breath flow rate estimate signal.

40. A respiratory apparatus according to any one of claims 1-39 wherein the controller is configured to determine a smoothness value indicative or representative of the smoothness of minute ventilation data indicative or representative of the average volume of gases being provided by the flow generator per minute.

41. A respiratory apparatus according to claim 40 wherein the controller is configured to determine the work of breathing indicator at least partly based on the nasal pressure variation value and the smoothness value.

42. A respiratory apparatus according to any one of claims 1-41 wherein the apparatus further comprises a display screen, and preferably wherein the display screen displays a graphical user interface, and/or preferably wherein the display screen is in electrical communication with the controller.

43. A respiratory apparatus according to claim 42 wherein the display screen is removable from the apparatus or a housing of the apparatus.

44. A respiratory apparatus according to claim 42 or claim 43 wherein the controller is configured to display a graphical indicator representing the determined work of breathing indicator on the display screen.

45. A respiratory apparatus according to claim 44 wherein the graphical indicator comprises any one or more of the following: numerical value, text, waveform, illustration, or animation.

46. A respiratory apparatus according to claim 44 or claim 45 wherein the graphical indicator is indicative or representative of whether the determined work of breathing indicator is increasing or decreasing.

47. A respiratory apparatus according to any one of claims 1-46 wherein the controller is configured to trigger or generate an alert, alarm, and/or notification based at least partly on the determined work of breathing indicator and one or more thresholds.

48. A respiratory apparatus according to claim 47 wherein the alert, alarm, and/or notification is triggered or generated based at least partly on determining that the work of breathing indicator has increased above a threshold.

49. A respiratory apparatus according to claim 47 or claim 48 wherein the alert, alarm, and/or notification is triggered or generated based at least partly on determining that the work of breathing indicator has decreased below a threshold.

50. A respiratory apparatus according to claim 49 wherein the threshold is a disconnection detection threshold.

51. A respiratory apparatus according to claim 50 wherein the alert, alarm, and/or notification is triggered or generated based at least partly on determining that the work of breathing indicator has decreased below the threshold continuously for an associated predetermined duration condition.

52. A respiratory apparatus according to any one of claims 47-51 wherein the controller is configured to generate the alert, alarm, and/or notification in a form selected from any one or more of the following: audible, visual, and/or tactile.

53. A respiratory apparatus according to claim 52 wherein the apparatus further comprises an audio output device in electrical communication with the controller, and wherein the controller is configured to generate the alert, alarm, and/or notification audibly via the audio output device.

54. A respiratory apparatus according to claim 52 or claim 53 wherein the controller is configured to generate the alert, alarm, and/or notification visually via a display screen of the apparatus.

55. A respiratory apparatus according to any one of claims 52-54 wherein the controller is configured to send or transmit data representing the alert, alarm, and/or notification to a remote device or system that is in data communication with the apparatus.

56. A respiratory apparatus according to any one of claims 47-55 wherein the controller is operable to configure or adjust any parameters of or associated with the one or more of the thresholds based at least partly on user input via a graphical user interface of a display screen of the apparatus.

57. A respiratory apparatus according to any one of claims 47-56 wherein the controller is configured to generate or provide suggested thresholds and/or parameters associated with the one or more thresholds based at least partly on the work of breathing indicator.

58. A respiratory apparatus according to any one of claims 1-57 wherein the controller is further configured to determine a ratio or percentage representing the work of breathing indicator for the user relative to a nominal equivalent work of breathing indicator of a nominal average healthy person.

59. A respiratory apparatus according to claim 58 wherein the nominal equivalent work of breathing indicator is determined based at least partly on an amplitude of nominal variations in nasal pressure of the nominal average healthy person.

60. A respiratory apparatus according to claim 59 wherein the amplitude of nominal variations in nasal pressure of the nominal average healthy person is determined based at least partly on predetermined physiological parameters of the nominal average healthy person.

61. A respiratory apparatus according to claim 59 or claim 60 wherein the amplitude of nominal variations in nasal pressure of the nominal average healthy person is determined based at least partly on manually-input physiological parameters related to the user.

62. A respiratory apparatus according to any one of claims 59-61 wherein the amplitude of nominal variations in nasal pressure of the nominal average healthy person is determined based at least partly on a nominal measure of nostril occlusion by nominal nasal cannula prongs of a patient interface.

63. A respiratory apparatus according to any one of claims 59-62 wherein the amplitude of nominal variations in nasal pressure of the nominal average healthy person is determined based at least partly on a manually-input measure of nostril occlusion by nasal cannula prongs of a patient interface.

64. A respiratory apparatus according to any one of claims 58-63 wherein the controller is configured to generate one or more alerts, alarms, and/or notifications based at least partly on the value of the ratio or percentage, representing the work of breathing indicator for the user relative to a nominal equivalent work of breathing indicator of a nominal average healthy person, or associated trend data of the ratio or percentage, and one or more thresholds.

65. A respiratory apparatus according to any one of claims 58-64 wherein the controller is configured to display the value of the ratio or percentage and/or trend data relating to the ratio or percentage visually on a display screen of the apparatus.

66. A respiratory apparatus according to any one of claims 58-65 wherein the controller is configured to generate an alert, alarm, and/or notification comprising data indicative of suggested adjustments to one or more therapy settings and/or apparatus settings based at least partly on the value of the ratio or percentage, representing the work of breathing indicator for the user relative to a nominal equivalent work of breathing indicator of a nominal average healthy person, or associated trend data of the ratio or percentage, and one or more thresholds.

67. A respiratory apparatus according to claim 66 wherein the therapy settings and/or apparatus settings comprise a flow rate setting and/or a gas flow oxygen concentration setting.

68. A respiratory apparatus according to any one of claims 1-67 wherein the apparatus further comprises a housing, and wherein the housing comprises or integrates:
the flow generator;
a humidifier that is configured to heat and humidify the flow of gases;
a sensing block or sensor module comprising the sensor or sensors that are configured to generate flow parameter data indicative or representative of the flow of gases; and
the controller.

69. A respiratory apparatus according to claim 68 wherein the sensing block or sensor module comprises a flow rate sensor and a pressure sensor.

70. A method of controlling a respiratory apparatus configured to provide a flow of gases to a user for respiratory therapy, the apparatus comprising:
a flow generator configured to generate the flow of gases for the user;
a sensor or sensors that are configured to generate flow parameter data indicative or representative of the flow of gases; and
a controller, wherein the method is executed or implemented by the controller and comprises the steps of:
receiving the flow parameter data;
determining a nasal pressure variation value indicative of the user's average nasal pressure based at least partly on the received flow parameter data;
determining a work of breathing (WOB) indicator based at least partly on the determined nasal pressure variation value; and
initiating one or more actions based at least partly on the determined WOB indicator.

71. A respiratory therapy system configured to provide a flow of gases to a user for respiratory therapy, comprising:
a flow generator configured to generate the flow of gases for the user;
a sensor or sensors that are configured to generate flow parameter data indicative or representative of the flow of gases;
a breathing conduit operatively coupled to the flow generator and configured to convey the flow of gases from the flow generator to the user;
a patient interface operatively coupled to the breathing conduit; and
a controller, wherein the controller is configured to:
receive the flow parameter data;
determine a nasal pressure variation value indicative of the user's average nasal pressure based at least partly on the received flow parameter data;
determine a work of breathing (WOB) indicator based at least partly on the determined nasal pressure variation value; and
initiate one or more actions based at least partly on the determined WOB indicator.

72. A respiratory apparatus comprising:
a flow generator configured to generate a flow of gases for a user;
a sensor or sensors that are configured to generate flow parameter data indicative or representative of a characteristic or parameter of the flow of gases; and
a controller that is configured to:
control the flow generator to deliver the flow of gases for nasal high flow therapy;
determine a work of breathing (WOB) indicator based at least partly on the flow parameter data; and
initiate one or more actions based at least partly on the determined WOB indicator.

73. A respiratory apparatus according to claim 72 wherein the flow parameter data comprises pressure data indicative or representative of the pressure of the flow of gases.

74. A respiratory apparatus according to claim 73 wherein the pressure data is sensed and generated by one or more pressure sensors that are in data communication with the controller.

75. A respiratory apparatus according to claim 74 wherein the one or more pressure sensors are configured to sense and generate pressure data indicative or representative of the pressure of the flow of gases at an outlet of a blower of the flow generator.

76. A respiratory apparatus according to any one of claims 72-75 wherein the flow parameter data comprises flow rate data indicative or representative of the flow rate of the flow of gases.

77. A respiratory apparatus according to claim 76 wherein the flow rate data is sensed and generated by one or more flow rate sensors that are in data communication with the controller.

78. A respiratory apparatus according to claim 77 wherein the one or more flow rate sensors are position or located at or near an outlet of a blower of the flow generator.

79. A respiratory apparatus according to any one of claims 72-78 wherein the WOB indicator is determined at least partly based on flow parameter data that comprises sensed pressure and/or flow rate data relating to the flow of gases.
